(19) United States
(12) Patent Application Publication
(10) Pub. No.: US 2025/0241592 A1
(43) Pub. Date: Jul. 31, 2025

(54) SYSTEMS, DEVICES, AND METHODS FOR ANALYTE SENSOR INSERTION

(71) Applicant: ABBOTT DIABETES CARE INC., Alameda, CA (US)

(72) Inventors: Vivek S. Rao, Alameda, CA (US); Anthony Lin Chern, Alameda, CA (US); Phillip W. Carter, Oakland, CA (US); Joshua Lindsay, Woodside, CA (US); Tuan Nguyen, San Jose, CA (US); Vincent M. DiPalma, Oakland, CA (US)

(21) Appl. No.: 19/069,585

(22) Filed: Mar. 4, 2025

Related U.S. Application Data
(63) Continuation of application No. 17/460,043, filed on Aug. 27, 2021, now Pat. No. 12,239,463.
(60) Provisional application No. 63/072,743, filed on Aug. 31, 2020.

(51) Int. Cl. A61B 5/00; A61B 5/145

(52) U.S. Cl. CPC A61B 5/6847; A61B 5/14503; A61B 2560/0443; A61B 2560/063

(57) ABSTRACT

An assembly and method for delivery of an analyte sensor including a reusable applicator having a proximal portion and a distal portion are disclosed. The reusable applicator can include a housing, a sensor carrier configured to releasably receive the first analyte sensor, a sharp carrier configured to releasably receive a sharp module, and an actuator movable relative to the housing. The actuator can include three positions: a first position with the sensor carrier and the sharp carrier are at the proximal portion of the reusable applicator, a second position with the sensor carrier and the sharp carrier are at the distal portion of the reusable applicator for delivery of the first analyte sensor, and a third position with the sensor carrier at the distal portion of the reusable applicator and the sharp carrier at the proximal portion of the reusable applicator after delivery of the first analyte sensor from the reusable applicator, wherein the first position, the second position, and the third position are different, and wherein the actuator is configured to be returned from the third position to the first position for delivery of another analyte sensor.

### CROSS-REFERENCE TO RELATED APPLICATIONS

The present application is a continuation of U.S. patent application Ser. No. 17/460,043, filed Aug. 27, 2021, which claims priority to and the benefit of U.S. Provisional Patent Application No. 63/072,743, filed Aug. 31, 2020, both of which are incorporated by reference herein in their entireties for all purposes.

### FIELD

The subject matter described herein relates generally to systems, devices, and methods for using an applicator to insert at least a portion of an analyte sensor in a subject.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While current sensors can be convenient for users, they are also susceptible to malfunctions. These malfunctions can be caused by user error, lack of proper training, poor user coordination, overly complicated procedures, physiological responses to the inserted sensor, and other issues. Some prior art systems, for example, may rely too much on the precision assembly and deployment of a sensor control device and an applicator by the individual user. Other prior art systems may utilize sharp insertion and retraction mechanisms that are susceptible to trauma to the surrounding tissue at the sensor insertion site, which can lead to inaccurate analyte level measurements. These challenges and others described herein can lead to improper insertion and/or suboptimal analyte measurements by the sensor, and consequently, a failure to properly monitor the patient's analyte level.

Moreover, applicators used to insert at least a portion of an in vivo analyte sensors can include several components that are often constructed of a mixture of plastic materials, which can be difficult to separate after use making recycling difficult. Additionally, packaging materials for such applicators must fulfill a number of engineering design requirements, including, providing stringent sealing for shelf life storage requirements that demand tight tolerance components with exotic plastic materials for low moisture vapor transition rate, providing adequate lubricity so that insertion force can be maintained, etc. Furthermore, applicators are often packaged inside a carton with alcohol wipes. As a result, applicators are often manufactured for single use and using non-biodegradable materials making them difficult to recycle and/or not durable enough for reuse.

Thus, a need exists for more reliable sensor insertion devices, systems and methods, that are easy to use by the patient, less prone to error, and reusable. Furthermore, a need exists for an applicator that meets engineering design requirements yet is durable enough to be used multiple times and/or can be recycled.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to an assembly for delivery an analyte sensor including a reusable applicator having a proximal portion and a distal portion. The reusable applicator is configured to deliver a first analyte sensor and includes a housing, a sensor carrier configured to releasably receive the first analyte sensor, a sharp carrier configured to releasably receive a sharp module, and an actuator movable relative to the housing. The actuator includes a first position with the sensor carrier and the sharp carrier at the proximal portion of the reusable applicator, a second position with the sensor carrier and the sharp carrier at the distal portion of the reusable applicator for delivery of the first analyte sensor from the reusable applicator, and a third position with the sensor carrier at the distal portion of the reusable applicator and the sharp carrier at the proximal portion of the reusable applicator after delivery of the first analyte sensor. The first position, the second position, and the third position are different. The actuator is configured to be returned from the third position to the first position for delivery of another analyte sensor.

The reusable applicator can include a drive spring to move the sensor carrier and the sharp carrier from the proximal portion to the distal portion and a retraction spring to move the actuator to the third position. The drive spring can be actuated by movement of the actuator from the first position to the second position. The retraction spring can be actuated by movement of the sensor carrier form the proximal position to the distal position of the reusable applicator.

According to certain embodiments, the reusable applicator can further include a latch to hold the sensor carrier in the proximal portion of the reusable applicator when the actuator is moved from the second position towards the third position. According to certain embodiments, with the actuator in the third position, the sharp carrier is accessible from the proximal portion of the reusable applicator to release the sharp module. The reusable applicator can include a visual indicator of a position of the actuator. The actuator can include a button configured to extend a first predetermined length relative the housing in the first position, a second predetermined length relative the housing in the second position, and a third predetermined length relative the housing in the third position, wherein the third predetermined length is greater than the first predetermined length and the first predetermined length is greater than the second predetermined length. The button can be configured to be opened for removal of the sharp module.

According to embodiments of the present disclosure, the assembly can be made of recyclable material. The reusable applicator can comprise acetal. The assembly can include a sealable container to package the reusable applicator. The sealable container can have a low moisture vapor transition rate. The sealable container can be configured to eliminate the need for a desiccant. The assembly can include an applicator cap sealingly coupled to the housing using a gasketless seal.

According to embodiments of the present disclosure, a method of using an assembly for delivery of an analyte sensor can include providing a reusable applicator having a proximal portion and a distal portion and including a housing, a sensor carrier having a first analyte sensor control device releasably received therein, and a sharp carrier having a sharp module releasably received therein, and an actuator moveable relative to the housing, moving the actuator of the assembly from a first position toward a second position to move the sensor carrier and the sharp carrier from the proximal portion of the reusable applicator to the distal portion of the reusable applicator to deliver the first analyte sensor from the sensor carrier, moving the sharp carrier from the distal portion of the reusable applicator to the proximal portion of the reusable applicator and moving the actuator of the assembly to a third position after delivery of the first analyte sensor, and returning the actuator from the third position to the first position for receipt of another analyte sensor for delivery. The first position, the second position, and the third position are different. The reusable applicator can include a drive spring to move sensor carrier and the sharp carrier from the proximal portion to the distal portion. The reusable applicator can include a retraction spring to move the actuator to the third position.

According to certain embodiments, returning the actuator from the third position to the first position can include reloading, using the actuator of the assembly, the retraction spring by moving the sharp carrier from the proximal portion of the reusable applicator to the distal portion of the reusable applicator, and reloading the drive spring by moving the sensor carrier and the sharp carrier from the distal portion of the reusable applicator to the proximal portion of the reusable applicator.

The method can further comprise accessing the sharp carrier from the proximal portion of the reusable applicator for releasing the sharp module. The actuator can include a button and the method can comprise opening the button to access and remove the first sharp module when the actuator is in the third position.

According to embodiments of the present disclosure, the reusable applicator can include a latch to hold the sensor carrier at the proximal portion of the reusable applicator when the sensor carrier moves from the second position to the third position.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIGS. 3A to 3G are progressive views of an example embodiment of the assembly and application of the system of FIG. 1 incorporating a two-piece architecture.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is side view depicting an example embodiment of a housing.

FIG. 7B is a perspective view depicting an example embodiment of a distal end of a housing.

FIG. 7C is a side cross-sectional view depicting an example embodiment of a housing.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIG. 8F is a perspective view depicting an example embodiment of a compressible distal end of an applicator.

FIGS. 8G to 8K are cross-sectional views depicting example geometries for embodiments of compressible distal ends of an applicator.

FIG. 8L is a perspective view of an example embodiment of an applicator having a compressible distal end.

FIG. 8M is a cross-sectional view depicting an example embodiment of an applicator having a compressible distal end.

FIG. 9A is a proximal perspective view depicting an example embodiment of a sensor carrier.

FIG. 9B is a distal perspective view depicting an example embodiment of a sensor carrier.

FIG. 10 is a proximal perspective view of an example embodiment of a sharp carrier.

FIG. 11 is a side cross-section depicting an example embodiment of a sharp carrier.

FIGS. 12A to 12B are top and bottom perspective views, respectively, depicting an example embodiment of a sensor module.

FIGS. 13A and 13B are perspective and compressed views, respectively, depicting an example embodiment of a sensor connector.

FIG. 14 is a perspective view depicting an example embodiment of a sensor.

FIGS. 15A and 15B are bottom and top perspective views, respectively, of an example embodiment of a sensor module assembly.

FIGS. 16A and 16B are close-up partial views of an example embodiment of a sensor module assembly.

FIG. 17A is a perspective view depicting an example embodiment of a sharp module.

FIG. 17B is a perspective view depicting another example embodiment of a sharp module.

FIGS. 17C and 17D are a side view and a perspective view depicting another example embodiment of a sharp module.

FIG. 17E is a cross-sectional view depicting an example embodiment of an applicator.

FIG. 17F is a flow diagram depicting an example embodiment method for sterilizing an applicator assembly.

FIGS. 17G and 17H are photographs depicting example embodiments of sharp tips.

FIGS. 17I and 17J are perspective views depicting example embodiments of sharp modules.

FIG. 18A is a front view depicting an example embodiment of an applicator in accordance with the disclosed subject matter.

FIG. 18B is a cross-sectional view depicting various components of the applicator of FIG. 18A.

FIG. 19A is a cross-sectional view depicting an example embodiment of an applicator during a stage of deployment.

FIGS. 19B and 19C are perspective views, respectively, of an example embodiment of a sheath and a sensor carrier.

FIG. 19D is a cross-sectional view depicting an example embodiment of an applicator during a stage of deployment.

FIGS. 19E and 19F are perspective and close-up partial views, respectively, of an example embodiment of a sheath-sensor carrier assembly.

FIG. 19G is a cross-sectional view depicting an example embodiment of an applicator during a stage of deployment.

FIGS. 19H and 19I are close-up partial views of an example embodiment of a sheath-sensor carrier assembly.

FIG. 19J is a cross-sectional view depicting an example embodiment of an applicator during a stage of deployment.

FIGS. 19K and 19L are close-up partial views of an example embodiment of a sheath-sensor carrier assembly.

FIG. 19M is a front view depicting an example embodiment of an applicator during a stage of deployment.

FIG. 19N is a cross-sectional view depicting an example embodiment of an applicator during a stage of deployment.

FIG. 19O is a cross-sectional view depicting an example embodiment of an applicator during a stage of deployment.

FIGS. 19P and 19Q are perspective views of example embodiments of a disposable sensor carrier of a reusable powered applicator.

FIGS. 19R-1 and 19R-2 are perspective views of example embodiments of a disposable sensor carrier and a reusable applicator base of a reusable powered applicator.

FIGS. 19S to 19U are cross-sectional views depicting an example embodiment of a reusable powered applicator during various stages of operation.

FIG. 19V is a top-down view of an example embodiment of a reusable applicator base and disposable sensor carrier of a reusable powered applicator.

FIGS. 19W and 19X are a cross-sectional and a perspective view, respectively, or a reusable powered applicator in a ready-to-load state.

FIGS. 20A-20G depict an example embodiment of an applicator, where FIG. 20A is a front perspective view of the embodiment, FIG. 20B is a front side view of the embodiment, FIG. 20C is a rear side view of the embodiment, FIG. 20D is a left side view of the embodiment, FIG. 20E is a right side view of the embodiment, FIG. 20F is a top view of the embodiment, and FIG. 20G is a bottom view of the embodiment.

FIGS. 21A-21G depict another example embodiment of an applicator, where FIG. 21A is a front perspective view of the embodiment, FIG. 21B is a front side view of the embodiment, FIG. 21C is a rear side view of the embodiment, FIG. 21D is a left side view of the embodiment, FIG. 21E is a right side view of the embodiment, FIG. 21F is a top view of the embodiment, and FIG. 21G is a bottom view of the embodiment.

FIGS. 21H-I are enlarged cross-sectional side views of the interface between applicator housing and applicator cap of an example embodiment of an applicator.

FIGS. 21J-K are enlarged cross-sectional side views of applicator housing and applicator cap.

FIGS. 22A-22G depict an example embodiment of a sensor control device, where FIG. 22A is a front perspective view of the embodiment, FIG. 22B is a front side view of the embodiment, FIG. 22C is a rear side view of the embodiment, FIG. 22D is a left side view of the embodiment, FIG. 22E is a right side view of the embodiment, FIG. 22F is a top view of the embodiment, and FIG. 22G is a bottom view of the embodiment.

FIGS. 23A-23G depict another example embodiment of a sensor control device, where FIG. 23A is a front perspective view of the embodiment, FIG. 23B is a front side view of the embodiment, FIG. 23C is a rear side view of the embodiment, FIG. 23D is a left side view of the embodiment, FIG. 23E is a right side view of the embodiment, FIG. 23F is a top view of the embodiment, and FIG. 23G is a bottom view of the embodiment.

FIGS. 24A-24G depict another example embodiment of a sensor control device, where FIG. 24A is a front perspective view of the embodiment, FIG. 24B is a front side view of the embodiment, FIG. 24C is a rear side view of the embodiment, FIG. 24D is a left side view of the embodiment, FIG. 24E is a right side view of the embodiment, FIG. 24F is a top view of the embodiment, and FIG. 24G is a bottom view of the embodiment.

FIGS. 25A-25G depict another example embodiment of a sensor control device, where FIG. 25A is a front perspective view of the embodiment, FIG. 25B is a front side view of the embodiment, FIG. 25C is a rear side view of the embodiment, FIG. 25D is a left side view of the embodiment, FIG. 25E is a right side view of the embodiment, FIG. 25F is a top view of the embodiment, and FIG. 25G is a bottom view of the embodiment.

FIGS. 26A-26G depict another example embodiment of a sensor control device, where FIG. 26A is a front perspective view of the embodiment, FIG. 26B is a front side view of the embodiment, FIG. 26C is a rear side view of the embodiment, FIG. 26D is a left side view of the embodiment, FIG. 26E is a right side view of the embodiment, FIG. 26F is a top view of the embodiment, and FIG. 26G is a bottom view of the embodiment.

FIGS. 27A-27G depict another example embodiment of a sensor control device, where FIG. 27A is a front perspective view of the embodiment, FIG. 27B is a front side view of the embodiment, FIG. 27C is a rear side view of the embodiment, FIG. 27D is a left side view of the embodiment, FIG. 27E is a right side view of the embodiment, FIG. 27F is a top view of the embodiment, and FIG. 27G is a bottom view of the embodiment.

FIGS. 28A-28G depict another example embodiment of a sensor control device, where FIG. 28A is a front perspective view of the embodiment, FIG. 28B is a front side view of the embodiment, FIG. 28C is a rear side view of the embodiment, FIG. 28D is a left side view of the embodiment, FIG. 28E is a right side view of the embodiment, FIG. 28F is a top view of the embodiment, and FIG. 28G is a bottom view of the embodiment.

FIGS. 29A-29G depict another example embodiment of a sensor control device, where FIG. 29A is a front perspective view of the embodiment, FIG. 29B is a front side view of the embodiment, FIG. 29C is a rear side view of the embodiment, FIG. 29D is a left side view of the embodiment, FIG. 29E is a right side view of the embodiment, FIG. 29F is a top view of the embodiment, and FIG. 29G is a bottom view of the embodiment.

FIGS. 30A-30G depict an example embodiment of an applicator, where FIG. 30A is a front perspective view of the embodiment, FIG. 30B is a front side view of the embodiment, FIG. 30C is a rear side view of the embodiment, FIG. 30D is a left side view of the embodiment, FIG. 30E is a right side view of the embodiment, FIG. 30F is a top view of the embodiment, and FIG. 30G is a bottom view of the embodiment.

FIGS. 31A-31G depict another example embodiment of an applicator, where FIG. 31A is a front perspective view of the embodiment, FIG. 31B is a front side view of the embodiment, FIG. 31C is a rear side view of the embodiment, FIG. 31D is a left side view of the embodiment, FIG. 31E is a right side view of the embodiment, FIG. 31F is a top view of the embodiment, and FIG. 31G is a bottom view of the embodiment.

FIGS. 32A-32G depict an example embodiment of a sensor control device, where FIG. 32A is a front perspective view of the embodiment, FIG. 32B is a front side view of the embodiment, FIG. 32C is a rear side view of the embodiment, FIG. 32D is a left side view of the embodiment, FIG. 32E is a right side view of the embodiment, FIG. 32F is a top view of the embodiment, and FIG. 32G is a bottom view of the embodiment.

FIGS. 33A-33G depict another example embodiment of a sensor control device, where FIG. 33A is a front perspective view of the embodiment, FIG. 33B is a front side view of the embodiment, FIG. 33C is a rear side view of the embodiment, FIG. 33D is a left side view of the embodiment, FIG. 33E is a right side view of the embodiment, FIG. 33F is a top view of the embodiment, and FIG. 33G is a bottom view of the embodiment.

FIGS. 34A-34G depict another example embodiment of a sensor control device, where FIG. 34A is a front perspective view of the embodiment, FIG. 34B is a front side view of the embodiment, FIG. 34C is a rear side view of the embodiment, FIG. 34D is a left side view of the embodiment, FIG. 34E is a right side view of the embodiment, FIG. 34F is a top view of the embodiment, and FIG. 34G is a bottom view of the embodiment.

FIGS. 35A-35G depict another example embodiment of a sensor control device, where FIG. 35A is a front perspective view of the embodiment, FIG. 35B is a front side view of the embodiment, FIG. 35C is a rear side view of the embodiment, FIG. 35D is a left side view of the embodiment, FIG. 35E is a right side view of the embodiment, FIG. 35F is a top view of the embodiment, and FIG. 35G is a bottom view of the embodiment.

FIG. 36 is a chart reflecting certain characteristics of example embodiments of materials and seals used for packaging.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for the improved assembly and use of analyte sensor insertion devices for use with in vivo analyte monitoring systems. In particular, several embodiments of the present disclosure are designed to improve the method of sensor insertion with respect to in vivo analyte monitoring systems and, in particular, to minimize trauma to an insertion site during a sensor insertion process. Some embodiments, for example, include a powered sensor insertion mechanism configured to operate at a higher, controlled speed relative to a manual insertion mechanism, in order to reduce trauma to an insertion site. In other embodiments, an applicator having a compressible distal end can stretch and flatten the skin surface at the insertion site, and consequently, can reduce the likelihood of a failed insertion as a result of skin tenting. In still other embodiments, a sharp with an offset tip, or a sharp manufactured utilizing a plastic material or a coined manufacturing process can also reduce trauma to an insertion site. In sum, these embodiments can improve the likelihood of a successful sensor insertion and reduce the amount of trauma at the insertion site, to name a few advantages.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

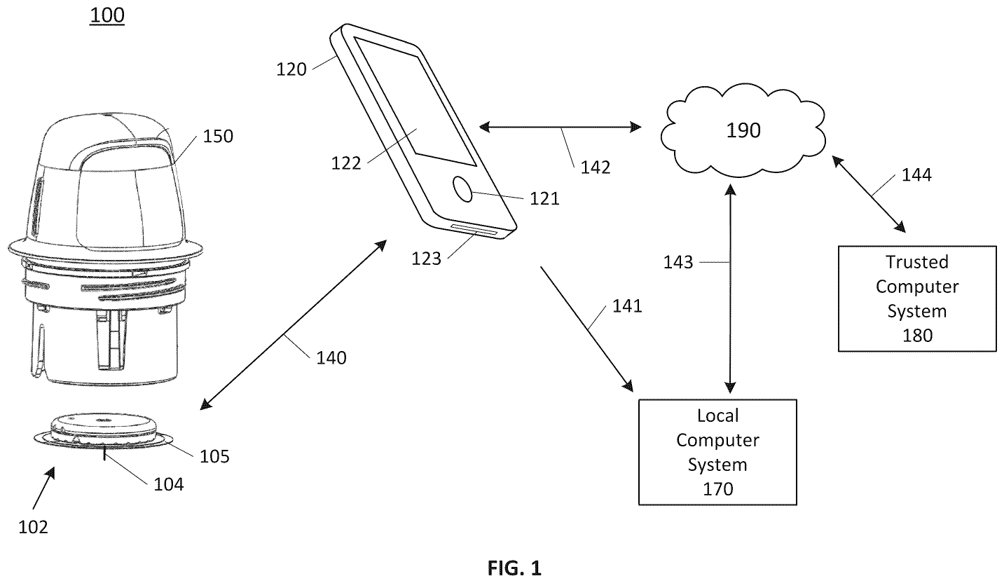

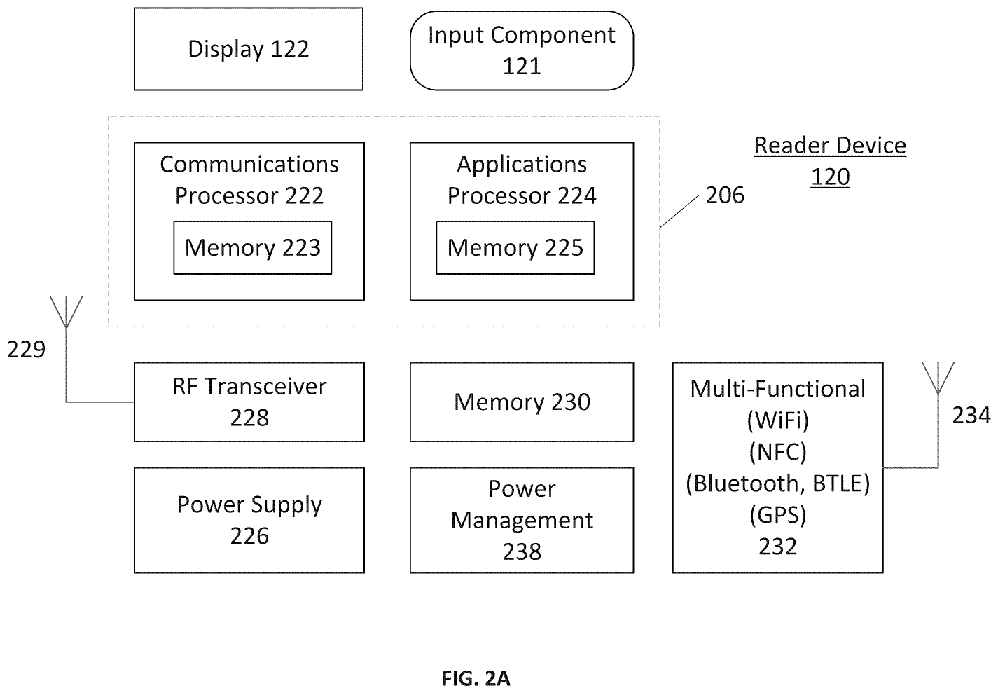

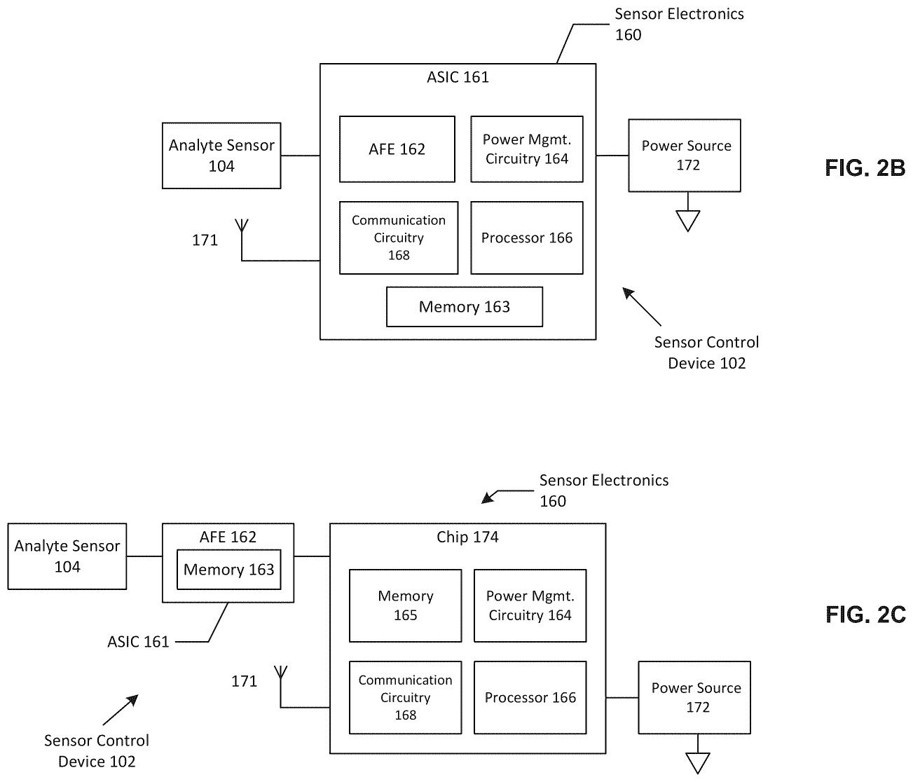

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121, and the device battery can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiment of Reader Device

FIG. 2A is a block diagram depicting an example embodiment of a reader device 120 configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Assembly Processes for Sensor Control Device

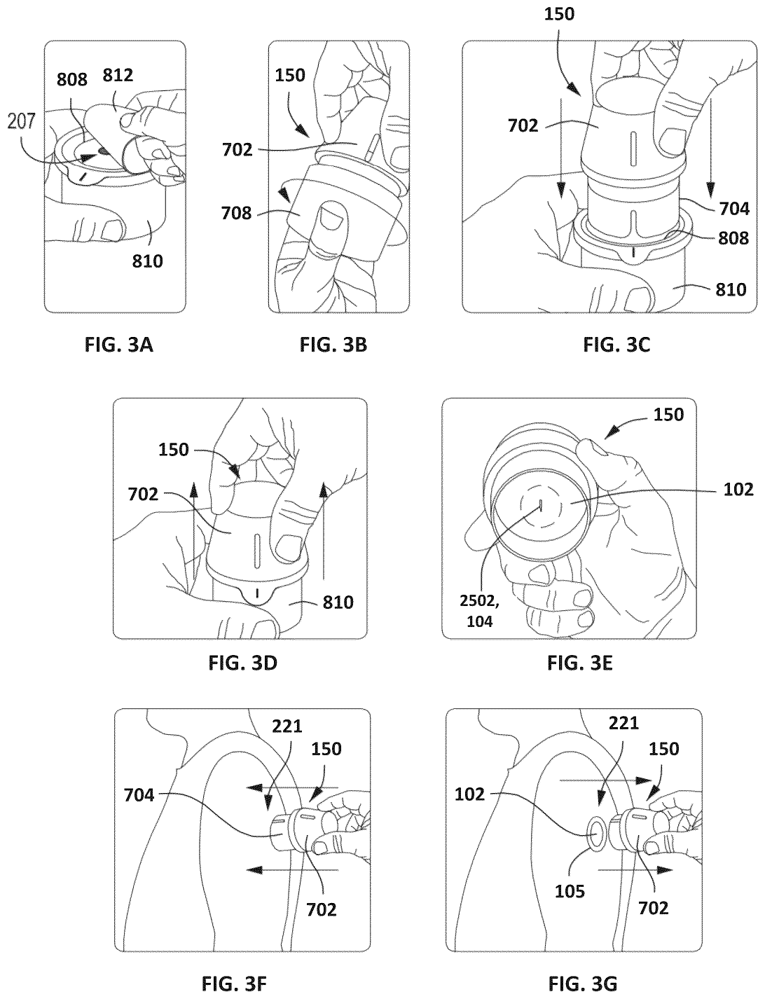

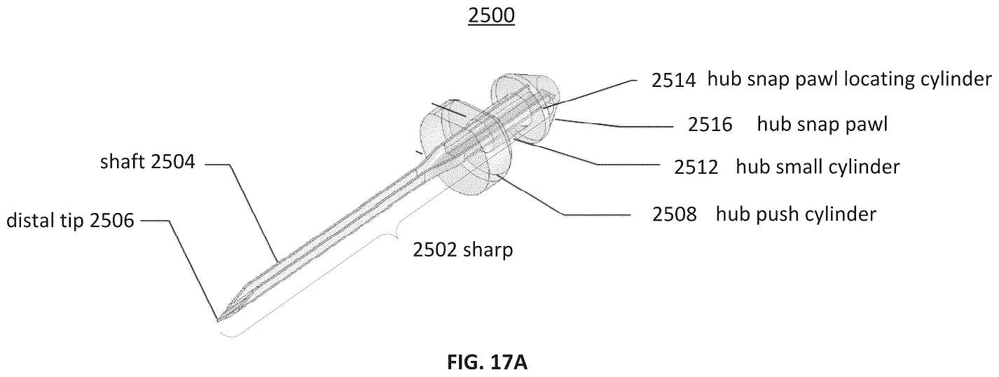

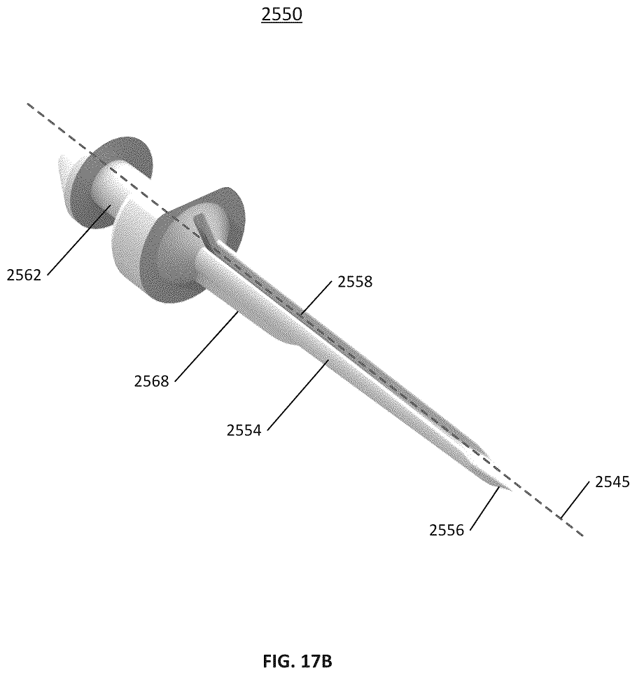

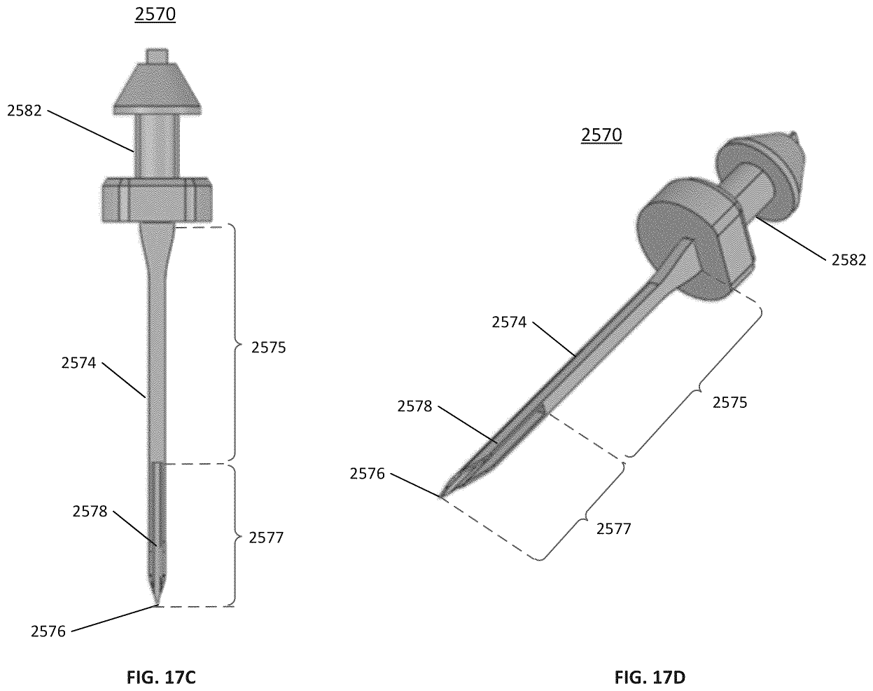

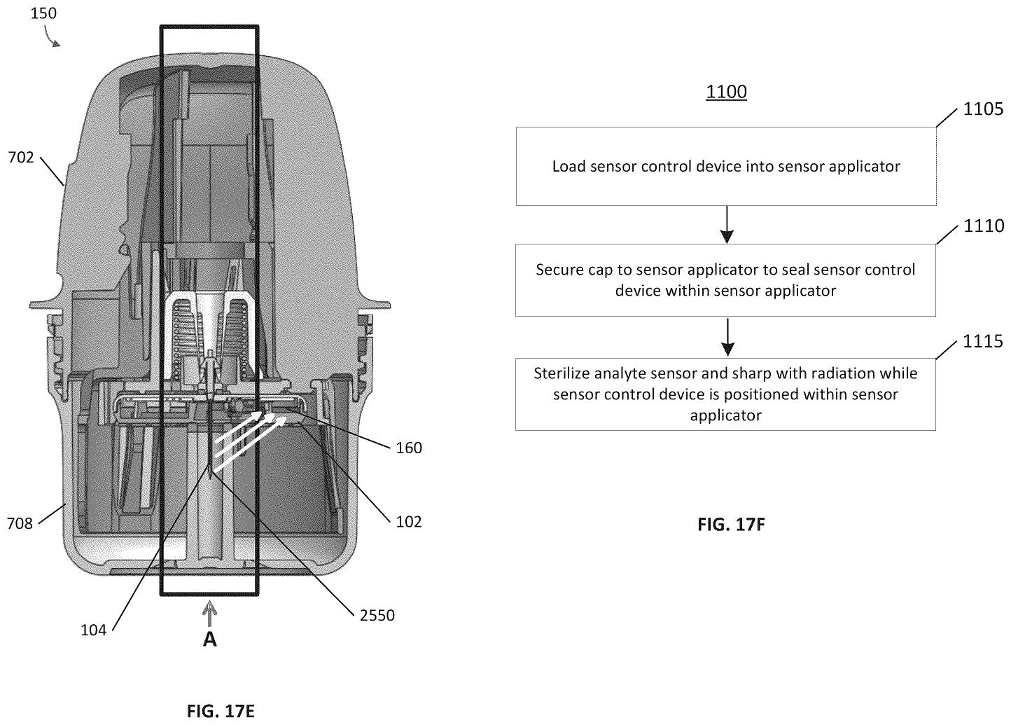

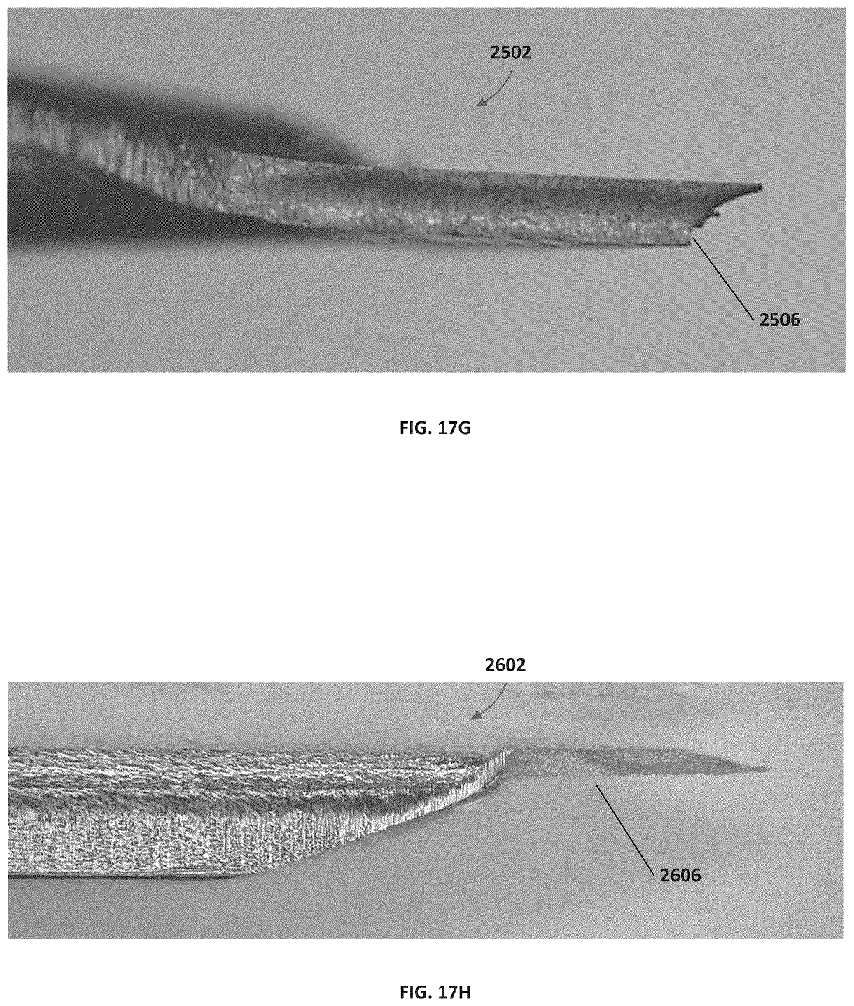

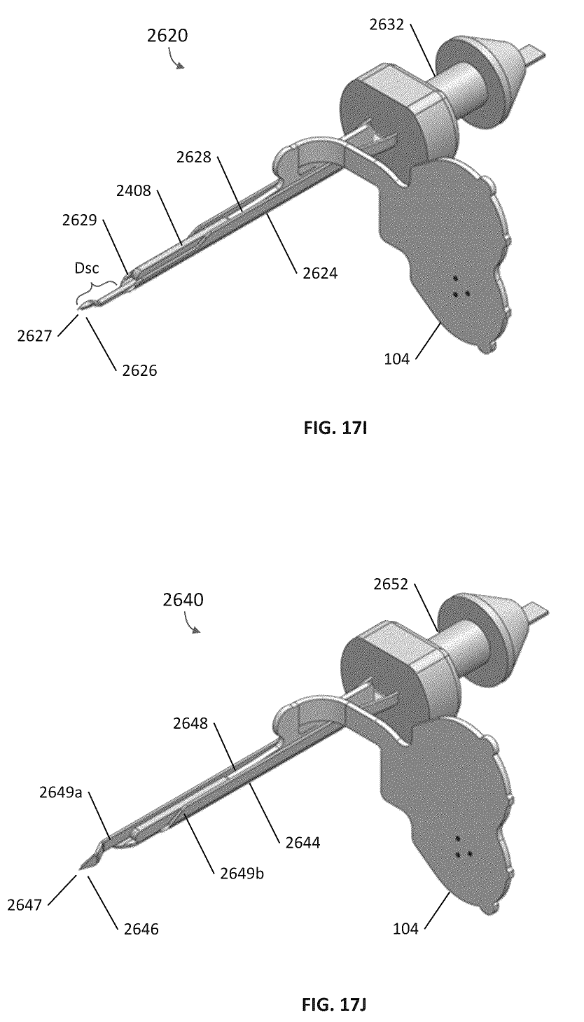

According to some embodiments, the components of sensor control device 102 can be acquired by a user in multiple packages requiring final assembly by the user before delivery to an appropriate user location. FIGS. 3A-3E depict an example embodiment of an assembly process for sensor control device 102 by a user, including preparation of separate components before coupling the components in order to ready the sensor for delivery. In other embodiments, such as those described with respect to FIGS. 17B to 17F, components of the sensor control device 102 and applicator 150 can be acquired by a user in a single package. FIGS. 3F-3G depict an example embodiment of delivery of sensor control device 102 to an appropriate user location by selecting the appropriate delivery location and applying device 102 to the location.

FIG. 3A depicts a sensor container or tray 810 that has a removable lid 812. The user prepares the sensor tray 810 by removing the lid 812, which acts as a sterile barrier to protect the internal contents of the sensor tray 810 and otherwise maintain a sterile internal environment. Removing the lid 812 exposes a platform 808 positioned within the sensor tray 810, and a plug assembly 207 (partially visible) is arranged within and otherwise strategically embedded within the platform 808. The plug assembly 207 includes a sensor module (not shown) and a sharp module (not shown). The sensor module carries the sensor 104 (FIG. 1), and the sharp module carries an associated sharp used to help deliver the sensor 104 transcutaneously under the user's skin during application of the sensor control device 102 (FIG. 1).

FIG. 3B depicts the sensor applicator 150 and the user preparing the sensor applicator 150 for final assembly. The sensor applicator 150 includes a housing 702 sealed at one end with an applicator cap 708. In some embodiments, for example, an O-ring or another type of sealing gasket may seal an interface between the housing 702 and the applicator cap 708. In at least one embodiment, the O-ring or sealing gasket may be molded onto one of the housing 702 and the applicator cap 708. The applicator cap 708 provides a barrier that protects the internal contents of the sensor applicator 150. In particular, the sensor applicator 150 contains an electronics housing (not shown) that retains the electrical components for the sensor control device 102 (FIG. 1), and the applicator cap 708 may or may not maintain a sterile environment for the electrical components. Preparation of the sensor applicator 150 includes uncoupling the housing 702 from the applicator cap 708, which can be accomplished by unscrewing the applicator cap from the housing 702. The applicator cap 708 can then be discarded or otherwise placed aside.

FIG. 3C depicts the user inserting the sensor applicator 150 into the sensor tray 810. The sensor applicator 150 includes a sheath 704 configured to be received by the platform 808 to temporarily unlock the sheath 704 relative to the housing 702, and also temporarily unlock the platform 808 relative to the sensor tray 810. Advancing the housing 702 into the sensor tray 810 results in the plug assembly 207 (FIG. 3A) arranged within the sensor tray 810, including the sensor and sharp modules, being coupled to the electronics housing arranged within the sensor applicator 150.

In FIG. 3D, the user removes the sensor applicator 150 from the sensor tray 810 by proximally retracting the housing 702 with respect to the sensor tray 810.

FIG. 3E depicts the bottom or interior of the sensor applicator 150 following removal from the sensor tray 810 (FIGS. 3A and 3C). The sensor applicator 150 is removed from the sensor tray 810 with the sensor control device 102 fully assembled therein and positioned for delivery to the target monitoring location. As illustrated, a sharp 2502 extends from the bottom of the sensor control device 102 and carries a portion of the sensor 104 within a hollow or recessed portion thereof. The sharp 2502 is configured to penetrate the skin of a user and thereby place the sensor 104 into contact with bodily fluid.

FIGS. 3F and 3G depict example delivery of the sensor control device 102 to a target monitoring location 221, such as the back of an arm of the user. FIG. 3F shows the user advancing the sensor applicator 150 toward the target monitoring location 221. Upon engaging the skin at the target monitoring location 221, the sheath 704 collapses into the housing 702, which allows the sensor control device 102 (FIGS. 3E and 3G) to advance into engagement with the skin. With the help of the sharp 2502 (FIG. 3E), the sensor 104 (FIG. 3E) is advanced transcutaneously into the patient's skin at the target monitoring location 221.

FIG. 3G shows the user retracting the sensor applicator 150 from the target monitoring location 221, with the sensor control device 102 successfully attached to the user's skin. The adhesive patch 105 (FIG. 1) applied to the bottom of sensor control device 102 adheres to the skin to secure the sensor control device 102 in place. The sharp 2502 (FIG. 3E) is automatically retracted when the housing 702 is fully advanced at the target monitoring location 221, while the sensor 104 (FIG. 3E) is left in position to measure analyte levels.

According to some embodiments, system 100, as described with respect to FIGS. 3A-3G and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes.

### Example Embodiment of Sensor Applicator Device

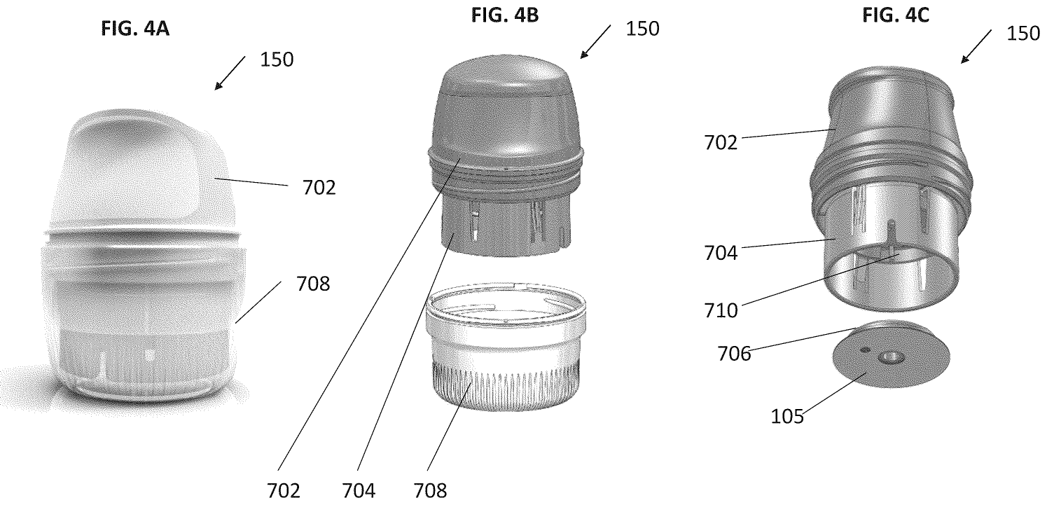

FIG. 4A is a side view depicting an example embodiment of an applicator device 150 coupled with screw cap 708. This is one example of how applicator 150 is shipped to and received by a user, prior to assembly by the user with a sensor. In other embodiments, applicator 150 can be shipped to the user with the sensor and sharp contained therein. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within sensor carrier 710 of sheath 704, when cap 708 is in place.

### Example Embodiment of Tray and Sensor Module Assembly

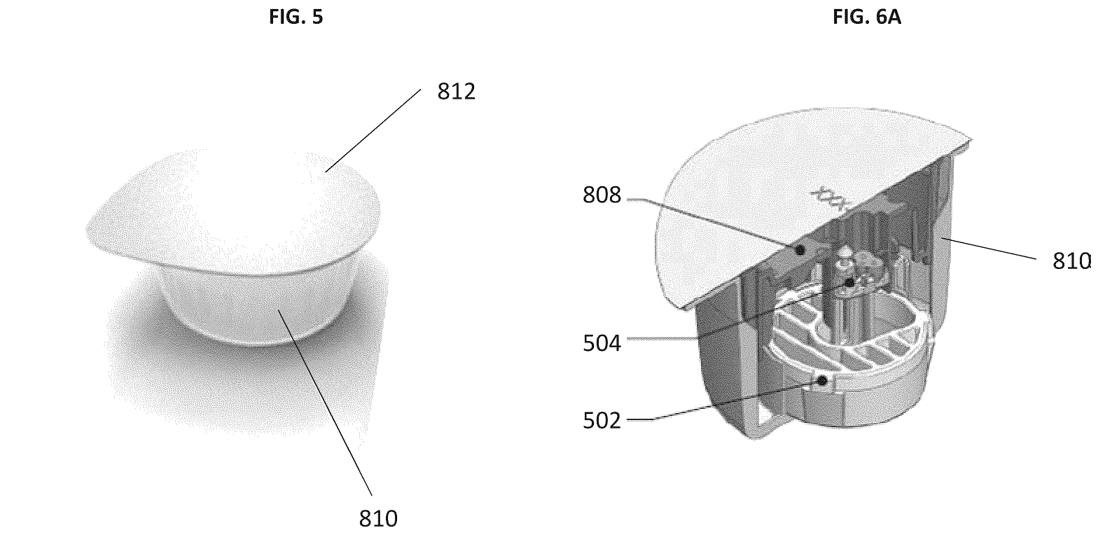

FIG. 5 is a proximal perspective view depicting an example embodiment of a tray 810 with sterilization lid 812 removably coupled thereto, which, in some embodiments, may be representative of how the package is shipped to and received by a user prior to assembly.

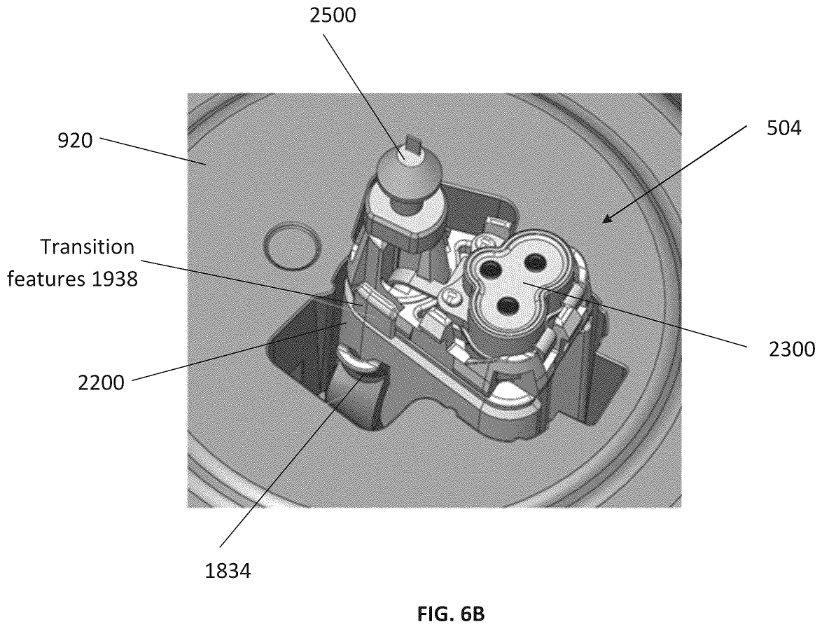

FIG. 6A is a proximal perspective, cutaway view depicting sensor delivery components within tray 810, according to some embodiments. Platform 808 is slidably coupled within tray 810. Desiccant 502 is stationary with respect to tray 810. Sensor module 504 is mounted within tray 810.

FIG. 6B is a proximal perspective view depicting an example embodiment of a sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

### Example Embodiment of Applicator Housing

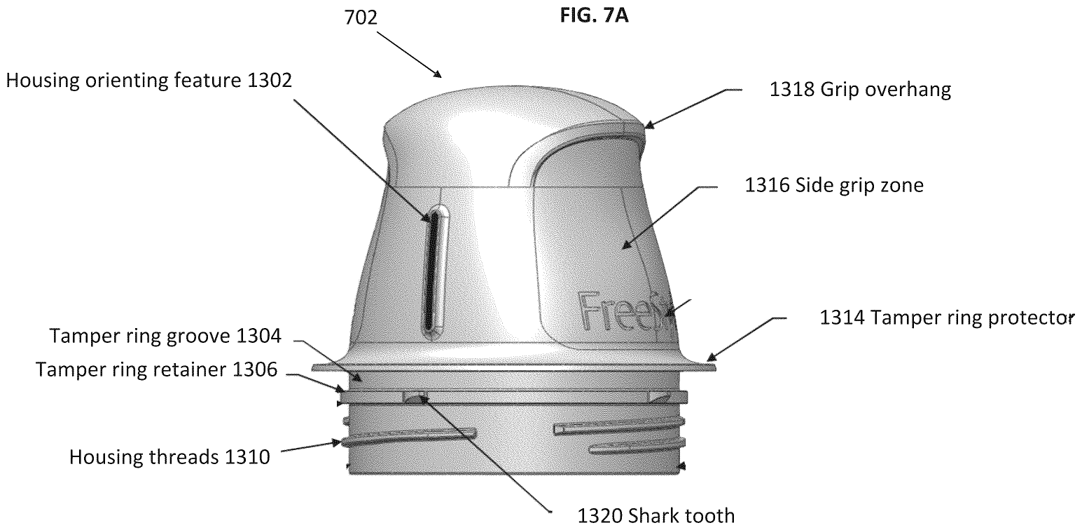

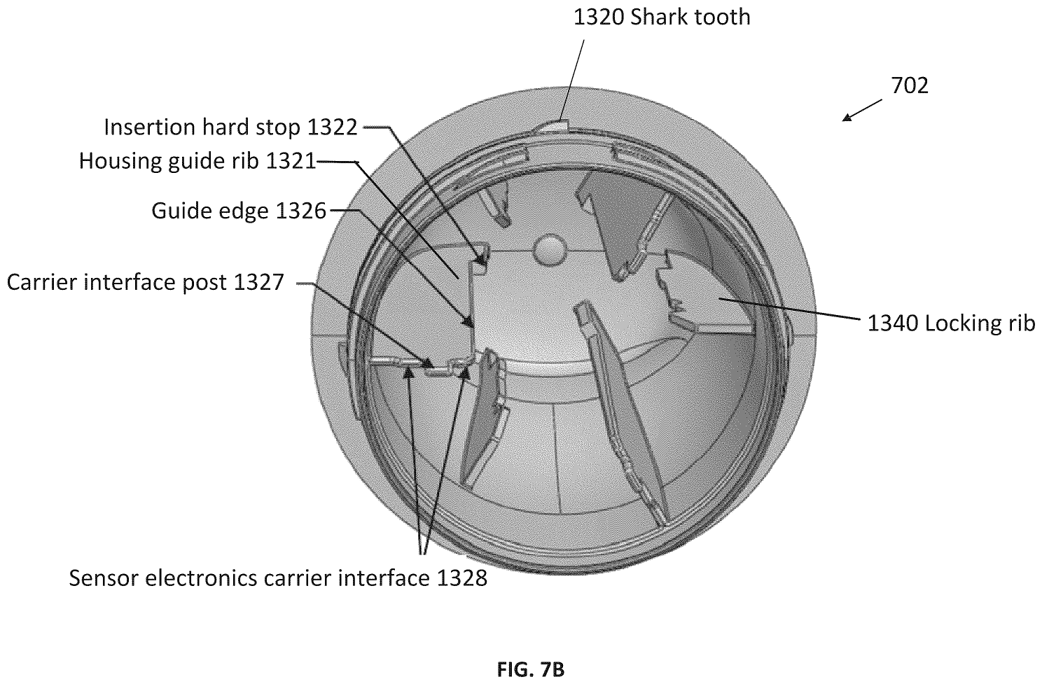

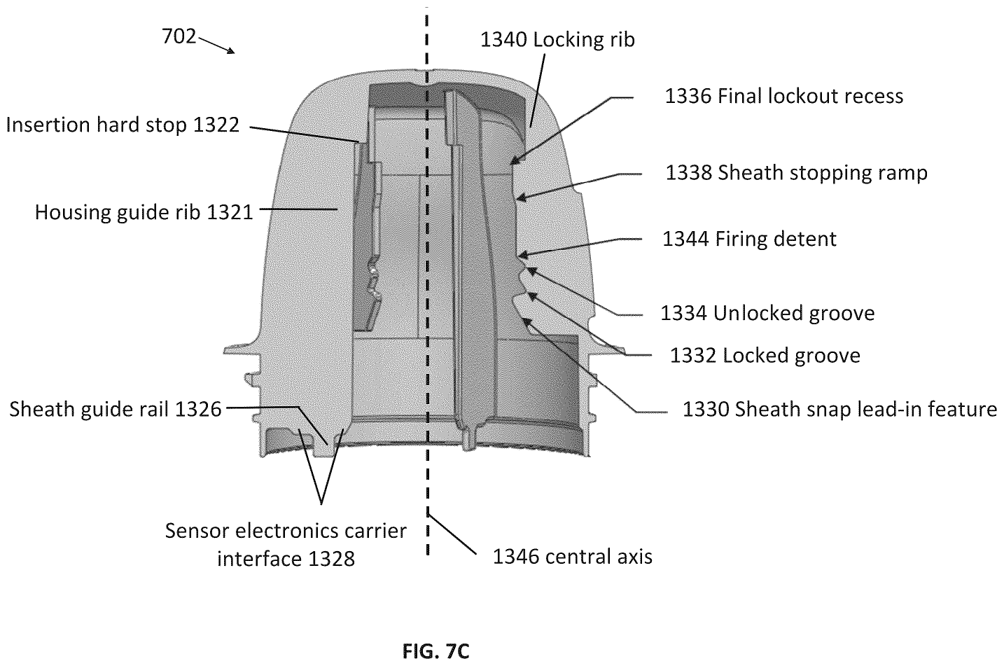

FIG. 7A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in ease of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

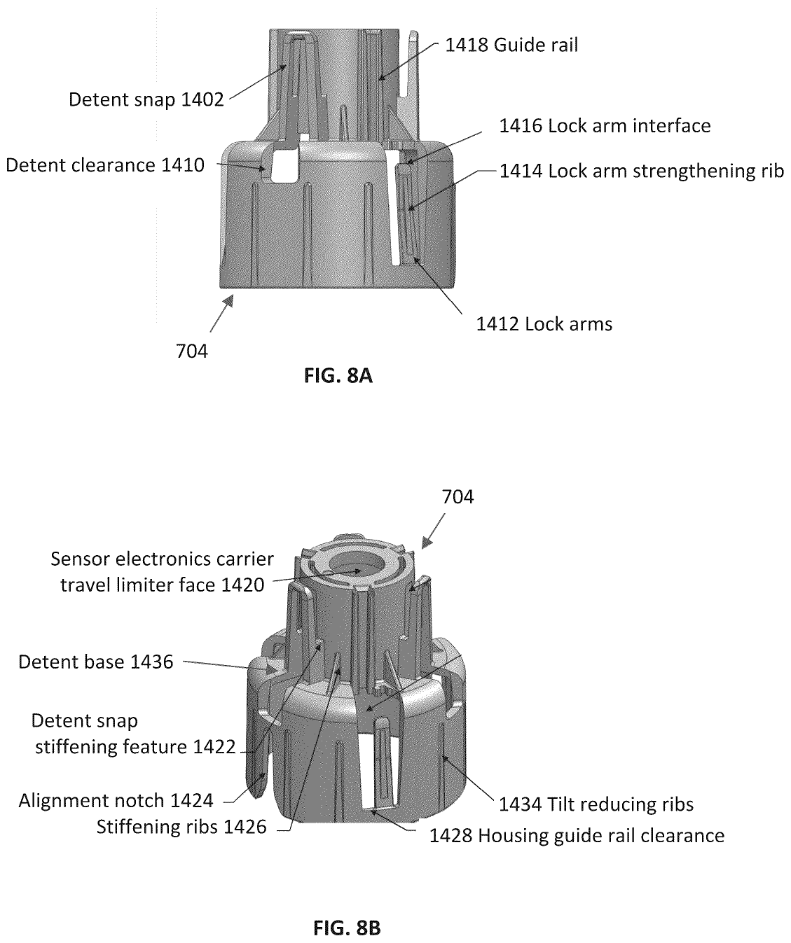

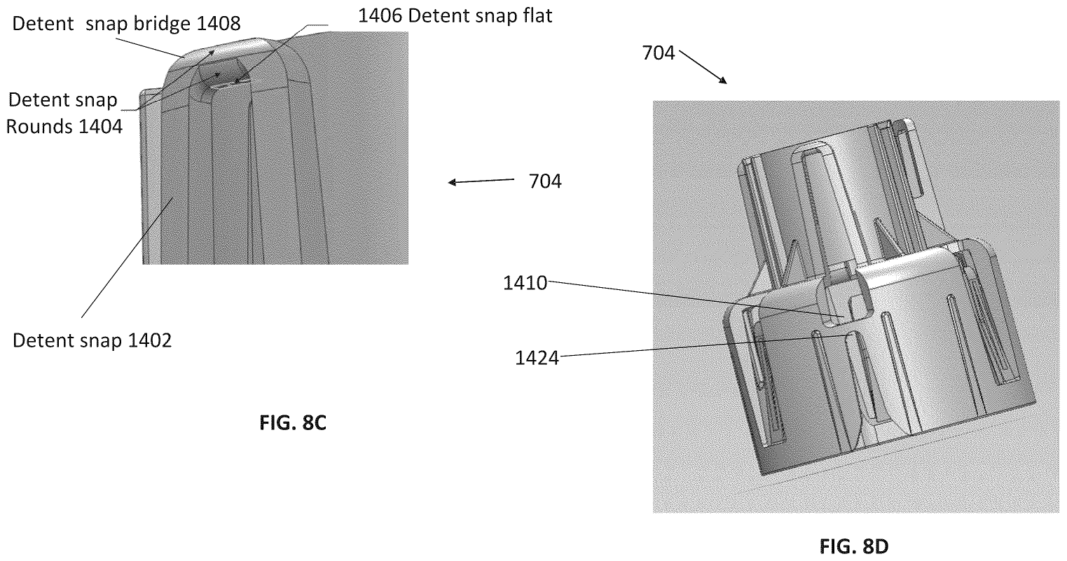

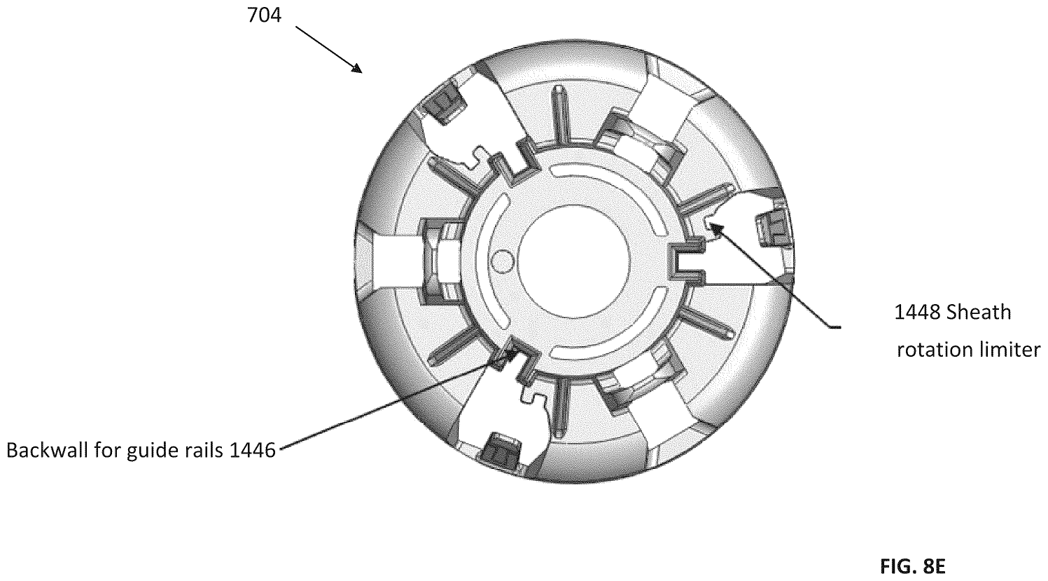

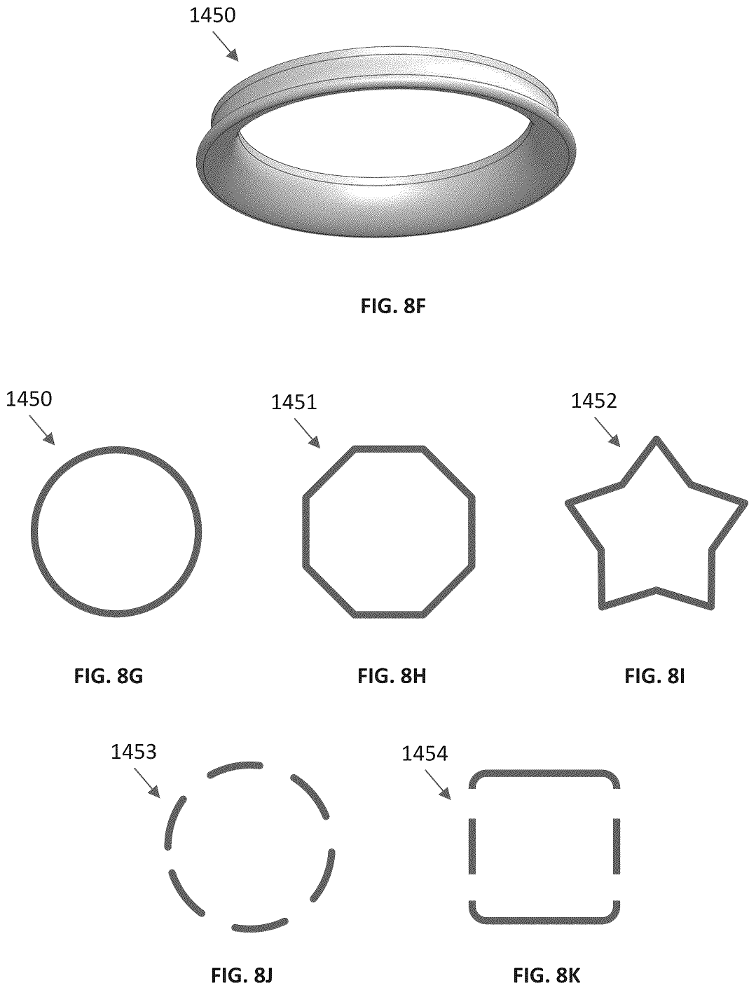

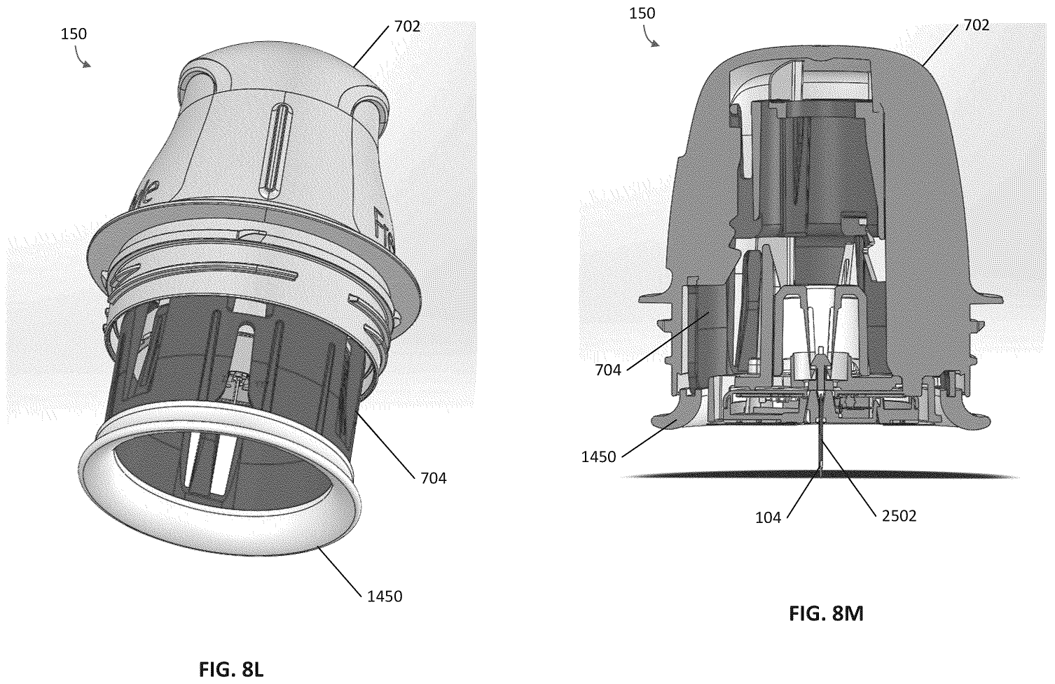

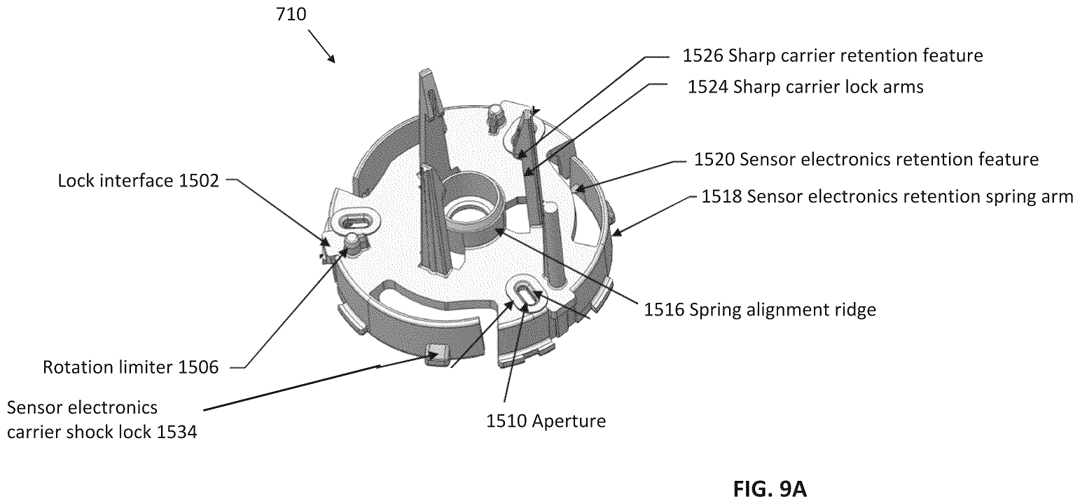

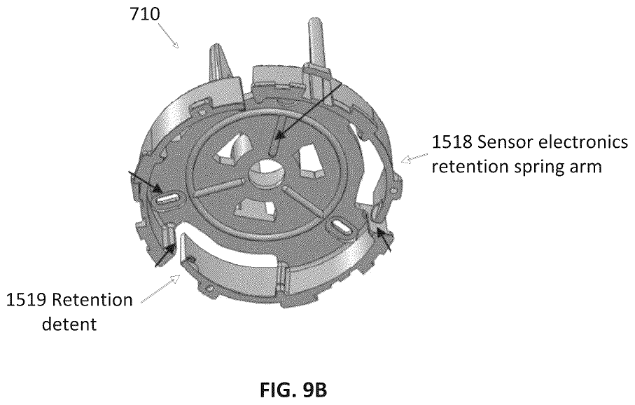

FIG. 7B is a perspective view depicting a distal end of housing 702. Here, three housing guide structures (or “guide ribs”) 1321 are located at 120 degree angles with respect to each other, and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a sensor carrier travel limiter face 1420 of a sheath 704 (FIG. 8B) to abut during use, preventing sensor carrier travel limiter face 1420 from moving any further in a proximal direction. A carrier interface post 1327 passes through an aperture 1510 (FIG. 9A) of sensor carrier 710 during an assembly. A sensor carrier interface 1328 can be a rounded, distally facing surface of housing guide ribs 1321 which interfaces with sensor carrier 710.

FIG. 7C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor carrier travel limiter face 1420.

### Example Embodiment of Applicator Sheath

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock sensor carrier 710 to sheath 704 when lock arm interface 1416 of lock arms 1412 engage lock interface 1502 of sensor carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704.

FIG. 8F is a perspective view depicting an example embodiment of a compressible distal end 1450, which can be attached and/or detached from a sheath 704 of an applicator 150. In a general sense, the embodiments described herein operate by flattening and stretching a skin surface at a predetermined site for sensor insertion. Moreover, the embodiments described herein may also be utilized for other medical applications, such as, e.g., transdermal drug delivery, needle injection, wound closure stitches, device implantation, the application of an adhesive surface to the skin, and other like applications.

By way of background, those of skill the art will appreciate that skin is a highly anisotropic tissue from a biomechanical standpoint and varies largely between individuals. This can affect the degree to which communication between the underlying tissue and the surrounding environment can be performed, e.g., with respect to drug diffusion rates, the ability to penetrate skin with a sharp, or sensor insertion into the body at a sharp-guided insertion site.

In particular, the embodiments described herein are directed to reducing the anisotropic nature of the skin in a predetermined area by flattening and stretching the skin, and thereby improving upon the aforementioned applications. Smoothing the skin (e.g., flattening to remove wrinkles) before mating with a similarly shaped (e.g., a flat, round adhesive pad of a sensor control unit) can produce a more consistent surface area contact interface. As the surface profile of the skin approaches the profile specifications of the designed surface of the device (or, e.g., the designed area of contact for drug delivery), the more consistent contact (or drug dosing) can be achieved. This can also be advantageous with respect to wearable adhesives by creating a continuum of adhesive-to-skin contact in a predetermined area without wrinkles. Other advantages can include (1) an increased wear duration for devices that rely on skin adhesion for functionality, and (2) a more predictable skin contact area, which would improve dosing in transcutaneous drug/pharmaceutical delivery.

In addition, skin flattening (e.g., as a result of tissue compression) combined with stretching can reduce the skin's viscoelastic nature and increase its rigidity which, in turn, can increase the success rate of sharp-dependent sensor placement and functionality.

With respect to sensor insertion, puncture wounds can contribute to early signal aberration (ESA) in sensors and may be mitigated when the skin has been flattened and stretched rigid. Some known methods to minimize a puncture wound include: (1) reducing the introducers' size, or (2) limiting the length of the needle inserted into the body. However, these known methods may reduce the insertion success rate due to the compliance of the skin. For example, when a sharp tip touches the skin, before the tip penetrates the skin, the skin deforms inward into the body, a phenomenon also referred to as “skin tenting.” If the sharp is not stiff enough due to a smaller cross-sectional area and/or not long enough, the sharp may fail to create an insertion point large enough, or in the desired location due to deflection, for the sensor to pass through the skin and be positioned properly. The degree of skin tenting can vary between and within subjects, meaning the distance between a sharp and a skin surface can vary between insertion instances. Reducing this variation by stretching and flattening the skin can allow for a more accurately functioning and consistent sensor insertion mechanism.

Referring to FIG. 8F, a perspective view depicts an example embodiment of a compressible distal end 1450 of an applicator 150. According to some embodiments, compressible distal end 1450 can be manufactured from an elastomeric material. In other embodiments, compressible distal end 1450 can be made of metal, plastic, composite legs or springs, or a combination thereof.

In some embodiments, compressible distal end 1450 can be detachable from an applicator 150 and used with various other similar or dissimilar applicators or medical devices. In other embodiments, compressible distal end 1450 can be manufactured as part of the sheath 704. In still other embodiments, the compressible distal end 1450 can be attached to other portions of applicator 150 (e.g., sensor carrier), or, alternatively, can be used as a separate standalone device. Furthermore, although compressible distal end 1450 is shown in FIGS. 8F and 8G as having a continuous ring geometry, other configurations can be utilized. For example, FIGS. 8H to 8K are cross-sectional views depicting various example compressible distal ends, having an octagonal geometry 1451 (FIG. 8H), star-shaped geometry 1452 (FIG. 8I), a non-continuous ring geometry 1453 (FIG. 8J), and a non-continuous rectangular geometry (FIG. 8K). With respect to FIGS. 8J and 8K, a compressible distal end with a non-continuous geometry would have a plurality of points or spans to contact the predetermined area of skin. Those of skill in the art will recognize that other geometries are possible and fully within the scope of the present disclosure.

FIGS. 8L and 8M are a perspective view and a cross-sectional view, respectively, depicting an applicator 150 having a compressible distal end 1450. As shown in FIGS. 8L and 8M, applicator 150 can also include applicator housing 702, sheath 704 to which compressible distal end 1450 is attached, sharp 2502, and sensor 104.

According to some embodiments, in operation, the compressible distal end 1450 of applicator is first positioned on a skin surface of the subject. The subject then applies a force on the applicator, e.g., in a distal direction, which causes compressible distal end 1450 to stretch and flatten the portion of the skin surface beneath. In some embodiments, for example, compressible distal end 1450 can be comprised of an elastomeric material and biased in a radially inward direction. In other embodiments, compressible distal end 1450 can be biased in a radially outward direction. The force on the applicator can cause an edge portion of the compressible distal end 1450 in contact with the skin surface to be displaced in a radially outward direction, creating radially outward forces on the portion of the skin surface beneath the applicator, and causing the skin surface to be stretched and flattened.

Furthermore, according to some embodiments, applying the force on the applicator also causes a medical device, such as a sensor control unit, to advance from a first position within the applicator to a second position adjacent to the skin surface. According to one aspect of some embodiments, the compressible distal end 1450 can be in an unloaded state in the first position (e.g., before the force is applied on the applicator), and a loaded state in the second position (e.g., after the force is applied on the applicator). Subsequently, the medical device is applied to the stretched and flattened portion of the skin surface beneath the compressible distal end 1450. According to some embodiments, the application of the medical device can include placing an adhesive surface 105 of a sensor control unit 102 on the skin surface and/or positioning at least a portion of an analyte sensor under the skin surface. The analyte sensor can be an in vivo analyte sensor configured to measure an analyte level in a bodily fluid of the subject. In still other embodiments, the application of the medical device can include placing a drug-loaded patch on the skin surface. Those of skill in the art will appreciate that a compressible distal end can be utilized with any of the aforementioned medical applications and is not meant to be limited to use in an applicator for analyte sensor insertion.

### Example Embodiments of Sensor Carriers

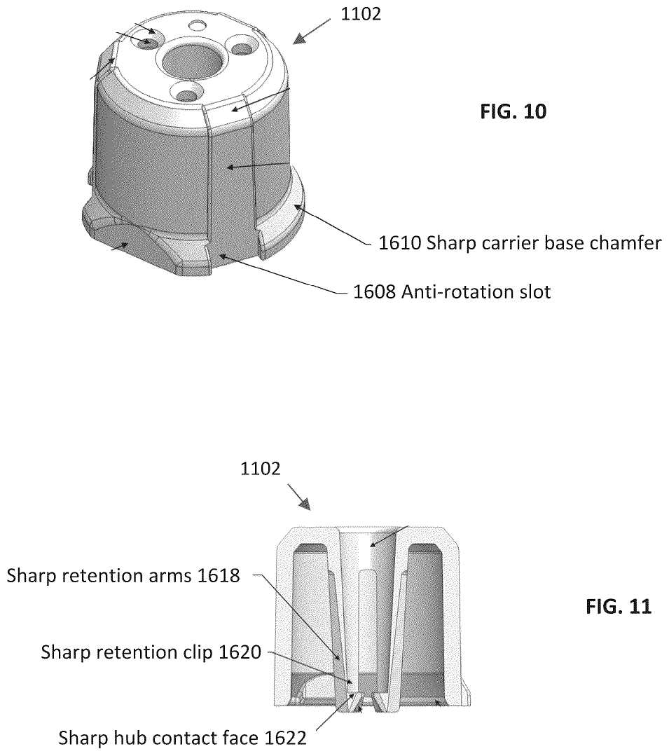

FIG. 9A is a proximal perspective view depicting an example embodiment of sensor carrier 710 that can retain sensor electronics within applicator 150. It can also retain sharp carrier 1102 with sharp module 2500. In this example embodiment, sensor carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of sensor carrier 710 extending outward and can lock sensor carrier 710 for added safety prior to firing. Rotation limiter 1506 can be a proximally extending relatively short protrusion on a proximal surface of sensor carrier 710 which limits rotation of carrier 710. Sharp carrier lock arms 1524 can interface with sharp carrier 1102 as described with reference to FIGS. 10 and 11 below.

FIG. 9B is a distal perspective view of sensor carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

### Example Embodiments of Sharp Carriers

FIGS. 10 and 11 are a proximal perspective view and a side cross-sectional view, respectively, depicting an example embodiment of sharp carrier 1102. Sharp carrier 1102 can grasp and retain sharp module 2500 within applicator 150. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

As shown in FIG. 11, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 17A).

### Example Embodiments of Sensor Modules

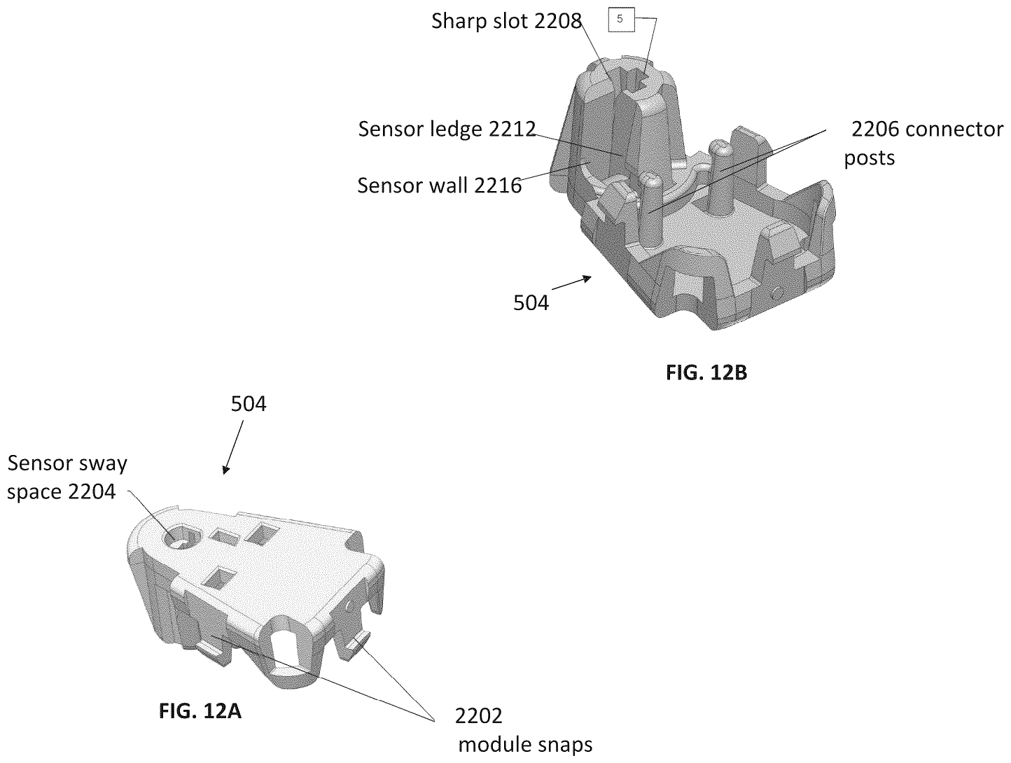

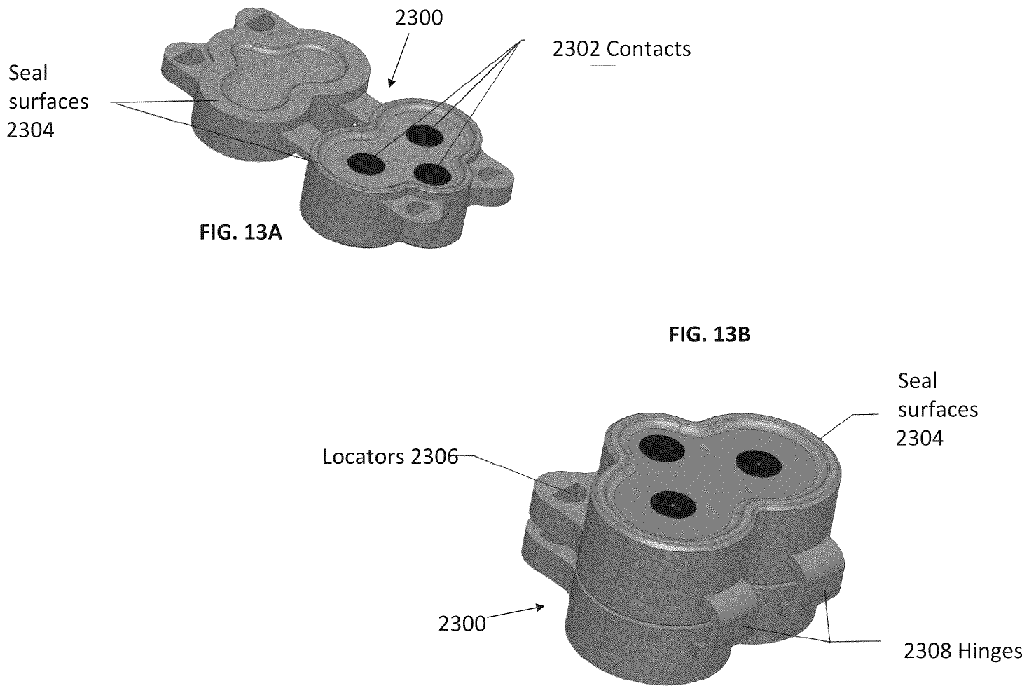

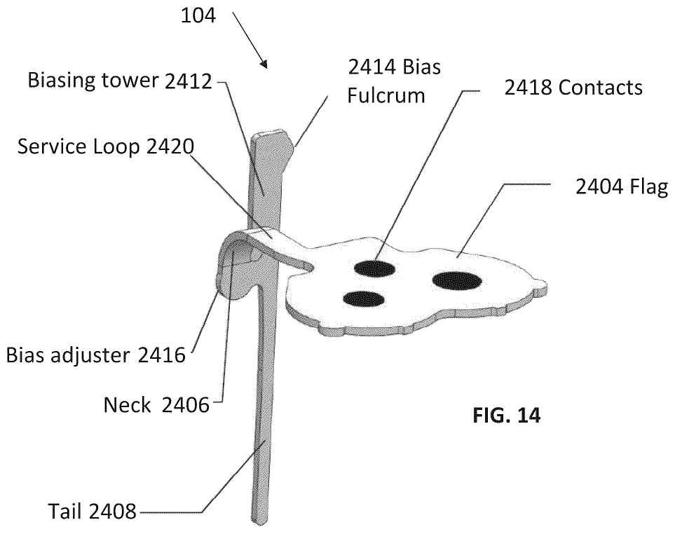

FIGS. 12A and 12B are a top perspective view and a bottom perspective view, respectively, depicting an example embodiment of sensor module 504. Module 504 can hold a connector 2300 (FIGS. 13A and 13B) and a sensor 104 (FIG. 14). Module 504 is capable of being securely coupled with electronics housing 706. One or more deflectable arms or module snaps 2202 can snap into the corresponding features 2010 of housing 706. A sharp slot 2208 can provide a location for sharp tip 2502 to pass through and sharp shaft 2504 to temporarily reside. A sensor ledge 2212 can define a sensor position in a horizontal plane, prevent a sensor from lifting connector 2300 off of posts and maintain sensor 104 parallel to a plane of connector seals. It can also define sensor bend geometry and minimum bend radius. It can limit sensor travel in a vertical direction and prevent a tower from protruding above an electronics housing surface and define a sensor tail length below a patch surface. A sensor wall 2216 can constrain a sensor and define a sensor bend geometry and minimum bend radius.

FIGS. 13A and 13B are perspective views depicting an example embodiment of connector 2300 in an open state and a closed state, respectively. Connector 2300 can be made of silicone rubber that encapsulates compliant carbon impregnated polymer modules that serve as electrical conductive contacts 2302 between sensor 104 and electrical circuitry contacts for the electronics within housing 706. The connector can also serve as a moisture barrier for sensor 104 when assembled in a compressed state after transfer from a container to an applicator and after application to a user's skin. A plurality of seal surfaces 2304 can provide a watertight seal for electrical contacts and sensor contacts. One or more hinges 2208 can connect two distal and proximal portions of connector 2300.

FIG. 14 is a perspective view depicting an example embodiment of sensor 104. A neck 2406 can be a zone which allows folding of the sensor, for example ninety degrees. A membrane on tail 2408 can cover an active analyte sensing element of the sensor 104. Tail 2408 can be the portion of sensor 104 that resides under a user's skin after insertion. A flag 2404 can contain contacts and a sealing surface. A biasing tower 2412 can be a tab that biases the tail 2408 into sharp slot 2208. A bias fulcrum 2414 can be an offshoot of biasing tower 2412 that contacts an inner surface of a needle to bias a tail into a slot. A bias adjuster 2416 can reduce a localized bending of a tail connection and prevent sensor trace damage. Contacts 2418 can electrically couple the active portion of the sensor to connector 2300. A service loop 2420 can translate an electrical path from a vertical direction ninety degrees and engage with sensor ledge 2212 (FIG. 12B).

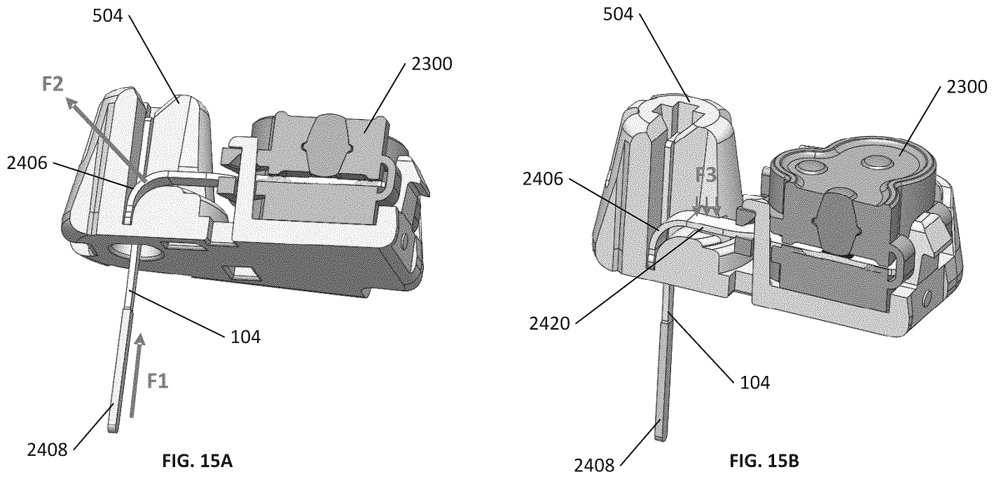

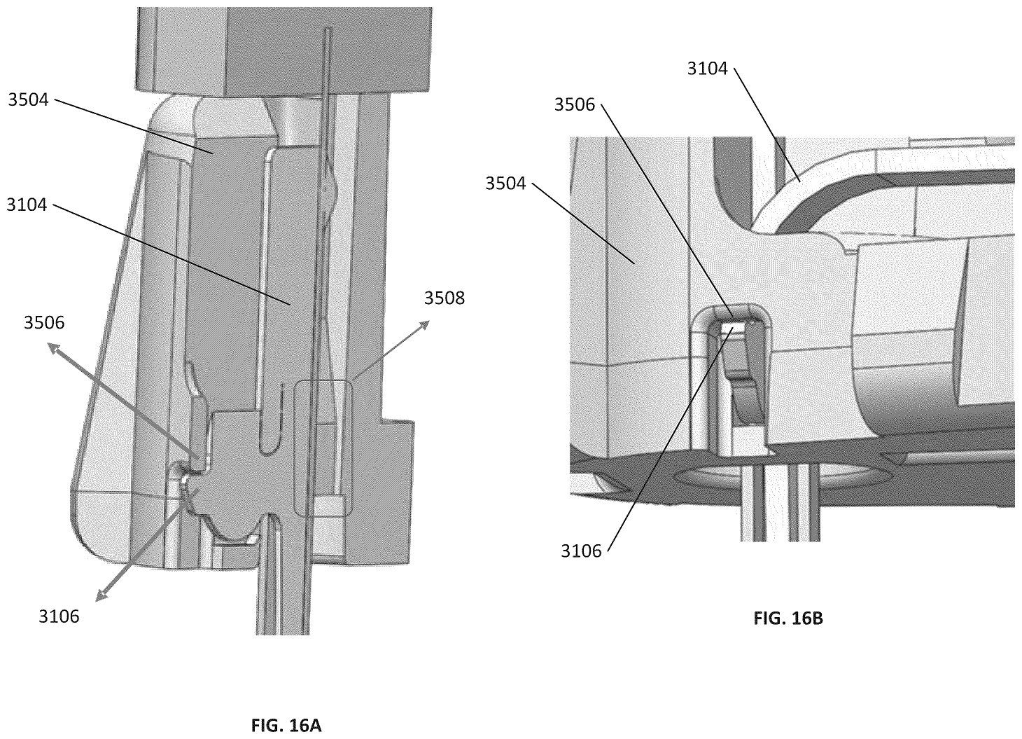

FIGS. 15A and 15B are bottom and top perspective views, respectively, depicting an example embodiment of a sensor module assembly comprising sensor module 504, connector 2300, and sensor 104. According to one aspect of the aforementioned embodiments, during or after insertion, sensor 104 can be subject to axial forces pushing up in a proximal direction against sensor 104 and into the sensor module 105, as shown by force, F1, of FIG. 15A. According to some embodiments, this can result in an adverse force, F2, being applied to neck 2406 of sensor 104 and, consequently, result in adverse forces, F3, being translated to service loop 2420 of sensor 104. In some embodiments, for example, axial forces, F1, can occur as a result of a sensor insertion mechanism in which the sensor is designed to push itself through the tissue, a sharp retraction mechanism during insertion, or due to a physiological reaction created by tissue surrounding sensor 104 (e.g., after insertion).

FIGS. 16A and 16B are close-up partial views of an example embodiment of a sensor module assembly having certain axial stiffening features. In a general sense, the embodiments described herein are directed to mitigating the effects of axial forces on the sensor as a result of insertion and/or retraction mechanisms, or from a physiological reaction to the sensor in the body. As can be seen in FIGS. 16A and 16B, according to one aspect of the embodiments, sensor 3104 comprises a proximal portion having a hook feature 3106 configured to engage a catch feature 3506 of the sensor module 3504. In some embodiments, sensor module 3504 can also include a clearance area 3508 to allow a distal portion of sensor 3104 to swing backwards during assembly to allow for the assembly of the hook feature 3106 of sensor 3104 over and into the catch feature 3506 of sensor module 3504.

According to another aspect of the embodiments, the hook and catch features 3106, 3506 operate in the following manner. Sensor 3104 includes a proximal sensor portion, coupled to sensor module 3504, as described above, and a distal sensor portion that is positioned beneath a skin surface in contact with a bodily fluid. As seen in FIGS. 16A and 16B, the proximal sensor portion includes a hook feature 3106 adjacent to the catch feature 3506 of sensor module 3504. During or after sensor insertion, one or more forces are exerted in a proximal direction along a longitudinal axis of sensor 3104. In response to the one or more forces, hook feature 3106 engages catch feature 3506 to prevent displacement of sensor 3104 in a proximal direction along the longitudinal axis.

According to another aspect of the embodiments, sensor 3104 can be assembled with sensor module 3504 in the following manner. Sensor 3104 is loaded into sensor module 3504 by displacing the proximal sensor portion in a lateral direction to bring the hook feature 3106 in proximity to the catch feature 3506 of sensor module 3504. More specifically, displacing the proximal sensor portion in a lateral direction causes the proximal sensor portion to move into clearance area 3508 of sensor module 3504.

Although FIGS. 16A and 16B depict hook feature 3106 as a part of sensor 3104, and catch feature 3506 as a part of sensor module 3504, those of skill in the art will appreciate that hook feature 3106 can instead be a part of sensor module 3504, and, likewise, catch feature 3506 can instead be a part of sensor 3106. Similarly, those of skill in the art will also recognize that other mechanisms (e.g., detent, latch, fastener, screw, etc.) implemented on sensor 3104 and sensor module 3504 to prevent axial displacement of sensor 3104 are possible and within the scope of the present disclosure.

### Example Embodiments of Sharp Modules

FIG. 17A is a perspective view depicting an example embodiment of sharp module 2500 prior to assembly within sensor module 504 (FIG. 6B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 11). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500. Further details regarding embodiments of sharp modules, sharps, their components, and variants thereof, are described in U.S. Patent Publication No. 2014/0171771, which is incorporated by reference herein in its entirety and for all purposes.

FIGS. 17B, 17C, and 17D depict example embodiments of plastic sharp modules. By way of background, according to one aspect of the embodiments, a plastic sharp can be advantageous in at least two respects.

First, relative to a metallic sharp, a plastic sharp can cause reduced trauma to tissue during the insertion process into the skin. Due to their manufacturing process, e.g., chemical etching and mechanical forming, metallic sharps are typically characterized by sharp edges and burrs that can cause trauma to tissue at the insertion site. By contrast, a plastic sharp can be designed to have rounded edges and a smooth finish to reduce trauma as the sharp is positioned through tissue. Moreover, those of skill in the art will understand that reducing trauma during the insertion process can lead to reduced ESA and improve accuracy in analyte level readings soon after insertion.

Second, a plastic sharp can simplify the applicator manufacturing and assembly process. As with earlier described embodiments, certain applicators are provided to the user in two pieces: (1) an applicator containing the sharp and sensor electronics in a sensor control unit, and (2) a sensor container. This requires the user to assemble the sensor into the sensor control unit. One reason for a two-piece assembly is to allow for electron beam sterilization of the sensor to occur separately from the applicator containing the metallic sharp and the sensor electronics. Metallic sharps, e.g., sharps made of stainless steel, have a higher density relative to sharps made of polymeric or plastic materials. As a result, electron beam scatter from an electron beam striking a metallic sharp can damage the sensor electronics of the sensor control unit. By utilizing a plastic sharp, e.g., a sharp made of polymeric materials, and additional shielding features to keep the electron beam path away from the sensor electronics, the applicator and sensor can be sterilized and packaged in a single package, thereby reducing the cost to manufacture and simplifying the assembly process for the user.

Referring to FIG. 17B, a perspective view of an example embodiment of plastic sharp module 2550 is shown, and can include a hub 2562 coupled to a proximal end of the sharp, sharp shaft 2554, a sharp distal tip 2556 configured to penetrate a skin surface, and a sensor channel 2558 configured to receive at least a portion of an analyte sensor 104. Any or all of the components of sharp module 2550 can be comprised of a plastic material such as, for example, a thermoplastic material, a liquid crystal polymer (LCP), or a similar polymeric material. According to some embodiments, for example, the sharp module can comprise a polyether ether ketone material. In other embodiments, silicone or other lubricants can be applied to an external surface of the sharp module and/or incorporated into the polymer material of the sharp module, to reduce trauma caused during the insertion process. Furthermore, to reduce trauma during insertion, one or more of sharp shaft 2554, sharp distal tip 2556, or alignment feature 2568 (described below) can include filleted and/or smoothed edges.

According to some embodiments, when assembled, the distal end of the analyte sensor can be in a proximal position relative to the sharp distal tip 2556. In other embodiments, the distal end of the analyte sensor and the sharp distal tip 2556 are co-localized.

According to another aspect of some embodiments, plastic sharp module 2550 can also include an alignment feature 2568 configured to prevent rotational movement along a vertical axis 2545 of sharp module 2550 during the insertion process, wherein the alignment feature 2568 can be positioned along a proximal portion of sharp shaft 2554.

FIGS. 17C and 17D are a side view and a perspective view, respectively, depicting another example embodiment of a plastic sharp module 2570. Like the embodiment described with respect to FIG. 17B, plastic sharp module 2570 can include a hub 2582 coupled to a proximal end of the sharp, a sharp shaft 2574, a sharp distal tip 2576 configured to penetrate a skin surface, and a sensor channel 2578 configured to receive at least a portion of an analyte sensor 104. Any or all of the components of sharp module 2570 can be comprised of a plastic material such as, for example, a thermoplastic material, LCP, or a similar polymeric material. In some embodiments, silicone or other lubricants can be applied to an external surface of sharp module 2570 and/or incorporated into the polymer material of sharp module 2570, to reduce trauma caused during the insertion process.

According to some embodiments, sharp shaft 2574 can include a distal portion 2577 that terminates at distal tip 2576, in which at least a portion of sensor channel 2578 is disposed. Sharp shaft 2574 can also have a proximal portion 2575 that is adjacent to distal portion 2577, wherein the proximal portion 2575 is solid, partially solid, or hollow, and is coupled to hub 2582. Although FIGS. 17C and 17D depict sensor channel 2578 as being located only within distal portion 2577, those of skill in the art will understand that sensor channel 2578 can also extend through a majority of, or along the entire length of, sharp shaft 2574 (e.g., as shown in FIG. 17B), including through at least a portion of proximal portion 2575. In addition, according to another aspect of some embodiments, at least a portion of proximal portion 2575 can have a wall thickness that is greater than the wall thickness of distal portion 2577, to reduce the possibility of stress buckling of the sharp during the insertion process. According to another aspect of some embodiments, plastic sharp module 2570 can include one or more ribs (not shown) adjacent to sharp hub portion 2582 to reduce the compressive load around hub 2582, and to mitigate stress buckling of the sharp during the insertion process.

FIG. 17E is a cross-sectional view depicting an example embodiment of an applicator 150 with a plastic sharp module during an electron beam sterilization process. As indicated by the rectangular area, A, an electron beam is focused on sensor 104 and plastic sharp 2550 of applicator 150 during a sterilization process. According to some embodiments, a cap 708 has been secured to applicator housing 702 to seal sensor control device 102 within applicator 150. During the sterilization process, electron beam scatter, as indicated by the diagonal arrows originating from plastic sharp 2550, in the direction and path of sensor electronics 160 has been reduced because a plastic sharp 2550 has been utilized instead of a metallic sharp. Although FIG. 17E depicts a focused electron beam sterilization process, those of skill in the art will recognize that an applicator with a plastic sharp module embodiment can also be utilized during a non-focused electron beam sterilization process.

FIG. 17F is a flow diagram depicting an example embodiment method 1100 for sterilizing an applicator assembly, according to the embodiments described above. At Step 1105, a sensor control device 102 is loaded into the applicator 150. Sensor control device 102 can include various components, including an electronics housing, a printed circuit board positioned within the electronics housing and containing processing circuitry, an analyte sensor extending from a bottom of the electronics housing, and a plastic sharp module having a plastic sharp that extends through the electronics housing. According to some embodiments, the plastic sharp can also receive the portion of the analyte sensor extending from the bottom of the electronics housing. As previously described, at Step 1110, a cap 708 is secured to the applicator housing 702 of applicator 150, thereby sealing the sensor control device 102 within applicator 150. At Step 1115, the analyte sensor 104 and plastic sharp 2550 are sterilized with radiation while sensor control device 102 is positioned within applicator 150.

According to some embodiments, sensor control device 102 can also include at least one shield positioned within the electronics housing, wherein the one or more shields are configured to shield the processing circuitry from radiation during the sterilization process. In some embodiments, the shield can comprise a magnet that generates a static magnetic field to divert radiation away from the processing circuitry. In this manner, the combination of the plastic sharp module and the magnetic shields/deflectors can operate in concert to protect the sensor electronics from radiation during the sterilization process.

Another example embodiment of a sharp designed to reduce trauma during a sensor insertion and retraction process will now be described. More specifically, certain embodiments described herein are directed to sharps comprising a metallic material (e.g., stainless steel) and manufactured through a coining process. According to one aspect of the embodiments, a coined sharp can be characterized as having a sharp tip with all other edges comprising rounded edges. As previously described, metallic sharps manufactured through a chemical etching and mechanical forming process can result in sharp edges and unintended hook features. For example, FIG. 17G is a photograph depicting a metallic sharp 2502 manufactured by a chemical etching and mechanical forming process. As can be seen in FIG. 17G, metallic sharp 2502 includes a sharp distal tip 2506 with a hook feature. These and other unintended transition features can result in increased trauma to tissue during a sensor insertion and retraction process. By contrast, FIG. 17H is a photograph depicting a coined sharp 2602, that is, a metallic sharp manufactured through a coining process. As can be seen in FIG. 17H, coined sharp 2602 also includes a sharp distal tip 2606. Coined sharp 2602, however, includes only smooth, rounded edges without any unintended sharp edges or transitions.

As with previously described sharp embodiments, the coined sharp 2602 embodiments described herein can also be assembled into a sharp module having a sharp portion and a hub portion. Likewise, the sharp portion comprises a sharp shaft, a sharp proximal end coupled to a distal end of the hub portion, and a sharp distal tip configured to penetrate a skin surface. According to one aspect of the embodiments, one or all of the sharp portion, the sharp shaft, and/or the sharp distal tip of a coined sharp 2602 can comprise one or more rounded edges.

Furthermore, it will be understood by those of skill in the art that the coined sharp 2602 embodiments described herein can similarly be used with any of the sensors described herein, including in vivo analyte sensors that are configured to measure an analyte level in a bodily fluid of a subject. For example, in some embodiments, coined sharp 2602 can include a sensor channel (not shown) configured to receive at least a portion of an analyte sensor. Likewise, in some embodiments of the sharp module assembly utilizing a coined sharp 2602, the distal end of the analyte sensor can be in a proximal position relative to the sharp distal tip 2606. In other embodiments, the distal end of the analyte sensor and the sharp distal tip 2606 are co-localized.

Other example embodiments of sharps designed to reduce trauma during a sensor insertion process will now be described. Referring back to FIG. 17A, an example embodiment of sharp module 2500 (shown without analyte sensor) is depicted, and includes a sharp 2502 comprising a sensor channel having a U-shaped geometry configured to receive at least a portion of an analyte sensor, and a distal tip 2506 configured to penetrate a skin surface during the sensor insertion process.

In certain embodiments, sharp module can include a sharp having a distal tip with an offset geometry configured to create a smaller opening in the skin relative to other sharps (e.g., sharp 2502 depicted in FIG. 17A). Turning to FIG. 17I, a perspective view of an example embodiment of a sharp module 2620 (with analyte sensor 104) having an offset tip portion is shown. Similar to the previously described sharp modules, sharp module 2620 can include a sharp shaft 2624 coupled to hub 2632 at a proximal end, sensor channel 2628 configured to receive at least a portion of analyte sensor 104, and a distal tip 2626 configured to penetrate a skin surface during the sensor insertion process.

According to one aspect of the embodiment, one or more sidewalls 2629 that form sensor channel 2628 are disposed along sharp shaft 2624 at a predetermined distance, Dsc, from distal tip 2626. In certain embodiments, predetermined distance, Dsc, can be between 1 mm and 8 mm. In other embodiments, predetermined distance, Dsc, can be between 2 mm and 5 mm. Those of skill in the art will recognize that other predetermined distances, Dsc, can be utilized and are fully within the scope of the present disclosure. In other words, according to some embodiments, sensor channel 2628 is in a spaced relation to distal tip 2626. In this regard, distal tip 2626 has a reduced cross-sectional footprint relative to, for example, distal tip 2506 of sharp module 2500, whose sensor channel is adjacent to distal tip 2506. According to another aspect of the embodiment, at the terminus of distal tip 2626 is an offset tip portion 2627 configured to prevent sensor tip 2408 from being damaged during insertion and to create a small opening in the skin. In some embodiments, offset tip portion 2627 can be a separate element coupled to a distal end of sharp shaft 2624. In other embodiments, offset tip portion 2627 can be formed from a portion of distal tip 2506 or sharp shaft 2624. During insertion, as the sharp moves into the skin surface, offset tip portion 2627 can cause the skin surrounding the skin opening to stretch and widen in a lateral direction without further cutting of skin tissue. In this regard, less trauma results during the sensor insertion process.

Referring next to FIG. 17J, a perspective view of another example embodiment of a sharp module 2640 (with analyte sensor 104) having an offset tip portion is shown. Like the previous embodiments, sharp module 2640 can include a sharp shaft 2644 coupled to hub 2652 at a proximal end, sensor channel 2648 configured to receive at least a portion of analyte sensor 104, and a distal tip 2646 configured to penetrate a skin surface during the sensor insertion process. According to one aspect of the embodiment, sensor channel 2648 can comprise a first sidewall 2649a and a second sidewall 2649b, wherein first sidewall 2649a extends to the distal tip 2646, wherein a terminus of first sidewall 2649a forms the offset tip portion 2647, and wherein second sidewall 2649b is disposed along sharp shaft 2644 at a predetermined distance from distal tip 2646, and wherein a terminus of second sidewall 2649b is proximal to the terminus of first sidewall 2649a. Those of skill in the art will appreciate that in other embodiments, second sidewall 2649b can extend to the distal tip 2646 to form the offset tip portion 2647, instead of first sidewall 2649a. In addition, offset tip portion 2647 can be formed from a third or fourth sidewall (not shown), and such geometries are fully within the scope of the present disclosure.

With respect to the sharp and sharp module embodiments described herein, those of skill in the art will recognize that any or all of the components can comprise either a metallic material, such as stainless steel, or a plastic material, such as a liquid crystal polymer. Furthermore, it will be understood by those of skill in the art that any of the sharp and/or sharp module embodiments described herein can be used or combined with any of the sensors, sensor modules, sensor carriers, sheaths, applicator devices, or any of the other analyte monitoring system components described herein.

### Example Embodiments of Reusable Powered Applicator

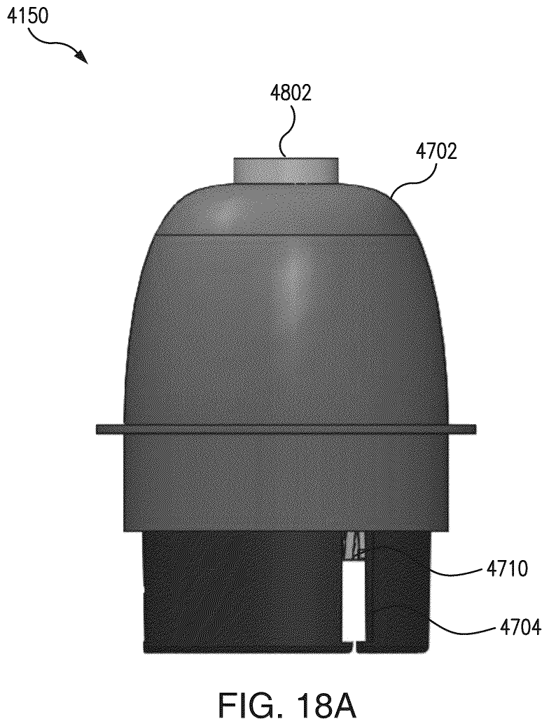

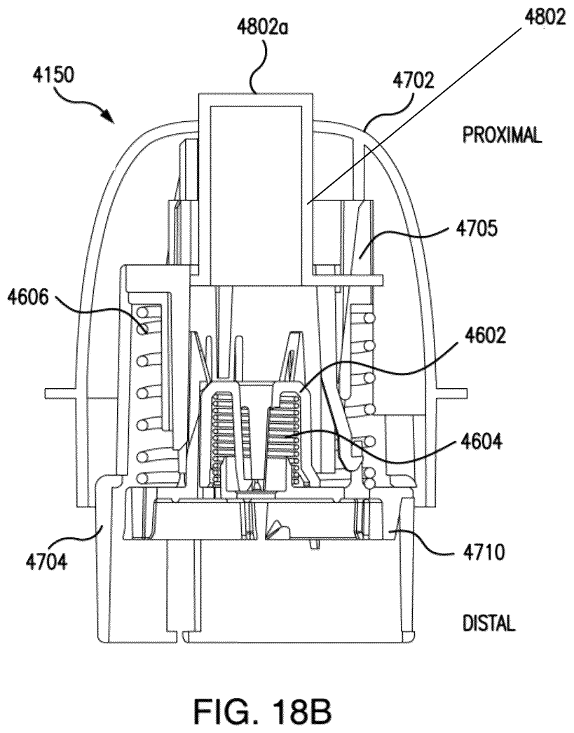

FIGS. 18A and 18B are a front view and a cross-sectional view, respectively, depicting an example embodiment of a reusable powered applicator 4150 for insertion of an analyte sensor in a subject. According to one aspect of the embodiments, actuator 4802 of powered applicator 4150 operates as a trigger that releases under light pressure and activates a drive spring 4606 to push sensor carrier 4710 downward and insert a sharp and the analyte sensor in the subject. After insertion of the analyte sensor, a retraction spring 4604 causes the sharp to withdraw from the subject. According to an aspect of the embodiments, after delivery of a first analyte sensor, powered applicator 4150 can be reloaded and reused for subsequent delivery of another analyte sensor in the subject. For example, used sharp (not shown) can be removed, retraction spring 4604 and drive spring 4606 can be reloaded, and actuator 4802 reset so that powered applicator 4150 can be reused, as described in further detail below. According to an aspect of the embodiments, powered applicator 4150 can provide for a higher, more controlled insertion speed relative to an applicator that relies upon manual force for insertion. Powered applicator 4150 is further advantageous in that it can improve upon insertion success and can also reduce trauma at the insertion site, relative to an applicator that relies upon manual force for insertion. Furthermore, powered applicator 4150 can be advantageous in that it can be reused thereby reducing overall cost and environmental impact.

Referring to FIGS. 18A and 18B, the various components of powered applicator 4150 will now be described. As can be seen in in FIG. 18A as a front view and FIG. 18B as a cross-sectional view of an assembled powered applicator 4150 (in an initial state), powered applicator 4150 can include the following components: housing 4702, actuator 4802, sharp carrier 4602, retraction spring 4604, sheath 4704, firing pin 4705, drive spring 4606, sensor carrier 4710. Furthermore, although not depicted, powered applicator 4150 can also include any of the embodiments of sensor control units, analyte sensors, and sharps described herein, or in other publications which have been incorporated by reference.

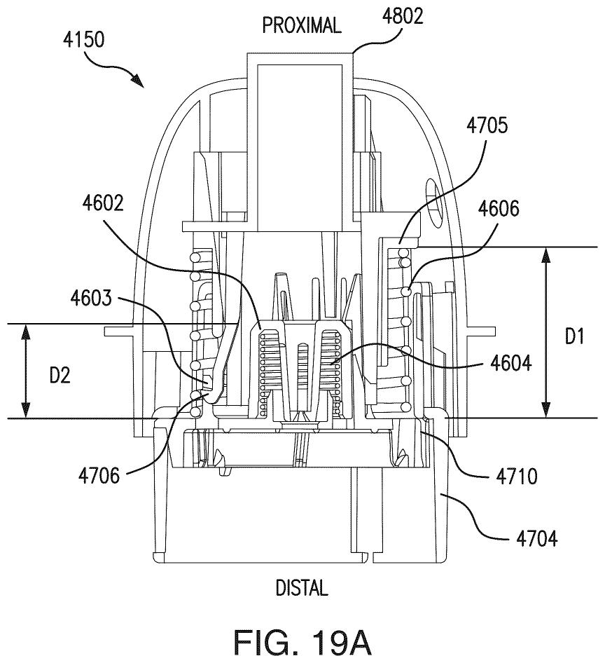

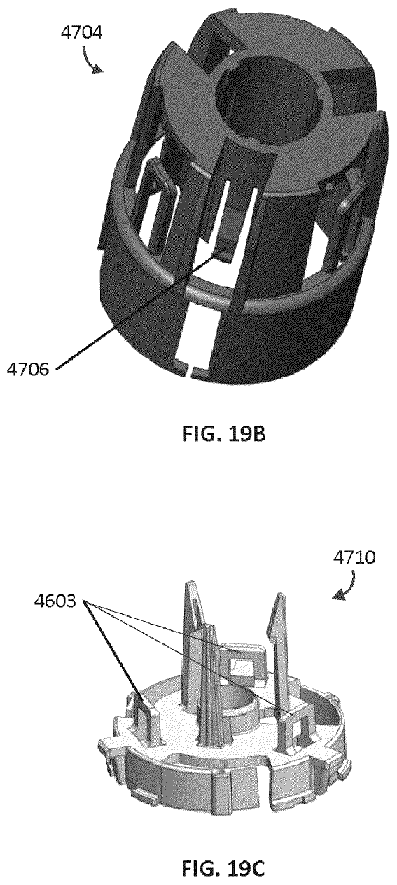

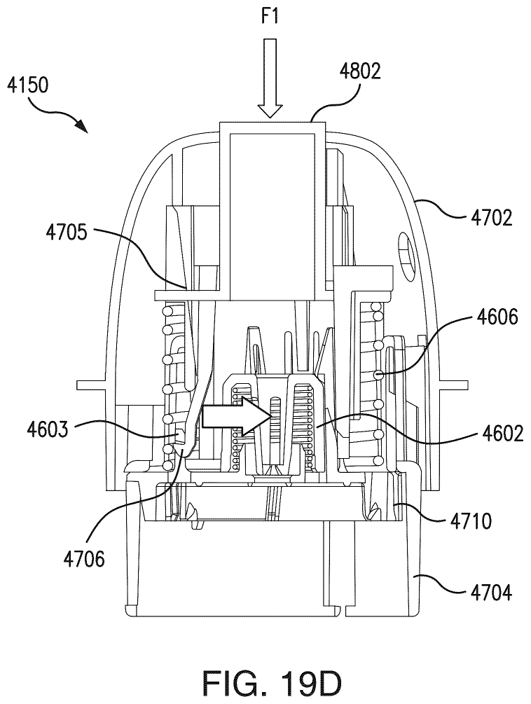

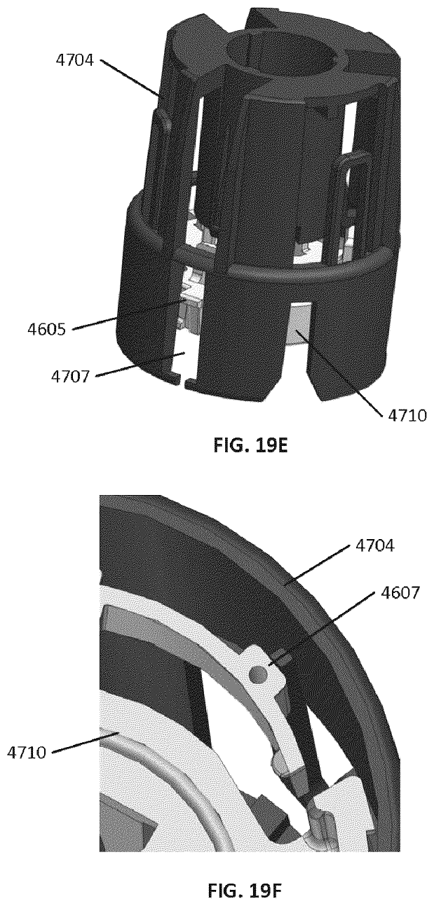

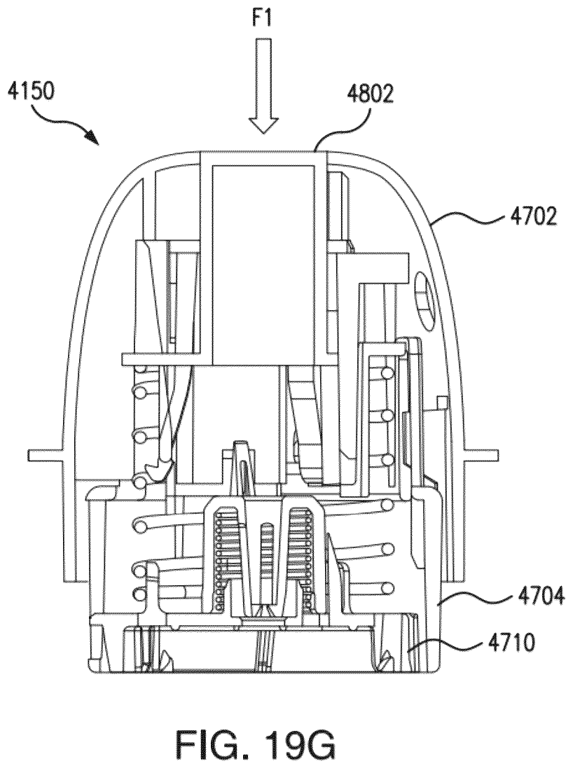

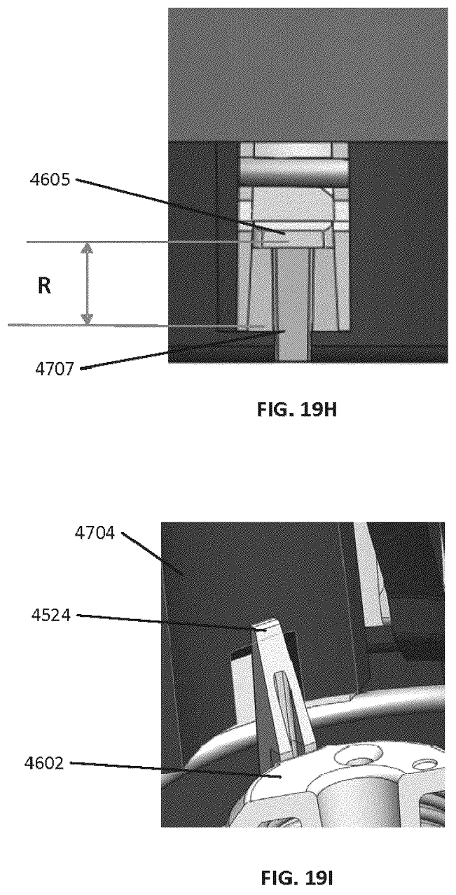

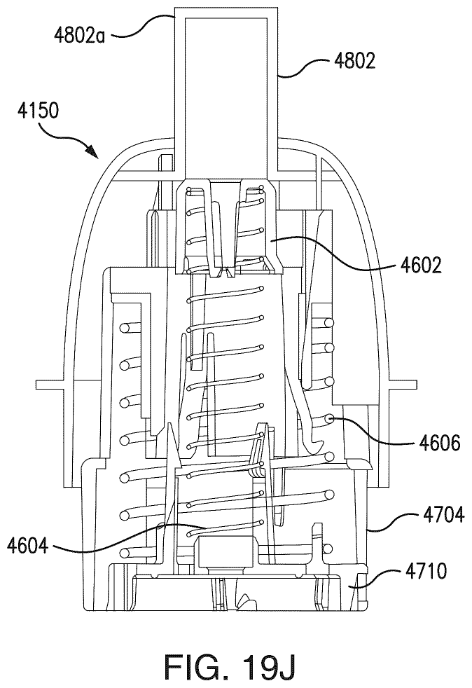

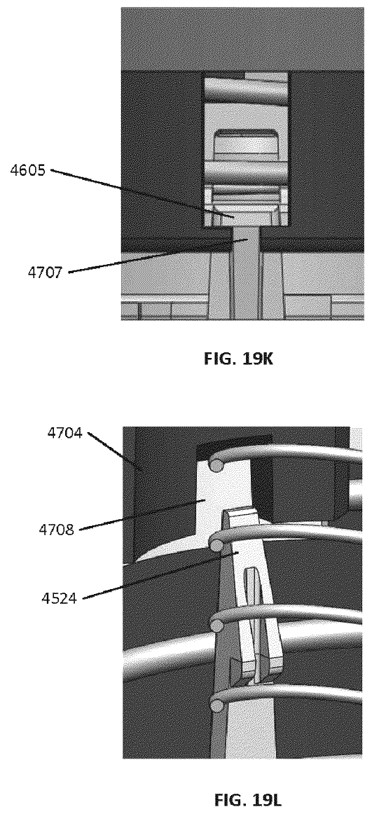

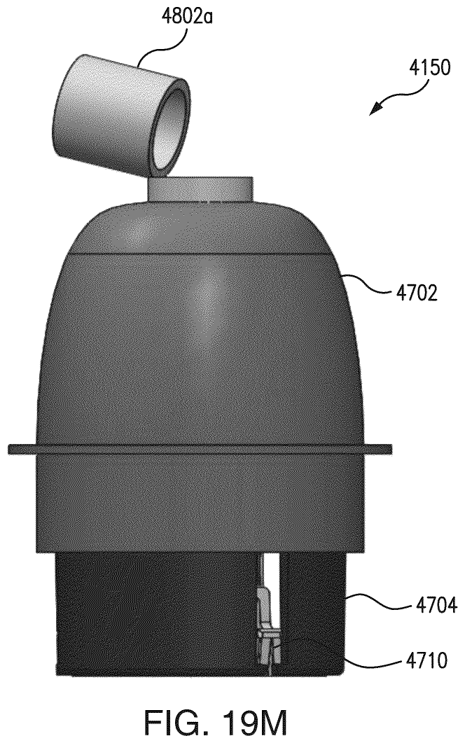

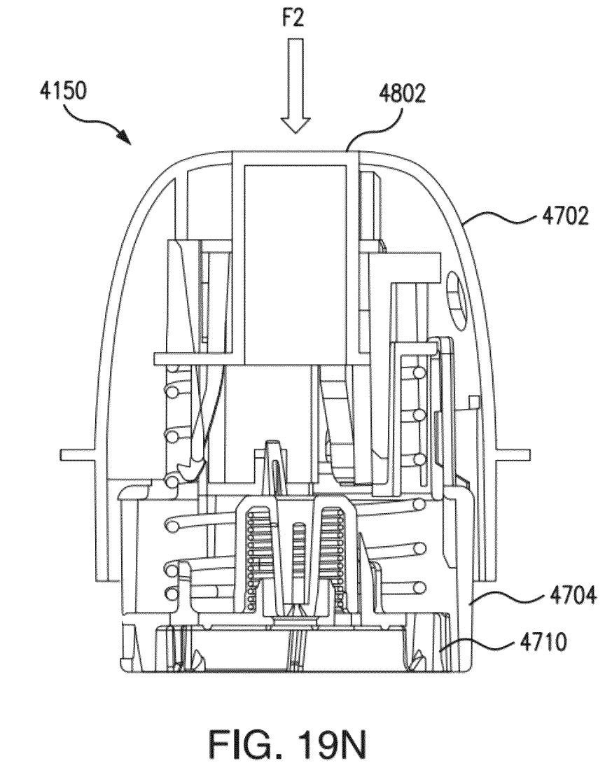

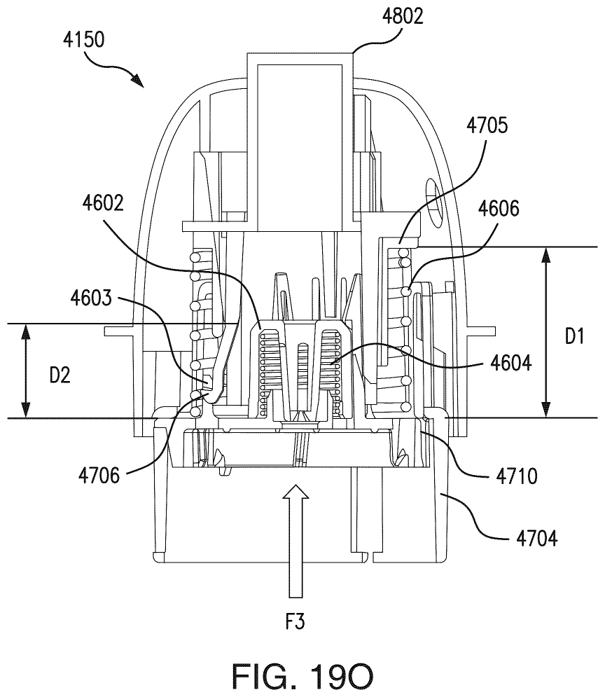

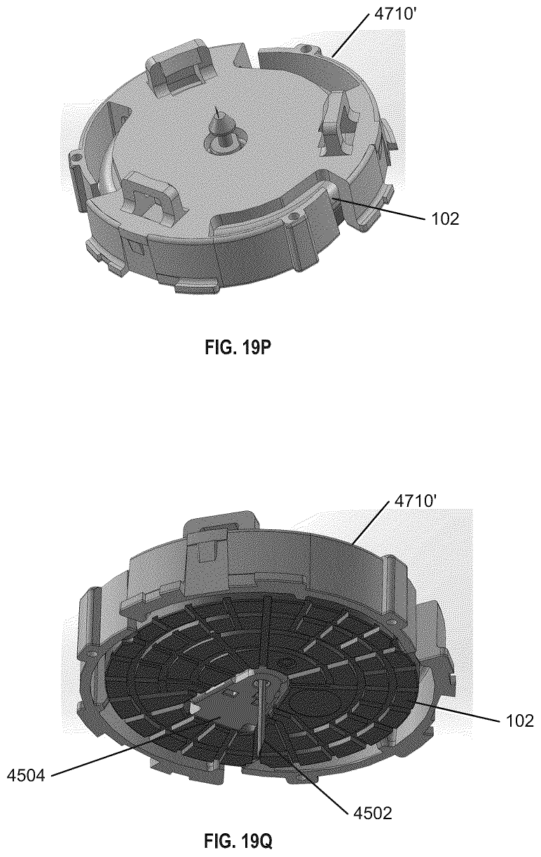

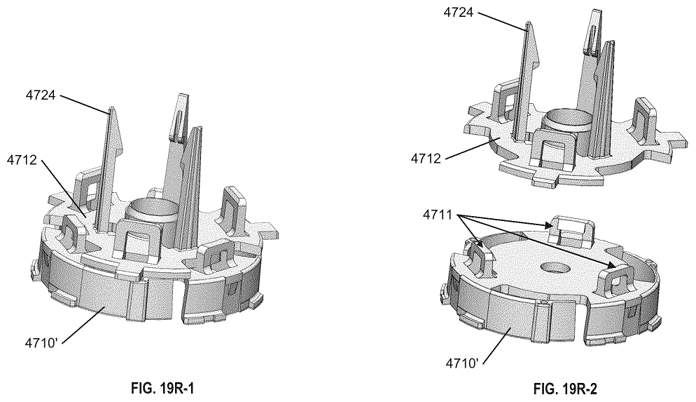

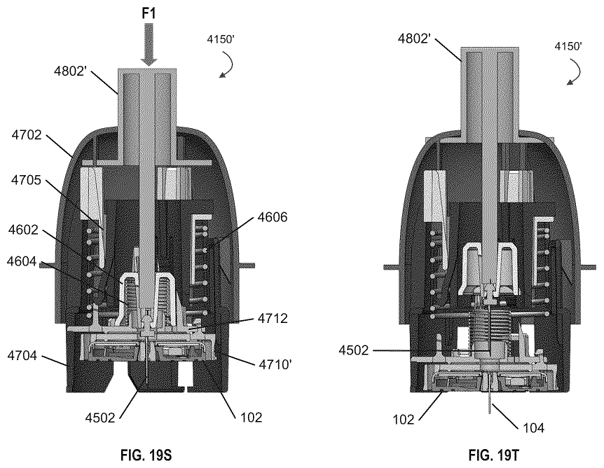

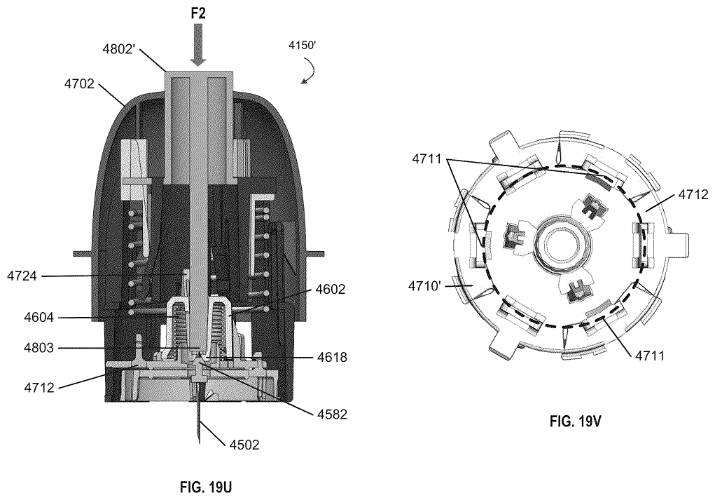

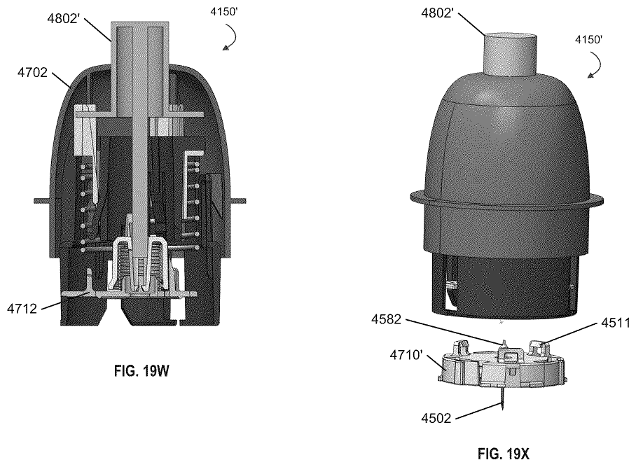

FIGS. 19A to 190 are various views depicting an example embodiment of a reusable powered applicator 4150 during various stages of deployment and rearmament.

FIG. 19A is a cross-sectional view showing powered applicator 4150 in an initial state, wherein a distal end of applicator 4150 is ready to be positioned on a subject's skin surface. In the initial state, the drive spring 4606 and retraction spring 4604 are each in a preloaded state. Drive spring 4606 includes a first end coupled to firing pin 4705 and a second end coupled to sensor carrier 4710. Retraction spring 4604 includes a first end coupled to sharp carrier 4602 and a second end coupled to sensor carrier 4710. As best seen in FIG. 19A, in the initial state, sensor carrier 4710 and sharp carrier 4602 are in a first position within applicator 4150, in a spaced relation with the skin surface. Further, as best seen in FIG. 19A, in the initial state, actuator 4802 is in an initial, ready-to-fire position within powered applicator 4150. The ready-to-fire position of the actuator can be a first position with the sensor carrier and the sharp carrier in the proximal portion of the reusable applicator.

According to an aspect of the embodiments, in the initial state, sensor carrier 4710 can be coupled to sheath 4704 by one or more latch-tab structures. FIG. 19B depicts a perspective view of sheath 4704 comprising one or more sheath tabs 4706. FIG. 19C depicts a perspective view of sensor carrier 4710 comprising one or more corresponding sensor carrier latches 4603. In the initial state, each of the one or more sensor carrier latches 4603 is engaged to a corresponding sheath latch 4706, as best seen in FIG. 19A. Although FIGS. 19B and 19C depict three sheath tabs 4706 and three sensor carrier latches 4603, those of skill in the art will appreciate that fewer or more latch-tab structures can be utilized, and those embodiments are fully within the scope of the present disclosure.

FIG. 19D is a cross-sectional view showing powered applicator 4150 in a firing state, wherein a force, F1, is applied to actuator 4802 in a distal direction (as indicated by the arrow). According to one aspect of the embodiments, application of force, F1, causes actuator 4802 to move in a distal direction, thereby causing firing pin 4705 to move along sheath 4704 in a distal direction and, subsequently, disengages sheath tabs 4706 from sensor carrier latches 4603 (as indicated by the white arrow). Disengagement of sheath tabs 4706 from sensor carrier latches 4603 causes drive spring 4606 to actuate or expand in a distal direction, thereby “firing” applicator 4150. As drive spring 4606 expands in a distal direction, sensor carrier 4710 and sharp carrier 4602 are displaced, also in a distal direction, to a second position adjacent to the skin surface.

According to some embodiments, prior to disengagement of sheath tabs 4706, application of force, F1, can increase the load on drive spring 4606 by further compressing it.

According to one aspect of the embodiments, the “cylinder-on-cylinder” design of sheath 4704 and firing pin 4705 can provide for a stable and simultaneous release of all three sensor carrier latches 4603. Furthermore, in some embodiments, certain features can provide for enhanced stability while sensor carrier 4710 and sharp carrier 4602 are being displaced from the first position to the second position. For example, as seen in FIG. 19E, sensor carrier 4710 can include one or more sensor carrier tabs 4605 that are configured to travel in a distal direction along one or more sheath rails 4707 of the sheath 4704. In addition, as seen in FIG. 19F, according to some embodiments, sensor carrier 4710 can include one or more sensor carrier bumpers 4607, each of which can be biased against an internal surface of sheath 4704 while the sensor carrier 4710 and sharp carrier 4602 are displaced from the first position to the second position.

FIG. 19G is a cross-sectional view showing powered applicator 4150 in an insertion state, during which the sharp and a portion of the analyte sensor (not shown) are positioned under the skin surface and in contact with a bodily fluid of the subject. Moreover, at this stage, a sharp retraction process has not yet been initiated. Additionally, as best seen in FIG. 19G, after powered applicator 4150 has been “fired,” i.e., in the insertion state, actuator 4802 may be in a second position, e.g., flush against housing 4702, with the sensor carrier 4710 and the sharp carrier 4602 in the distal portion of the reusable applicator for delivery of the first analyte sensor. While shown flush against housing, the second position of the actuator 4802 can be any depressed position relative the first position. In the second position, as best seen in FIG. 19D or 19G, the relative length of actuator 4802 to housing 4702 is less than the relative length of actuator 4802 to housing 4702 in the first position. Moreover, actuator 4802 may be biased, using, for example, a spring housed within housing 4702, such that when force F1 is removed, actuator 4802 can return to the initial position. As best seen in FIG. 19I, sensor carrier locks arms 4524 continue to be constrained by sheath 4704, thereby preventing the sharp carrier 4602 (and also the sharp) from retracting.

According to another aspect of the embodiments, during the insertion state, as sensor carrier 4710 reaches the second position, the sensor carrier 4710 and a distal portion of a sensor control unit (not shown) coupled with the sensor carrier 4710 comes into resting contact with the skin surface. According to aspects of the embodiments, sheath 4704 can include latch to hold sensor carrier 4710 in the second position. In some embodiments, the distal portion of the sensor control unit can be an adhesive surface.

Furthermore, according to some embodiments, as best seen in FIG. 19H, during the insertion state, sensor carrier tabs 4605, which are positioned within sheath rails 4707, have traveled in a distal direction to the second position, but are still positioned above a bottom portion of applicator 4150, as indicated by distance, R.

FIG. 19J is a cross-sectional view showing powered applicator 4150 in a sharp retraction state. According to one aspect of the embodiments, after the insertion state is complete, each of the sensor carrier lock arms 4524 is positioned into a sheath notch 4708, as best seen in FIG. 19L. Consequently, sensor carrier lock arms 4524, which are biased in a radially outward direction, can expand in a radially outward direction through sheath notches 4708. In turn, sensor carrier lock arms 4524 disengage from and release sharp carrier 4602, and retraction spring 4604 is free to expand or actuate in a proximal direction. As retraction spring 4604 expands in a proximal direction, sharp carrier 4602 is displaced to the third position within applicator 4150 (e.g., top of sheath 4704), which causes the sharp to withdraw from the skin surface. Notably, in the third position, because sensor carrier 4710 was latched to sheath 4704, sensor carrier 4710 remains locked in the second, distal position. Moreover, as sharp carrier 4602 is displaced proximally within powered applicator 4150, sharp carrier 4602 causes actuator 4802 to extend to a third position. As best seen in FIG. 19J, actuator 4802 in the third position is fully extended in a proximal direction past its initial position, providing visual indication to a subject that powered applicator 4150 has been used. As such, the relative length of actuator 4802 to housing 4702 in the third position is greater than the relative length of actuator 4802 to housing 4702 in the first position.

According to another aspect of the embodiments, as shown in FIG. 19M, after delivery of a first sensor, sharp can be removed from the powered applicator 4150. For example, actuator 4802 can include button or cap 4802a which may be used to access sharp carrier (as seen in FIG. 19J) and sharp hub (e.g., sharp hub 2562 as seen in FIG. 17B). In turn, subject can open button or cap 4802a to remove sharp hub (e.g., sharp hub 2562 as seen in FIG. 17B) from sharp carrier 4602 (as seen in FIG. 19J) for safe disposal. For example, sharp hub (e.g., sharp hub 2562 as seen in FIG. 19J) can be disengaged from sharp carrier 4602 (as seen in FIG. 19J) by rotating or twisting sharp hub relative to sharp carrier 4602 (as seen in FIG. 19J). For example, sharp hub can be rotated or twisted 15 degrees, 30 degrees, 45 degrees, 60 degrees, 75 degrees, 90 degrees, etc. or any degree within a range of 0-90 degrees.

FIG. 19N is a cross-sectional view showing powered applicator 4150 in an initial rearmament state, wherein a force, F2, is applied to advance actuator 4802 in a distal direction (as indicated by the arrow). According to one aspect of the embodiments, application of force, F2, causes sharp carrier 4602 to move in a distal direction towards sensor carrier 4710 until sensor carrier lock arms 4524 reengage sharp carrier 4602. Consequently, sharp carrier 4602 recompresses and reloads retraction spring 4604 for subsequent use. As best seen in FIG. 19N, in the initial rearmament state, actuator 4802 may be in a fourth position, e.g., flush against housing 4702. Moreover, actuator 4802 can be latched or locked within housing 4702 such that when force F2 is removed, actuator 4802 remains flush against housing 4702.

FIG. 19O is a cross-sectional view showing powered applicator 4150 in a final rearmament state, wherein a force, F3, is applied to sensor carrier 4710 to advance sensor carrier 4710 and sharp carrier 4602 in a proximal direction (as indicated by the arrow). Displacement of sensor carrier 4710 and sharp carrier 4602 in the proximal direction causes firing pin 4705 to move along sheath 4704 in a proximal direction and causes drive spring 4606 to compress. Subsequently, sheath tabs 4706 reengage sensor carrier latches 4603 causing drive spring 4606 to fully compress, thereby rearming powered applicator 4150. As discussed previously, the “cylinder-on-cylinder” design of sheath 4704 and firing pin 4705 can provide for a stable and simultaneous reengagement of all three sensor carrier latches 4603. As such, in the “rearmed” state, the drive spring 4606 and retraction spring 4604 are each in the preloaded state. Moreover, in the “rearmed” state, sensor carrier 4710 and sharp carrier 4602 have returned to the first position within powered applicator 4150, in a spaced relation with the skin surface. Further, as best seen in FIG. 19O, actuator 4802 is in the initial, ready-to-fire position within powered applicator 4150.

According to an aspect of the embodiments, actuator 4802 and/or powered applicator 4150 can include visual indicators corresponding to the position of actuator 4802. More specifically, actuator 4802 and/or powered applicator 4150 can include visual indicators to indicate whether actuator 4802 is in an initial, second, third, or fourth position. For example, visual indicators can include color coding along outer surface 4802a of actuator 4802, with each position being represented by a different color (e.g., green for initial position, yellow for second position, green for third position, and green for forth position), distance marking and/or words on outer surface 4802a corresponding to the different positions, etc. As a result, during use, a user can quickly ascertain the position of actuator 4802.

FIGS. 19P to 19X depict another example embodiment of a reusable powered applicator 4150′ for insertion of an analyte sensor in a subject. According to one aspect of the embodiments, reusable powered applicator 4150′ can comprise a disposable portion comprising a disposable sensor carrier 4710′, as shown in a top-down and a bottom-up perspective, respectively, in FIGS. 19P and 19Q. According to another aspect of the embodiments, the disposable sensor carrier 4710′ can be configured to releasably retain a sensor control device 102 having a sharp 4502 and sensor module 4504 disposed therethrough. Although not shown in FIGS. 19P and 19Q, the disposable portion can also include one or more of an adhesive patch configured to be adhered to the user's skin, an adhesive liner, and/or a sharp/sensor guard.

According to another aspect of the embodiments, reusable powered applicator 4150′ can further comprise a reusable portion that includes reusable applicator base 4712. FIGS. 19R-1 and 19R-2 depict perspective views of reusable applicator base 4712 and disposable sensor carrier 4710′ in coupled and uncoupled states, respectively. According to some embodiments, disposable sensor carrier 4710′ can further comprise one or more snaps or latches 4711 for coupling with a corresponding ledge of reusable applicator base 4712. In addition, reusable applicator base 4712 can comprise one or more carrier lock arms 4724 for engaging with the sharp carrier.

FIGS. 19S, 19T, 19U, and 19W are cross-sectional views depicting an example embodiment of a reusable powered applicator 4150′ in various stages of operation. FIG. 19S, for example, is a cross-sectional view depicting a reusable powered applicator 4150′ in a ready-to-fire state, in which the distal end of the reusable powered applicator 4150′ is ready to be positioned on a subject's skin surface. In the ready-to-fire state, drive spring 4606 and retraction spring 4604 are each in a preloaded state. According to an aspect of the embodiments, drive spring 4606 can include a first end coupled with the firing pin 4705, and a second end coupled to the reusable applicator base 4712. As shown in FIG. 19S, in the ready-to-fire state, disposable sensor carrier 4710′, reusable applicator base 4712, and sharp carrier 4602 are in a first position, within reusable powered applicator 4150′, in a spaced relation with the skin surface. Also, as shown in FIG. 19S, actuator 4802′ is in a ready-to-fire position, wherein a proximal portion of actuator 4802′ is at a predetermined height relative to housing 4702. Further, in the ready-to-fire state, disposable sensor carrier 4710′ is configured to retain sensor control device 102, with sharp 4502 extending therethrough.

Reusable powered applicator 4150′ is “fired” when a force, F1, is applied to actuator 4802′ in a distal direction (as indicated by the arrow). According to one aspect of the embodiments, the application of force, F1, causes actuator 4802′ to move in a distal direction, thereby causing firing pin 4705 to move along sheath 4704 in a distal direction. As firing pin 4705 advances in the distal direction, it disengages sheath tabs 4706 from sensor carrier latches (as described, e.g., with respect to FIG. 19D), which allows drive spring 4606 to expand in a distal direction. As drive spring 4606 expands in the distal direction, disposable sensor carrier 4710′, reusable applicator base 4712, sharp carrier 4602, and sensor control device 102 are also displaced in a distal direction to a second position adjacent to the skin surface (FIG. 19T).

According to some embodiments, prior to disengagement of sheath tabs 4706, application of force, F1, can increase a load on drive spring 4606 by further compressing it. As described earlier, the “cylinder-on-cylinder” design of sheath 4704 and firing pin 4705 can provide for a stable and simultaneous release of the sensor carrier latches. Furthermore, in some embodiments, certain features can provide for enhanced stability while disposable sensor carrier 4710′, reusable applicator base 4712, and sharp carrier 4602 are being displaced from the first position to the second position. For example, in some embodiments, disposable sensor carrier 4710′ can include one or more sensor carrier tabs configured to travel in a distal direction along one or more sheath rails of sheath 4704, similar to the structures described with respect to FIG. 19E. Likewise, according to some embodiments, disposable sensor carrier 4710′ can include one or more sensor carrier bumpers, each of which can be biased against an internal surface of sheath 4704 while the disposable sensor carrier 4710′, reusable applicator base 4712, and sharp carrier 4602 are displaced from the first position to the second position.

FIG. 19T is another cross-sectional view depicting reusable powered applicator 4150′ in a state after it has been fired and sensor control device 102 has been deployed. As shown in FIG. 19T, actuator 4802′ is in a returned position of increased height, which can provide a visual cue to the user that sensor control device 102 has been applied and at least a portion of sensor 104 has been successfully inserted. In some embodiments, the returned position of the actuator 4802′ can be a greater height than the position of actuator 4802′ prior to firing. In other embodiments, the returned position of the actuator 4802′ can be either the same or less than the position of actuator 4802′ prior to firing. As further shown in FIG. 19T, sharp 4502 has been automatically retracted from the skin by a retraction mechanism. In several respects, the retraction mechanism of reusable powered applicator 4150′ operates in a manner similar to the retraction mechanism of applicator 4150, as previously described with respect to FIG. 19J. Moreover, according to another aspect of the embodiments, the retraction mechanism of reusable powered applicator 4150′ can be configured to output an audible cue to indicate a successful insertion. Subsequently, reusable powered applicator 4150′ can be removed from the insertion site on the skin, leaving sensor control device 102 deployed on the skin with at least a portion of sensor 104 inserted.

FIG. 19U is another cross-sectional view depicting reusable powered applicator 4150′ during a sharp ejection stage. According to one aspect of the embodiments, after reusable powered applicator 4150′ has been removed from the skin, a force, F2, can then be applied to actuator 4802′ to cause ejection of sharp 4502. As shown in FIG. 19U, application of force, F2, can cause a proximal portion of actuator 4802′ to become substantially or fully depressed relative to housing 4702 of reusable powered applicator 4150′. According to another aspect of the embodiments, application of force, F2, can further cause the actuator's distal portion 4803 to advance in a distal direction until distal portion 4803 contacts sharp carrier retention arms 4618 of sharp carrier 4602. Advancement of distal portion 4803, from application of force, F2, further causes the sharp carrier retention arms 4618 to spread apart and disengage from sharp hub 4582 on the proximal end of sharp 4502. Subsequently, sharp 4502, which is no longer retained by sharp carrier retention arms 4618, can be ejected by distal portion 4803 as it continues to advance in a distal direction. In some embodiments, for example, after being removed from the skin surface, reusable powered applicator 4150′ can be positioned over a sharp container, such that sharp 4502 is safely ejected from the reusable powered applicator 4150′ into the sharp container.

According to another aspect of the embodiments, a sensor carrier ejection stage can occur after, or concurrently with, the sharp ejection stage. FIG. 19V is a top down view of reusable applicator base 4712 and disposable sensor carrier 4710′ in a coupled state (also shown in FIG. 19R-1). Disposable sensor carrier 4710′ can be configured to disengage from reusable applicator base 4712 in response to an application of force on the one or more snaps or latches 4711 of disposable sensor carrier 4710′. In some embodiments, for example, the application of force, F2, to actuator 4802′ (as shown in FIG. 19U) can further cause a cylindrical base portion of sharp carrier 4602 (e.g., as reflected by the dashed circle) to push down on the one or more snaps or latches 4711 of disposable sensor carrier 4710′. In other embodiments, one or more distally extending features of actuator 4802′ (not shown) can each be configured to interface with a corresponding snap or latch 4711 of disposable sensor carrier 4710′. In response to a downward force applied to the one or more snaps or latches 4711, sensor carrier 4710′ is disengaged from reusable applicator base 4712 and ejected from the reusable powered applicator 4150′.

Referring again to FIG. 19U, according to another aspect of the embodiments, application of force, F2, can further cause sharp carrier 4602 to advance in a distal direction until carrier lock arms 4724 of reusable applicator base 4712 reengage sharp carrier 4602. Consequently, sharp carrier 4602 recompresses and reloads retraction spring 4604 for subsequent use. The reloading of retraction spring 4604 can occur during either or both of the sharp ejection stage or the sensor carrier ejection stage.

FIG. 19W is another cross-sectional view depicting reusable powered applicator 4150′ in a ready-to-load state. According to one aspect of the embodiments, in the ready-to-load state, the sharp and disposable sensor carrier have been ejected, the sharp carrier has been re-latched, the retraction spring has been reloaded, and the reusable powered applicator 4150′ is ready to accept a new disposable sensor carrier, along with a new sensor control device and a new sharp. According to some embodiments, the position of the actuator 4802′ can serve as a visual indicator of the ready-to-load state. For example, in some embodiments, in the ready-to-load state, the actuator 4802′ can be at a height relative to housing 4702 that is less than either of the height of actuator 4802′ in the ready-to-fire state (FIG. 19S) or the height of actuator 4802′ in the deployed state (FIG. 19T).

FIG. 19X is a perspective view depicting reusable powered applicator 4150′ in a ready-to-load state. As further shown in FIG. 19X, a new disposable assembly comprising a new disposable sensor carrier 4710′ and a new sharp 4502 is positioned below the reusable powered applicator 4150′. In some embodiments, disposable assembly can be preloaded with a new sensor control device 102 (not shown). In other embodiments, the new sensor control device 102 (now shown) can be loaded into the disposable assembly after it is inserted into reusable powered applicator 4150′. According to another aspect of the embodiments, the new disposable assembly is received into a distal end of reusable powered applicator 4150′. In some embodiments, to prevent misalignment of the componentry, disposable assembly can include one or more alignment features (e.g., ledges, tabs, detents, slots, ridges, ribs) associated with one or more corresponding alignment features (e.g., ledges, tabs, detents, slots, ridges, ribs) of reusable powered applicator 4150′, such that the disposable assembly can only be inserted into reusable powered applicator 4150′ when the corresponding features are aligned.

According to another aspect of the embodiments, after being properly aligned, disposable assembly can be inserted into the distal end of reusable powered applicator 4150′ such that each of the one or more snaps or latches 4711 (FIG. 19R-2) engage with a corresponding ledge of reusable applicator base 4712 (not shown) inside reusable powered applicator 4150′. As the disposable assembly is inserted into the distal end of reusable powered applicator 4150′, sharp hub 4582 of sharp 4502 engages the sharp carrier retention arms of sharp carrier (not shown). According to some embodiments, sensor control device 102 can then be loaded into disposable sensor carrier 4710′ (if the sensor control device was not already part of the disposable assembly).

With respect to drive spring 4606 and sharp retraction spring 4604, it should be noted that although compression springs are shown in FIGS. 18A to 18B and 19A to 190, those of skill in the art will appreciate that other types of springs can be utilized in any of the embodiments described herein, including but not limited to torsion springs, disc springs, leaf springs and others. Furthermore, those of skill in the art will understand that the insertion and retraction speeds of the applicator embodiments described herein can be changed by changing the stiffness or length of the drive spring and the retraction spring, respectively. Similarly, those of skill in the art will understand that the timing of the sharp retraction can be modified by modifying the depth of the sheath channels (e.g., increasing depth of sheath channels can result in an earlier sharp retraction).

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, including but not limited to the powered applicator of FIGS. 18A, 18B, and 19A to 190, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

A number of deflectable structures are described herein, including but not limited to deflectable detent snaps 1402, deflectable locking arms 1412, sharp carrier lock arms 1524, sharp retention arms 1618, and module snaps 2202. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

### Example Embodiments of Applicators and Sensor Control Devices for One Piece Architectures

As previously described, certain embodiments of sensor control device 102 and applicator 150 can be provided to the user in multiple packages. For example, some embodiments, such as those described with respect to FIGS. 3A-3G, can comprise a “two-piece” architecture that requires final assembly by a user before the sensor can be properly delivered to the target monitoring location. More specifically, the sensor and the associated electrical components included in the sensor control device are provided to the user in multiple (e.g., two) packages, where each may or may not be sealed with a sterile barrier but are at least enclosed in packaging. The user must open the packaging and follow instructions to manually assemble the components and subsequently deliver the sensor to the target monitoring location with the applicator. For example, referring again to FIGS. 3A-3G, the sensor tray and applicator are provided to the user as separate packages, thus requiring the user to open each package and finally assembly the system. In some applications, the discrete, sealed packages allow the tray and the applicator to be sterilized in separate sterilization processes unique to the contents of each package and otherwise incompatible with the contents of the other.

More specifically, the tray, which includes a plug assembly, including the sensor and sharp, may be sterilized using radiation sterilizations, such as electron beam (or “e-beam”) irradiation. Radiation sterilization, however, can damage the electrical components arranged within the housing of the sensor control device. Consequently, if the applicator, which contains the housing of the sensor control device, needs to be sterilized, it may be sterilized via another method, such as gaseous chemical sterilization using, for example, ethylene oxide. Gaseous chemical sterilization, however, can damage the enzymes or other chemistry and biologics included on the sensor. Because of this sterilization incompatibility, the tray and applicator may be sterilized in separate sterilization processes and subsequently packaged separately, and thereby require the user to finally assembly the components upon receipt.

According to other embodiments of the present disclosure, the sensor control device (e.g., analyte sensor device) may comprise a one-piece architecture that incorporates sterilization techniques specifically designed for a one-piece architecture. The one-piece architecture allows the sensor control device assembly to be shipped to the user in a single, sealed package that does not require any final user assembly steps. Rather, the user need only open one package and subsequently deliver the sensor control device to the target monitoring location. The one-piece system architecture described herein may prove advantageous in eliminating component parts, various fabrication process steps, and user assembly steps. As a result, packaging and waste are reduced, and the potential for user error or contamination to the system is mitigated.

According to some embodiments, a sensor sub-assembly (SSA) can be built and sterilized. The sterilization may be, for example, radiation, such as electron beam (e-beam radiation), but other methods of sterilization may alternatively be used including, but not limited to, gamma ray radiation, X-ray radiation, or any combination thereof. Embodiments of methods of manufacturing an analyte monitoring system using this SSA are now described, as are embodiments of sensor control devices having this SSA and applicators for use therewith. An SSA can be manufactured and then sterilized. During sterilization the SSA can include both an analyte sensor and an insertion sharp. The sterilized SSA can then be assembled to form (e.g., assembled into) a sensor control device, e.g., the sterilized SSA can be placed such that the sensor is in electrical contact with any electronics in a sensor carrier. This sensor control device can then be assembled to form (e.g., assembled into) an applicator (e.g., as a one-piece assembly) where the applicator (also referred to as an analyte sensor inserter) is configured to apply the sensor control device to a user's body. The one-piece assembly can be packaged and/or distributed (e.g., shipped) to a user or health care professional.

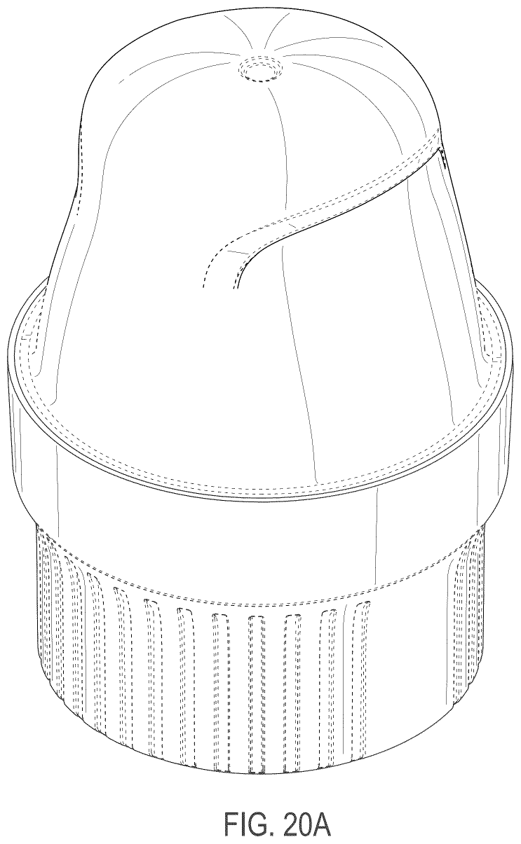

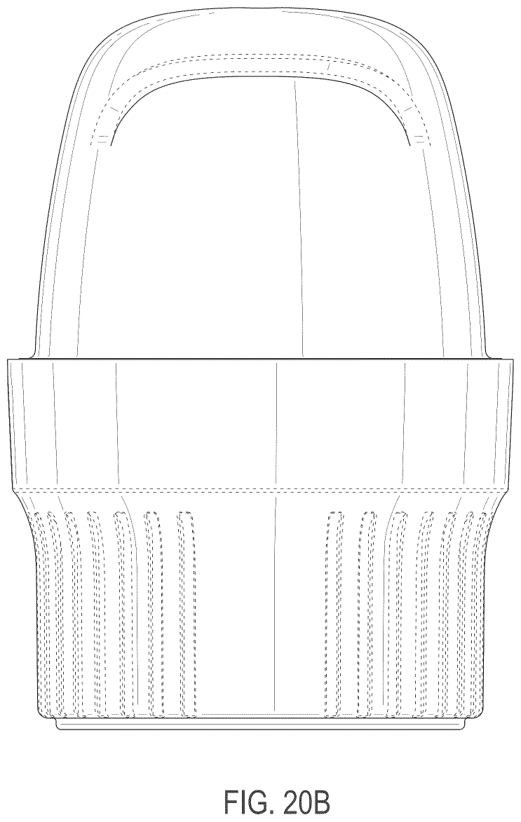

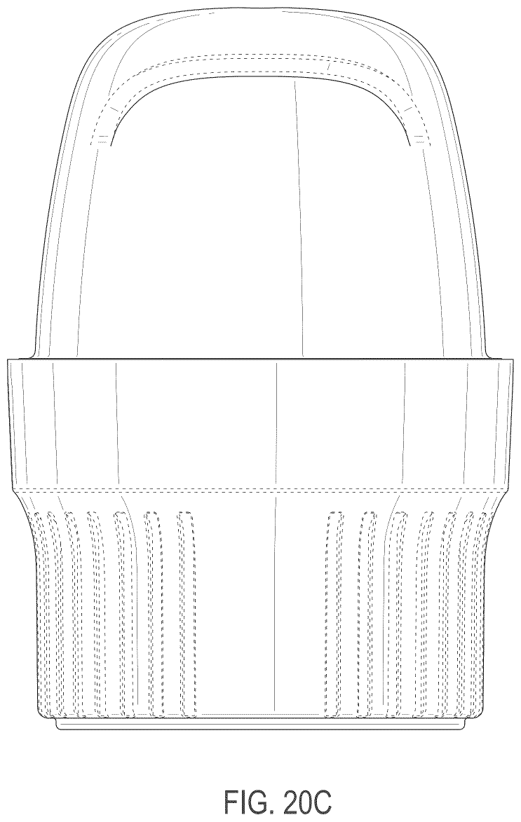

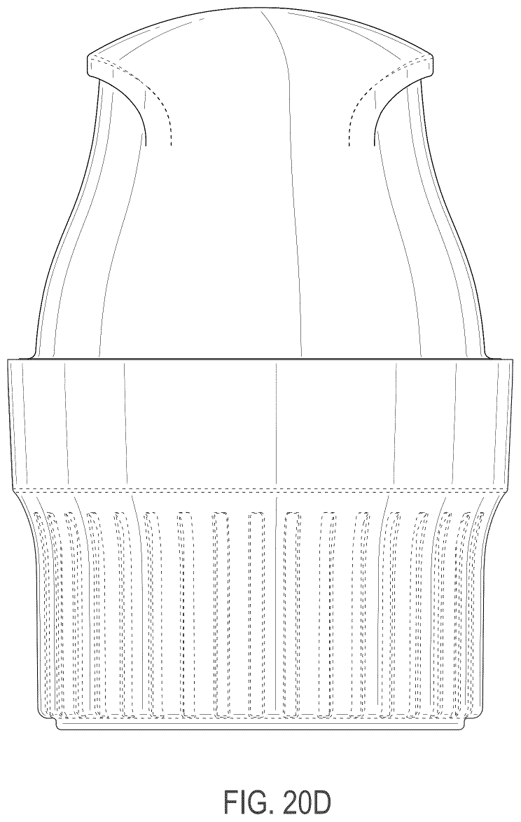

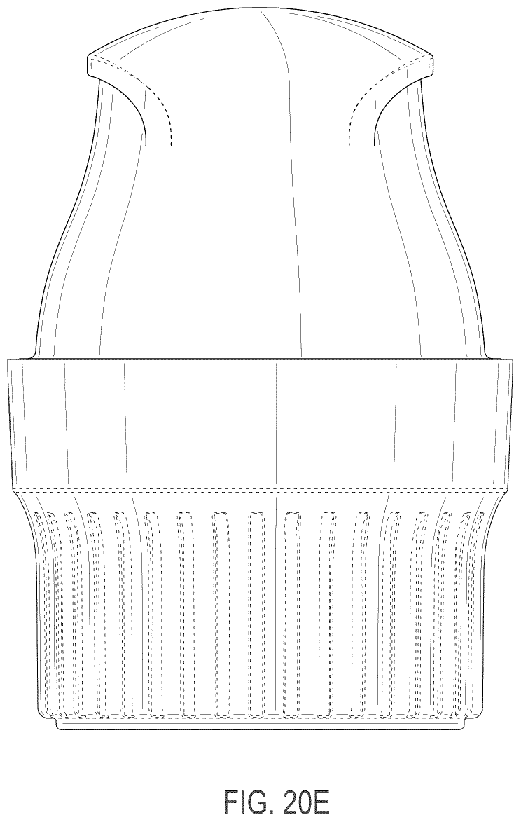

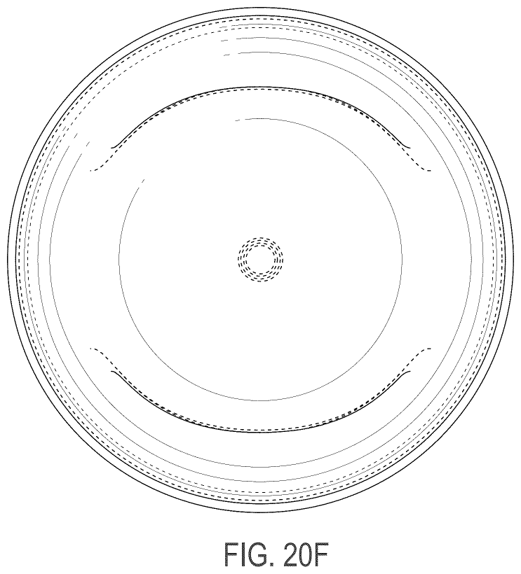

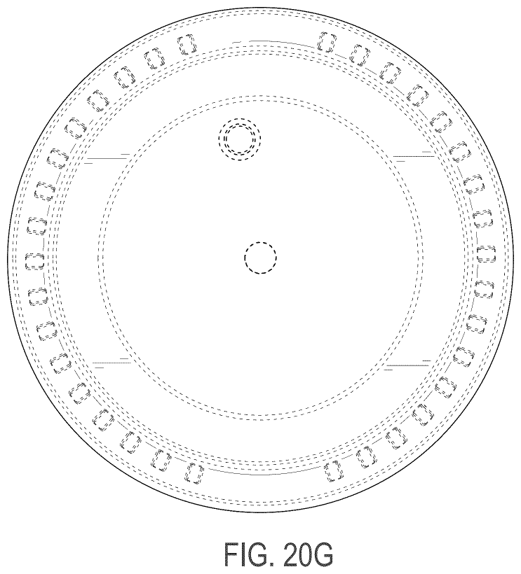

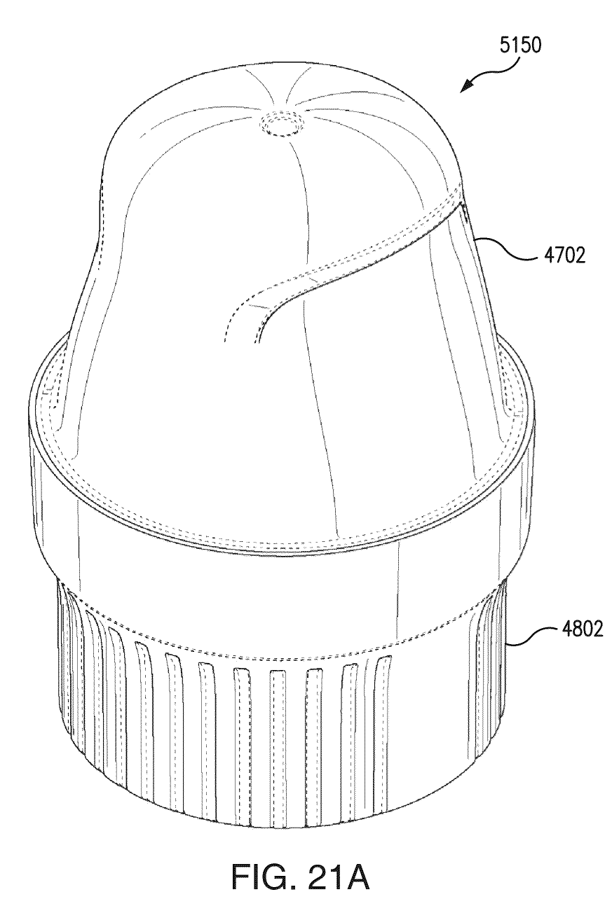

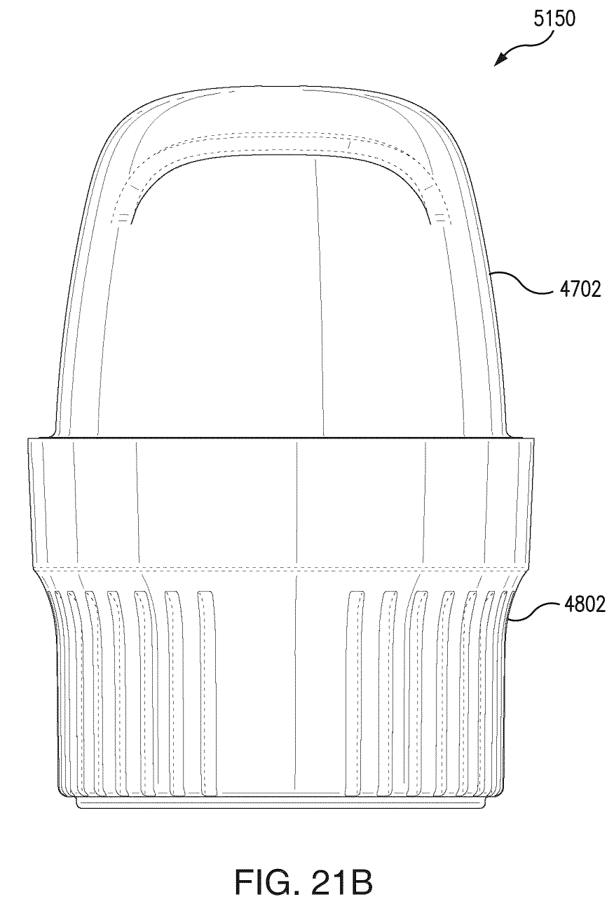

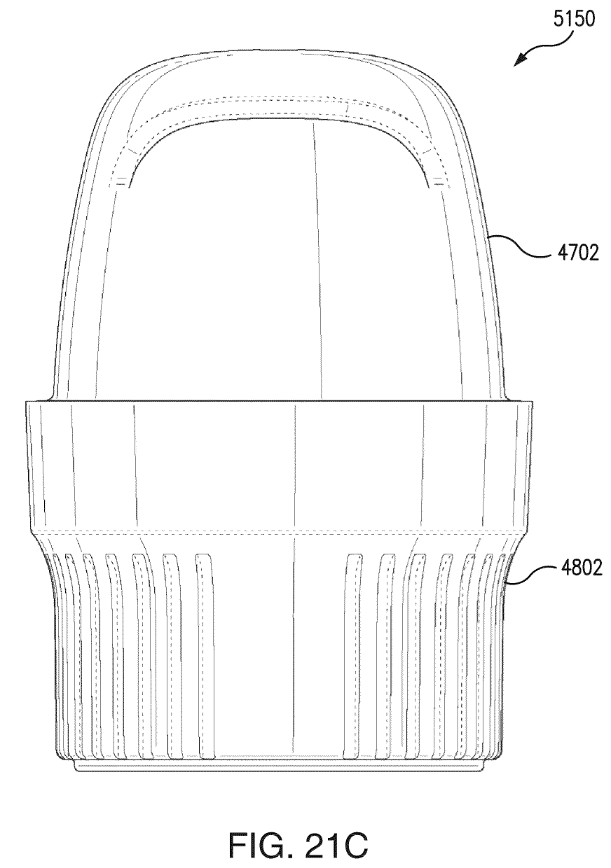

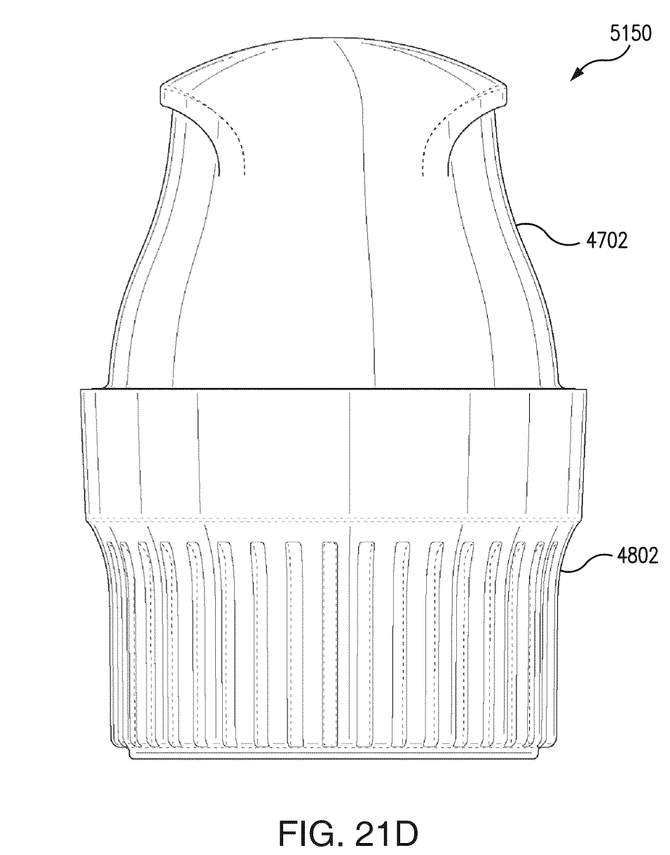

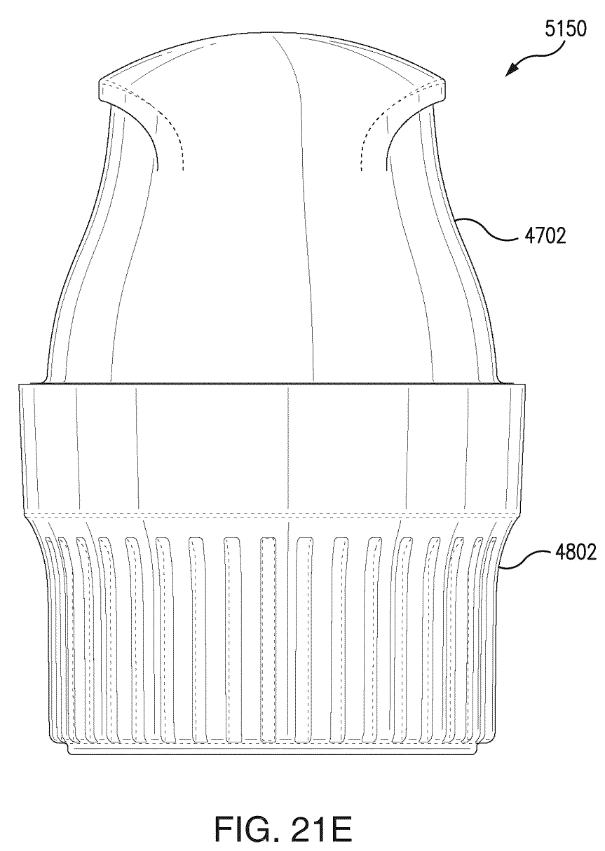

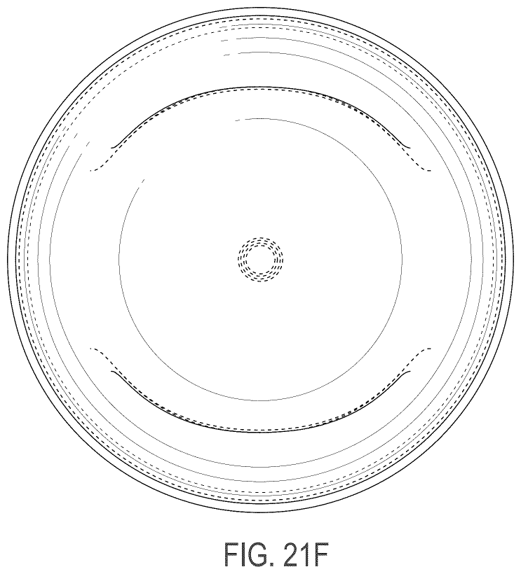

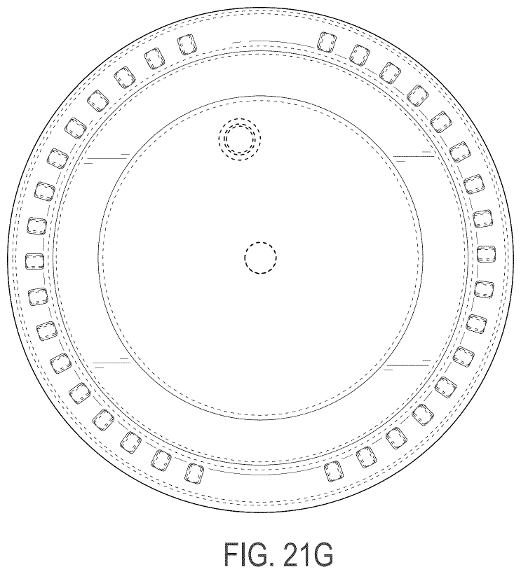

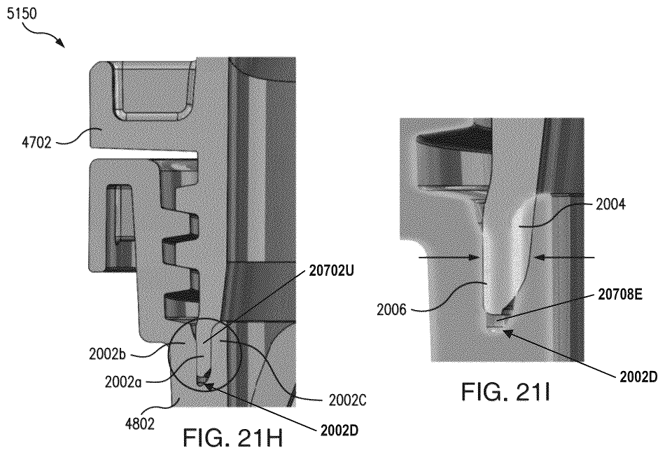

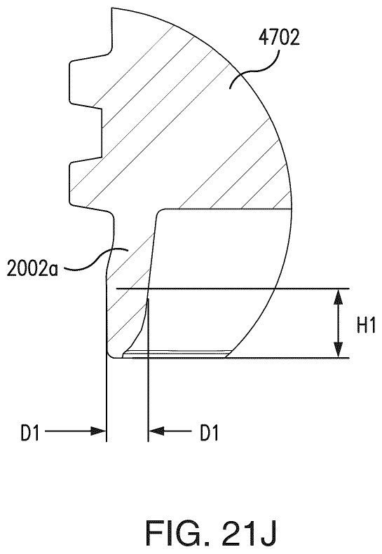

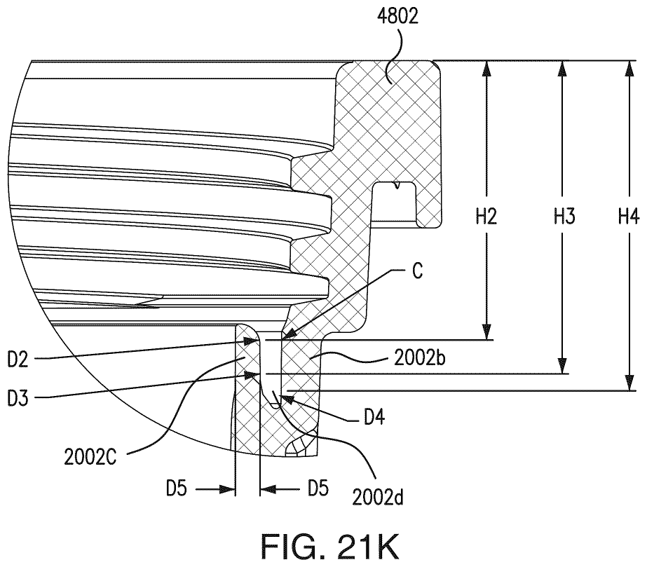

FIGS. 20A-20G depict a first embodiment of a one-piece applicator for use with a sensor control device having an SSA. FIGS. 21A-21G depict a second embodiment of the one-piece applicator for use with a sensor control device having an SSA. As can be seen in FIGS. 21A-21E, one-piece applicator 5150 can include housing 4702 and applicator cap 4802 mateable with housing 4702. Applicator cap 4802 provides a barrier that protects the internal contents of one-piece applicator 5150. In some embodiments, applicator cap 4802 may be secured to housing 4702 by a threaded engagement and, upon rotating (e.g., unscrewing) applicator cap 4802 relative to housing 4702, applicator cap 4802 can be freed from housing 4702. In other embodiments, however, applicator cap 4802 may be secured to housing 4702 via an interference or shrink fit engagement. Consequently, to use one-piece applicator 210 for insertion of an analyte sensor, user can remove applicator cap 210 from housing 208. Furthermore, although not depicted, one-piece applicator 5150 can also include any of the embodiments of powered applicators, sensor control units, analyte sensors, and sharps described herein, or in other publications which have been incorporated by reference.

As described herein below, the coupled engagement between housing 4702 and applicator cap 4802 can provide sterility to the components positioned within one-piece applicator 5150 by maintaining a sterile environment as sealed with applicator cap 4802. The embodiments described herein below may be applicable to analyte monitoring systems that incorporate a two-piece or a one-piece architecture. More particularly, in embodiments employing a two-piece architecture, the electronics housing (not shown) that retains the electrical components for sensor control device 102 (FIG. 1) may be positioned within housing 4702 and applicator cap 4802 maintains the sterile environment. In contrast, in embodiments employing a one-piece architecture, one-piece applicator 5150 may contain the fully assembled sensor control device 102 (FIG. 1), and applicator cap 4802 maintains the sterile environment for the fully assembled sensor control device.

FIGS. 21H-K show an enlarged cross-sectional side view of the interface between housing 4702 and applicator cap 4802. As illustrated, applicator cap sealing lip 20702U of housing 4702 includes a first axial extension 2002a, and seal interface 20708E of applicator cap 4802 provides a cavity 2002d mateable with the first axial extension 2002a. In the illustrated embodiment, the diameter of cavity 2002d formed from second axial extension 2002b and third axial extension 2002c of the applicator cap 4802 is sized to receive the diameter of first axial extension 2002a of housing 4702 within cavity 2002d. For example, as shown in FIG. 21J, axial extension 2002a can have thickness D1 at height H1, as measured from distal edge of axial extension 2002a. Similarly, second axial extension 2002c can have a thickness D5 at height H3, as measured from proximal edge of applicator cap 210; cavity 2002d can have a thickness D2, D3, and D4 at heights H2, H3, and H4, respectively, as measured from proximal edge of applicator cap 210. In certain embodiments, D1 can measure 1 mm with a tolerance of +/−0.03 mm, D2, D3, D4 can have any suitable dimensions, H1 can measure 1.66 mm with a tolerance of +/−0.1 mm, H2 can measure 8.25 mm with a tolerance of +/−0.1 mm, H3 can measure 9.25 mm with a tolerance of +/−0.1 mm, H4 can measure 9.75 mm with a tolerance of +/−0.1 mm. In other embodiments, however, the reverse can be employed, where the diameter of first axial extension 2002a can be sized to receive the diameter of the second axial extension 2002b, without departing from the scope of the disclosure.

In each embodiment, two radial seals 2004, 2006 can be defined or otherwise provided at the interface between first and second axial extensions 2002a, b and radial seals 2004 and 2006 may help prevent migration of fluids or contaminants across the interface in either axial direction. Moreover, the dual radial seals described herein can accommodate tolerance and thermal variations combined with stress relaxation via a redundant sealing strategy. In the illustrated embodiment, dual radial seals 2004, 2006 utilize a “wedge” effect for effective sealing between first axial extension 2002a and second axial extension 2002b.

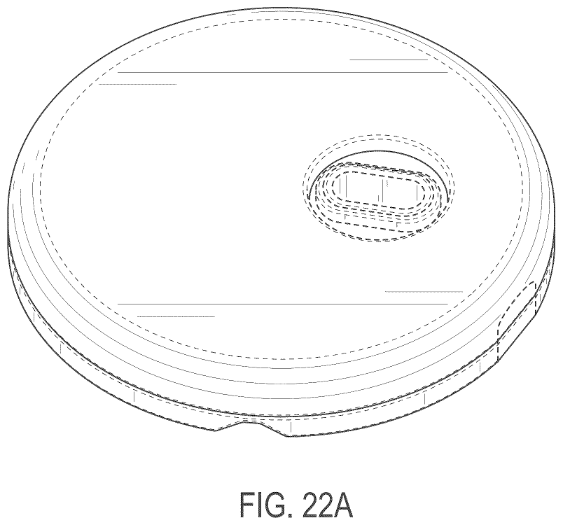

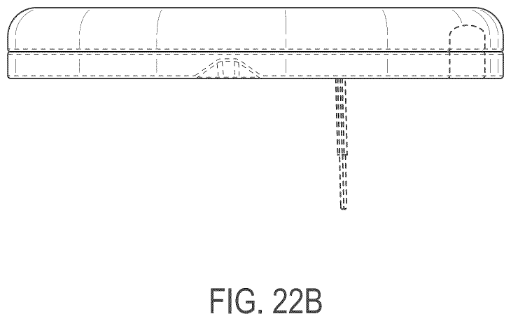

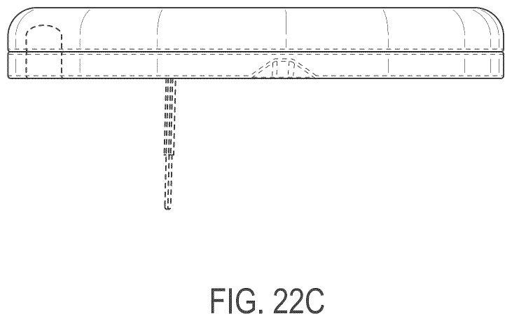

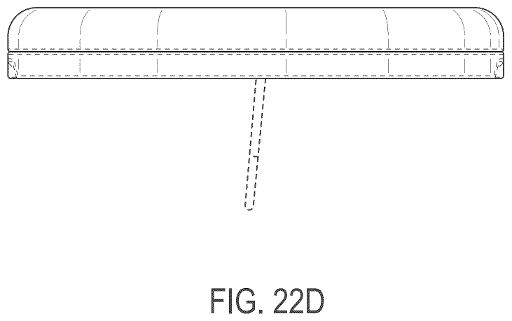

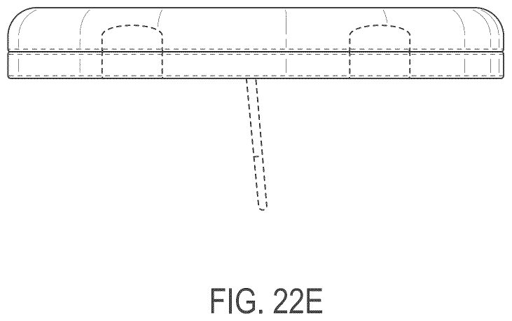

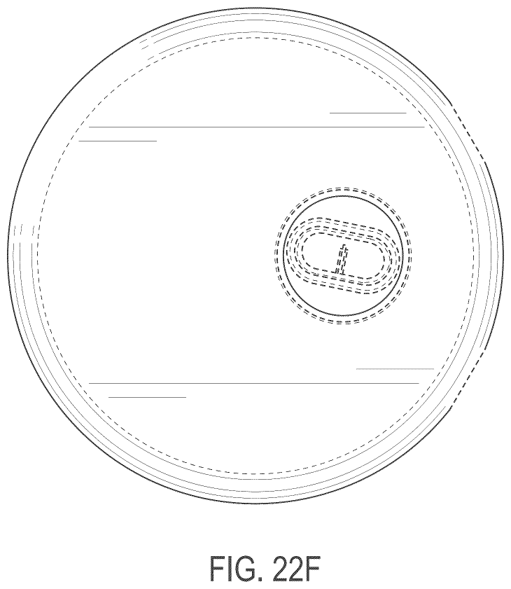

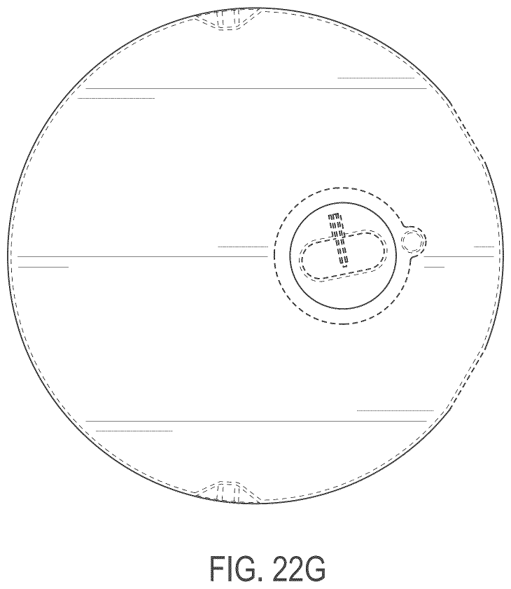

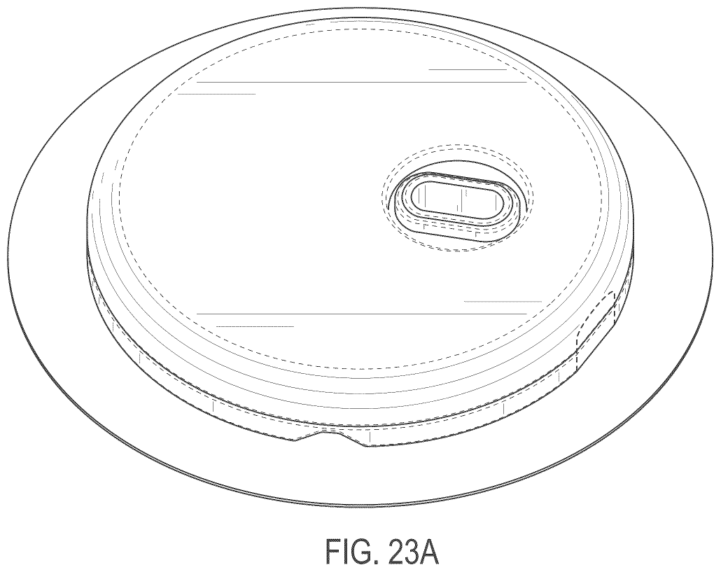

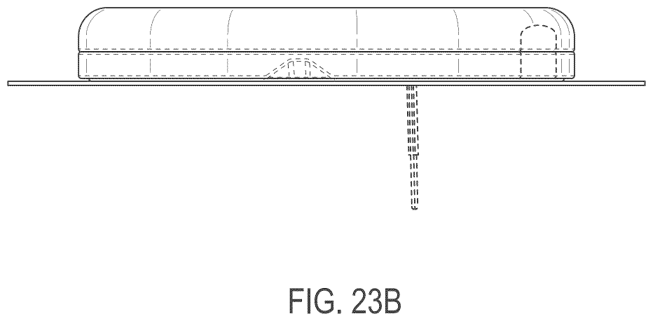

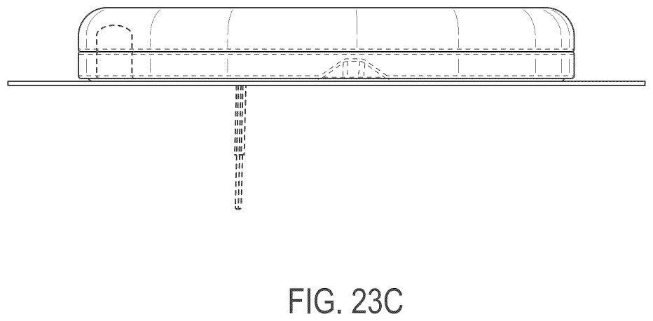

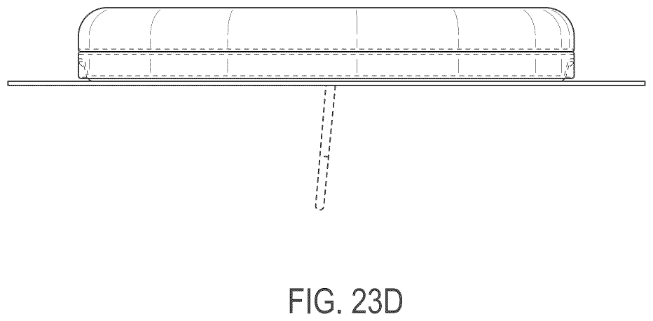

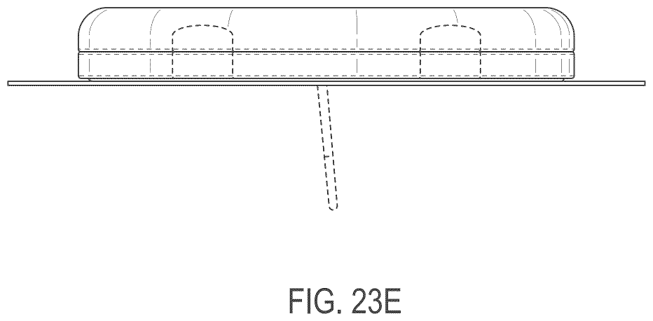

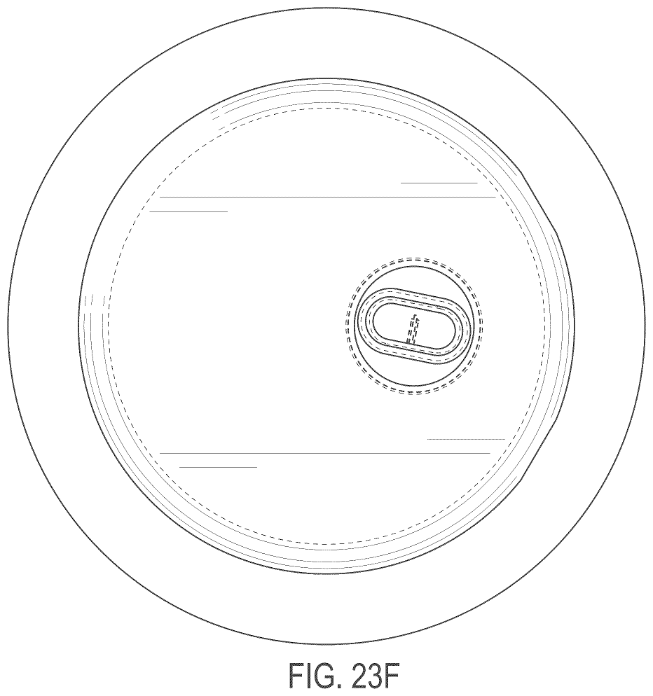

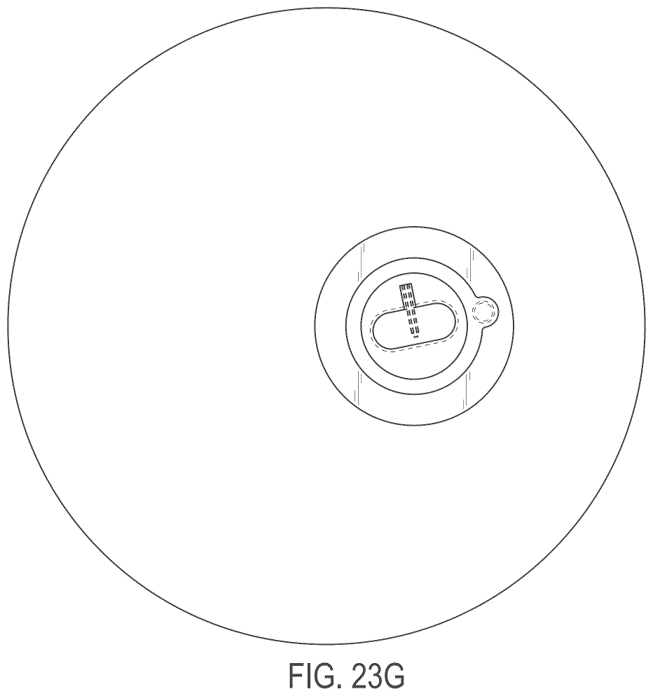

FIGS. 22A-22G depict a first embodiment of a sensor control device having an SSA but without an adhesive patch. FIGS. 23A-23G depict a second embodiment of the sensor control device having an SSA and an adhesive patch.

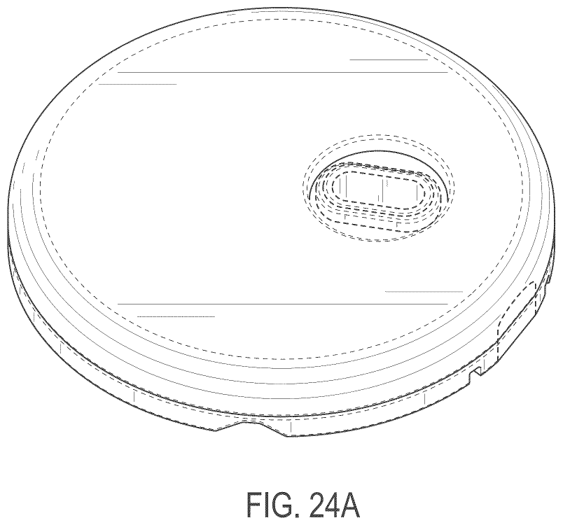

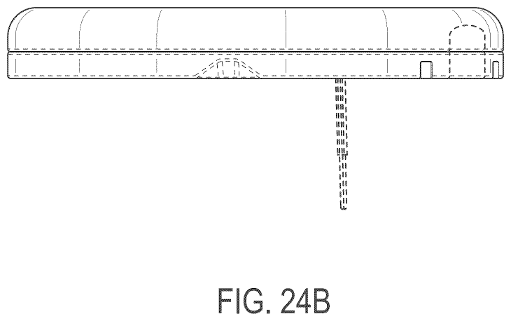

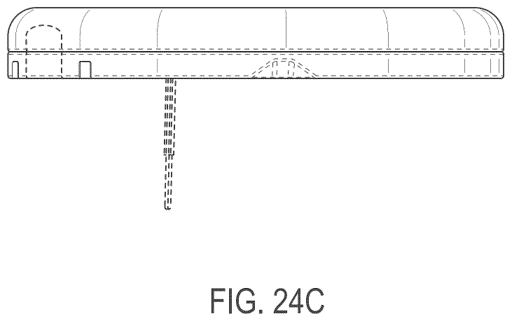

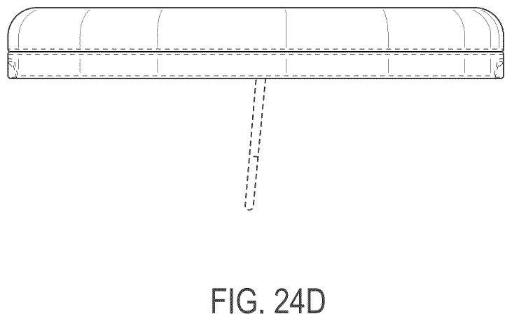

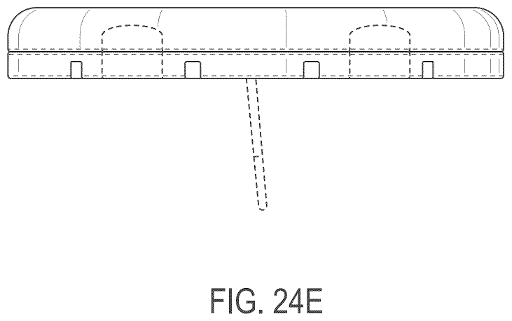

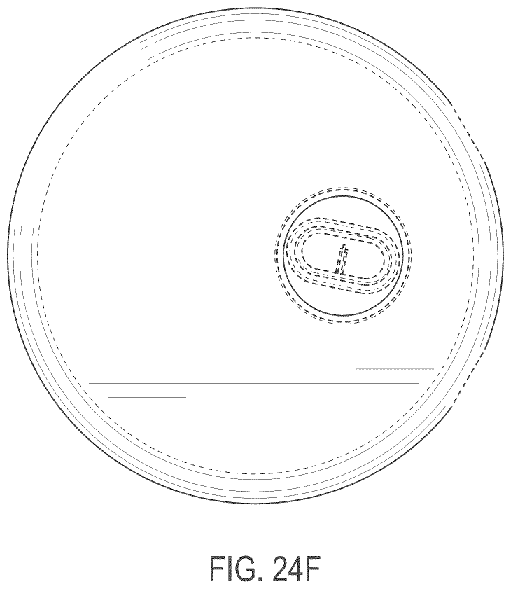

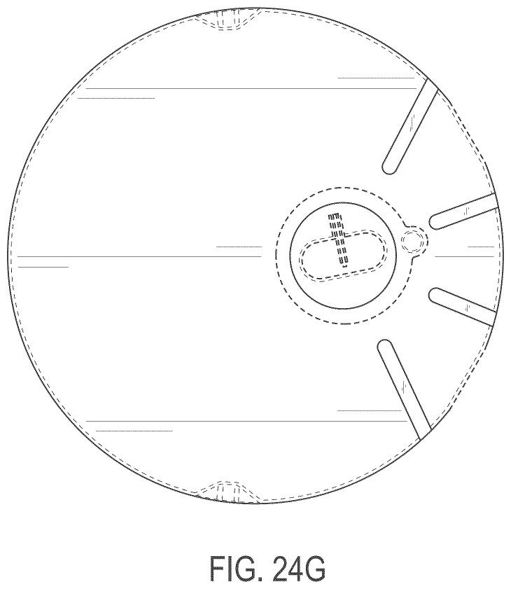

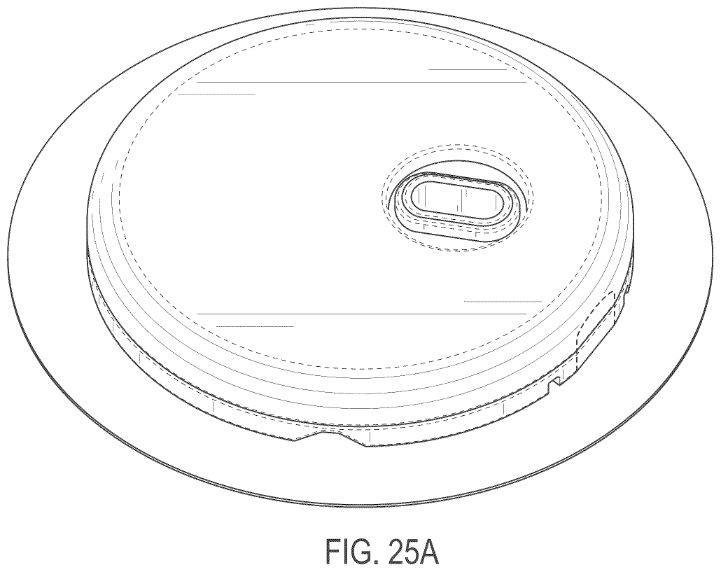

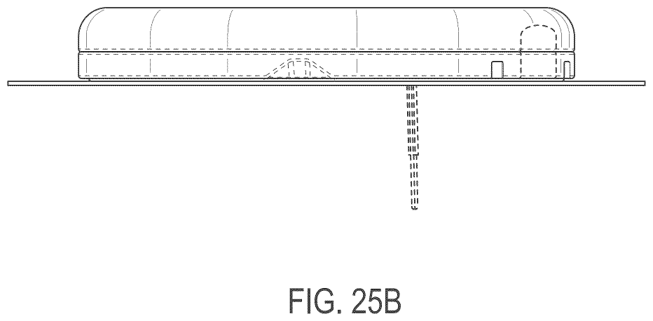

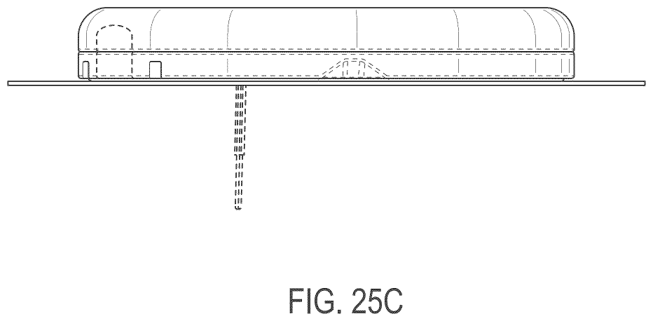

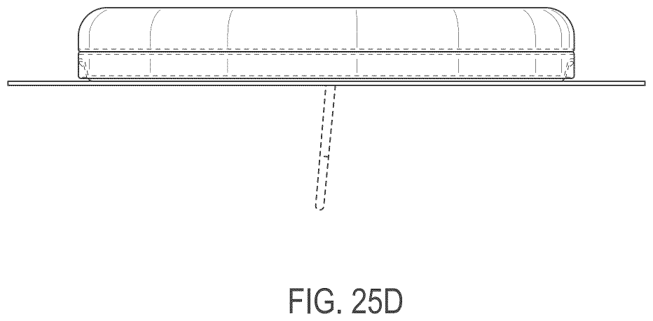

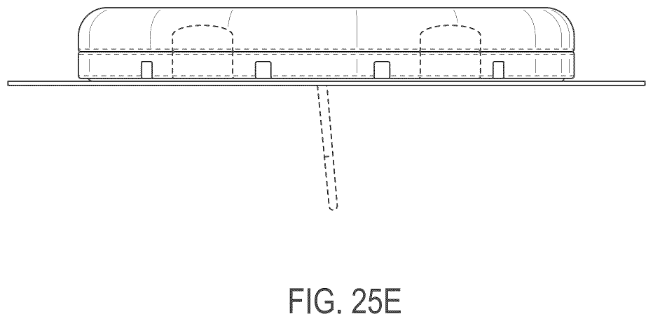

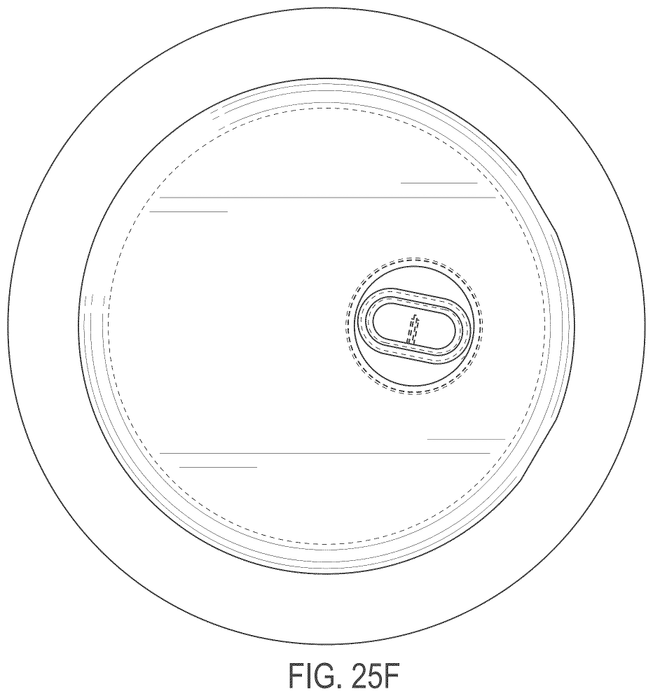

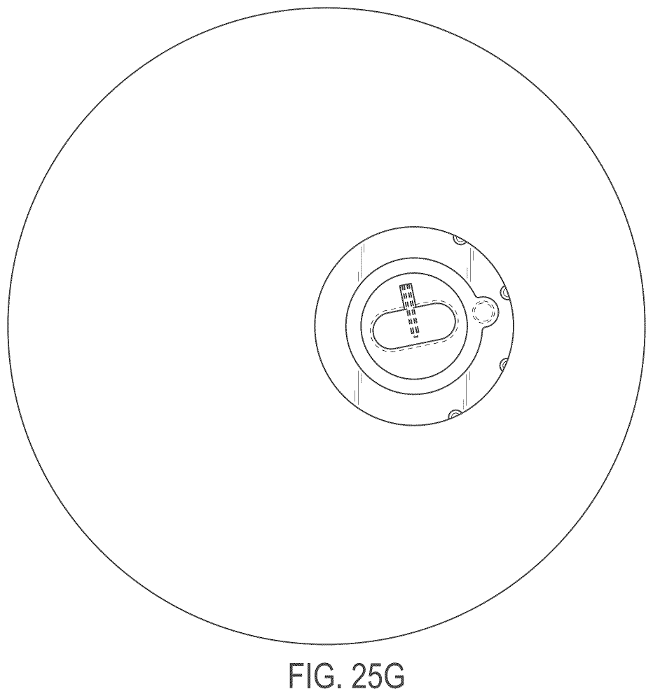

FIGS. 24A-24G depict a third embodiment of a sensor control device having an SSA and bottom surface grooves, but without an adhesive patch. FIGS. 25A-25G depict a fourth embodiment of the sensor control device having an SSA, bottom surface grooves, and an adhesive patch.

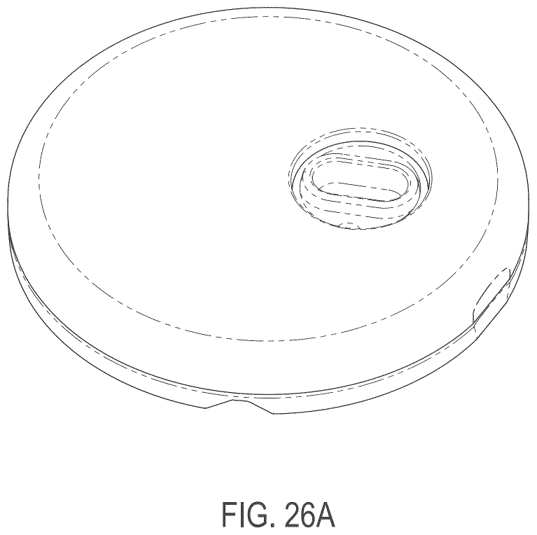

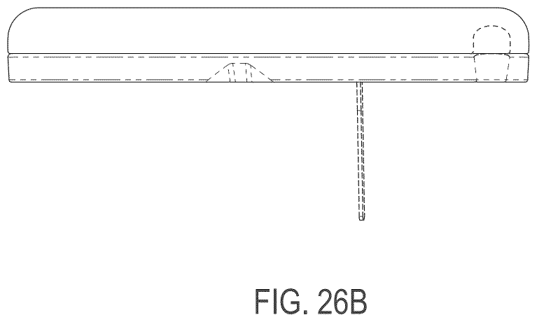

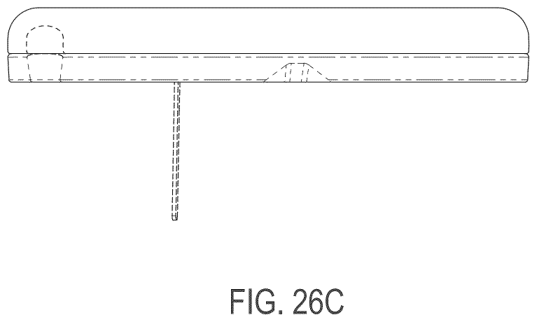

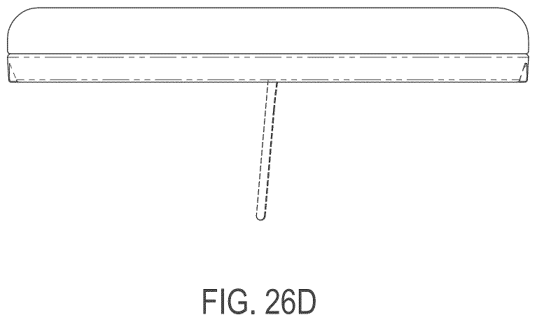

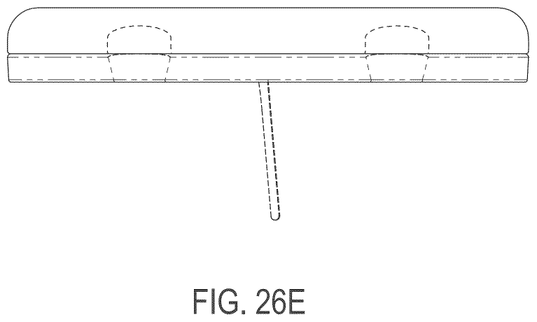

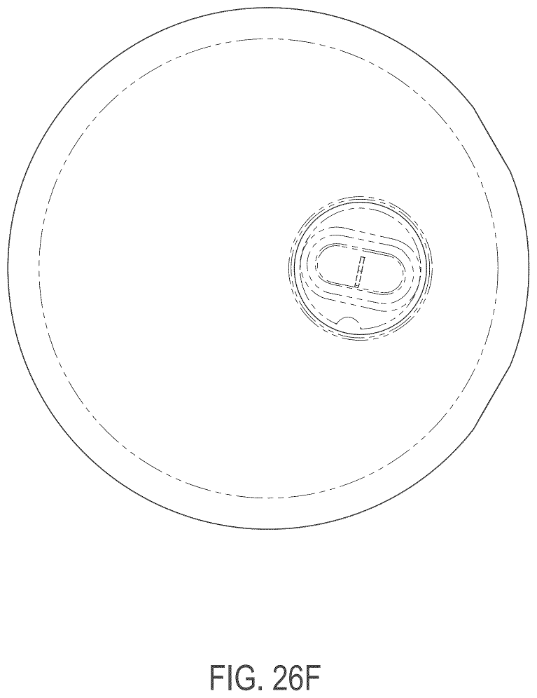

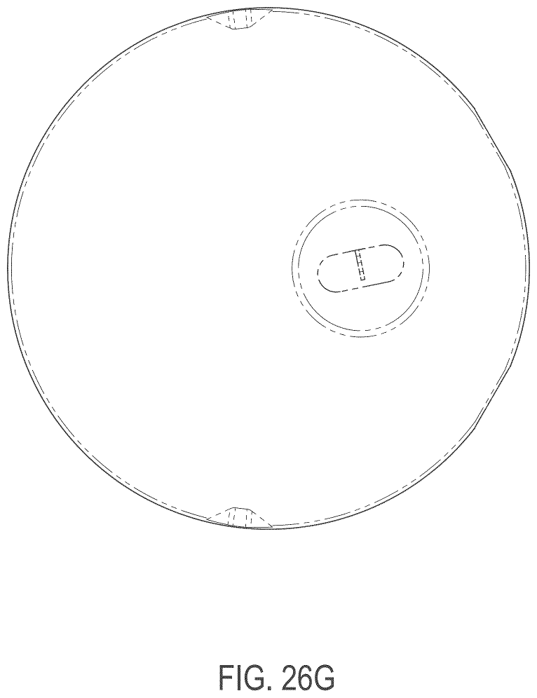

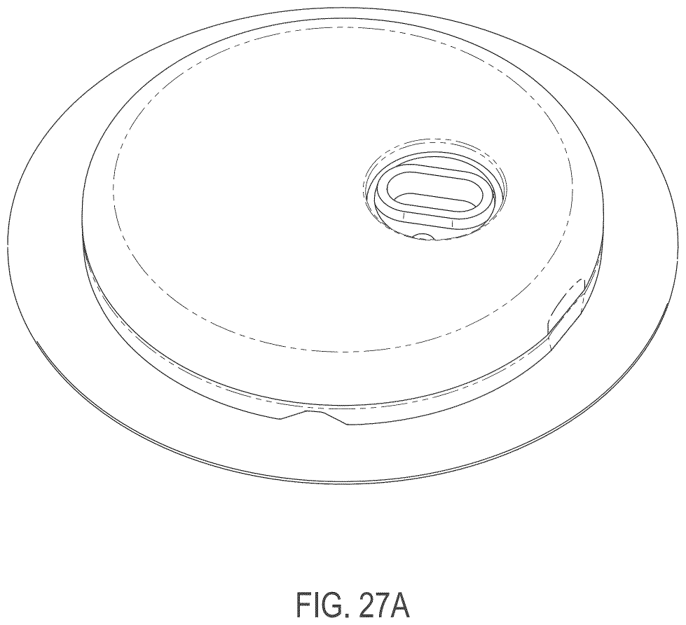

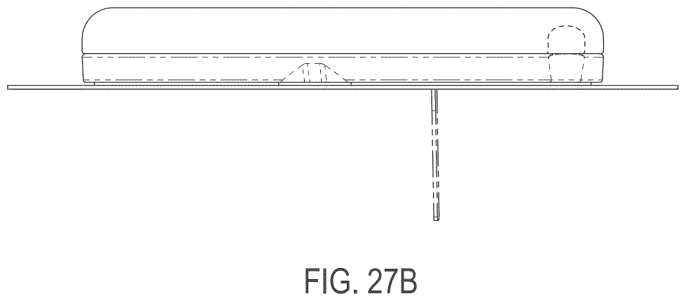

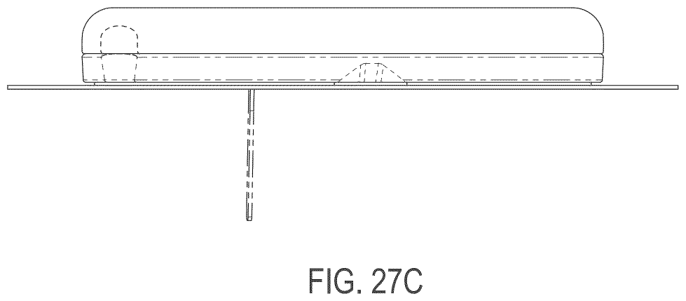

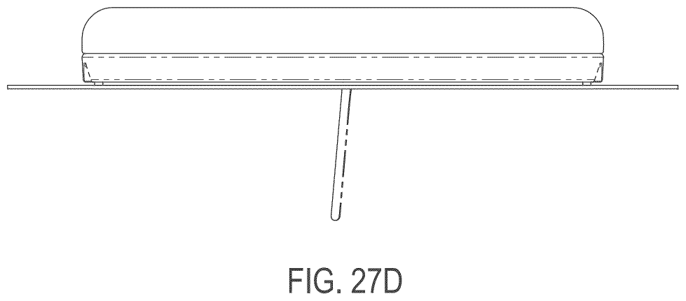

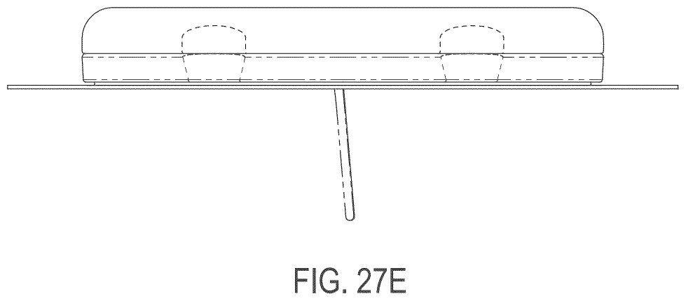

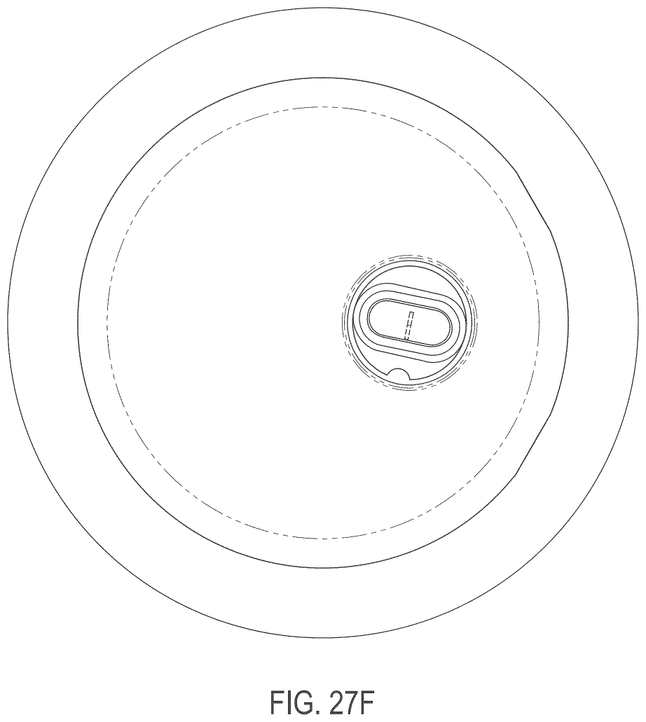

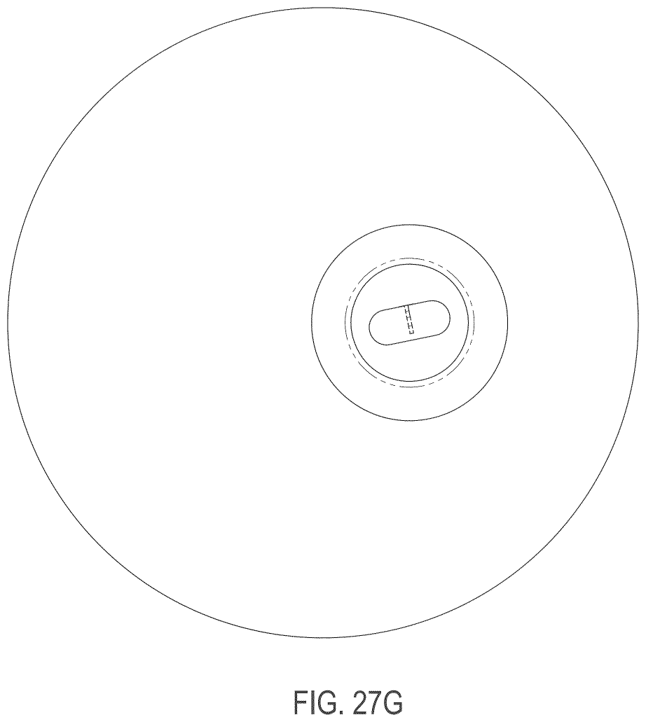

FIGS. 26A-26G depict a fifth embodiment of a sensor control device having an SSA but without an adhesive patch. FIGS. 27A-27G depict a sixth embodiment of the sensor control device having an SSA and an adhesive patch.

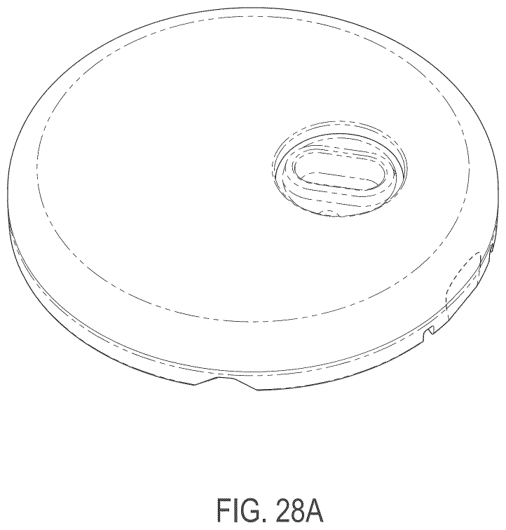

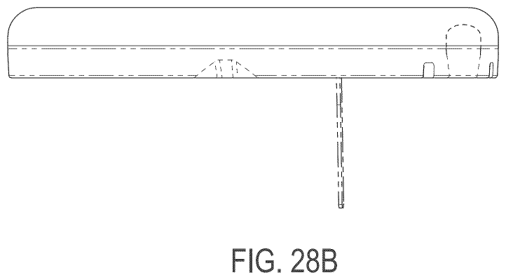

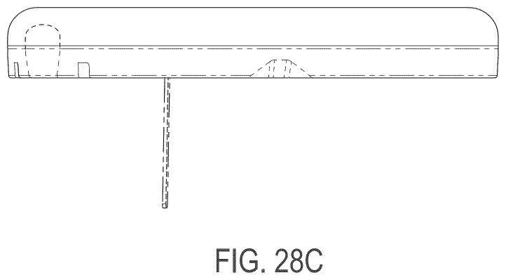

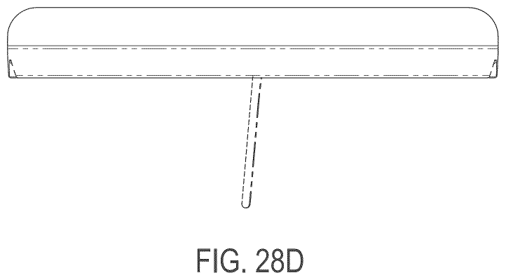

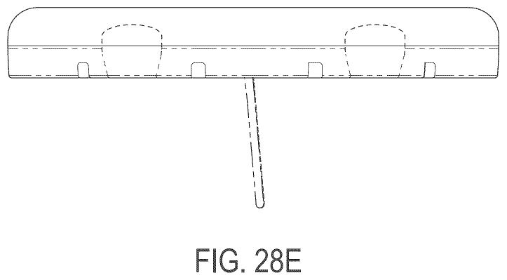

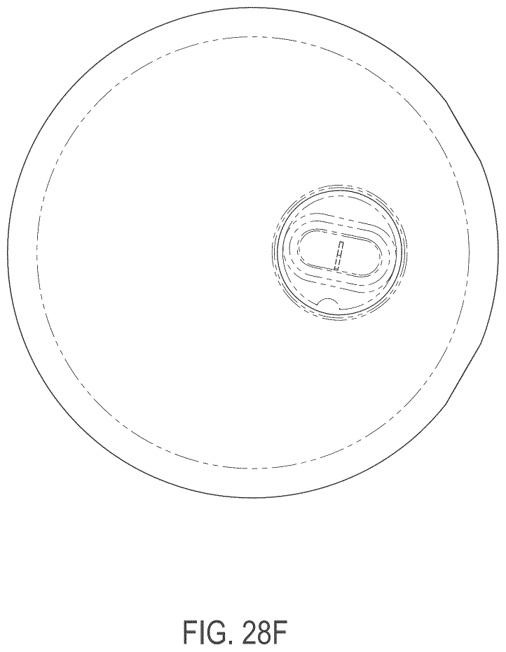

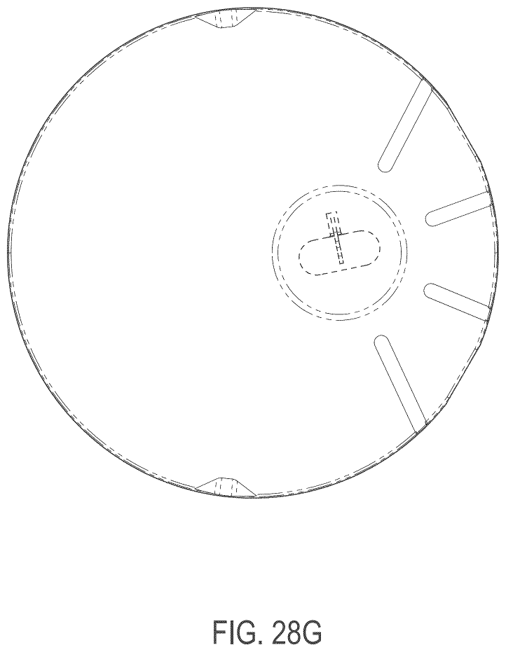

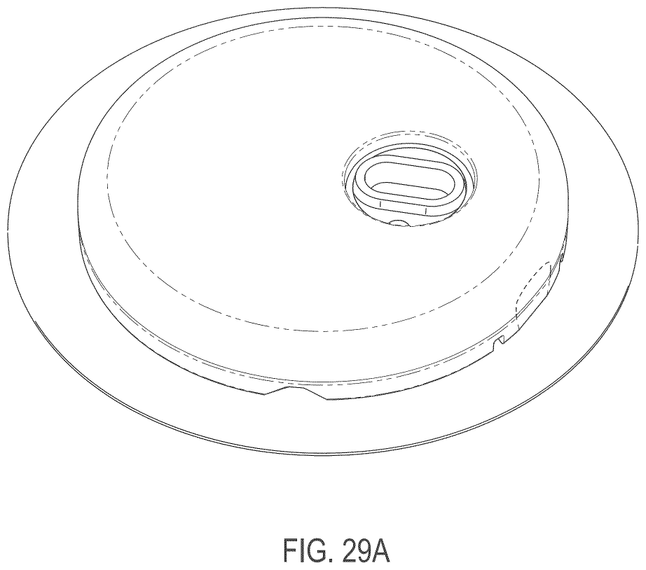

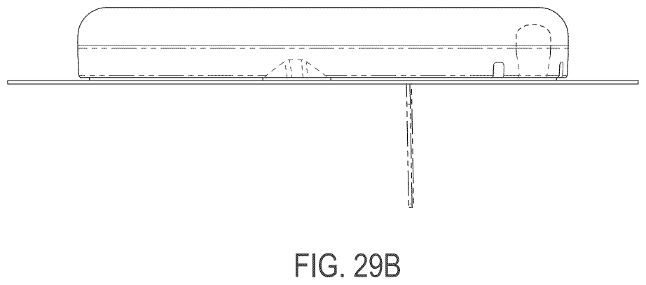

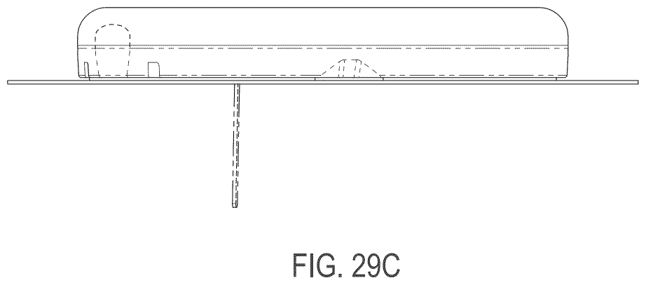

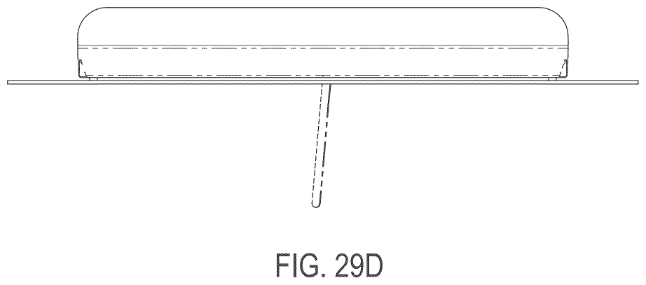

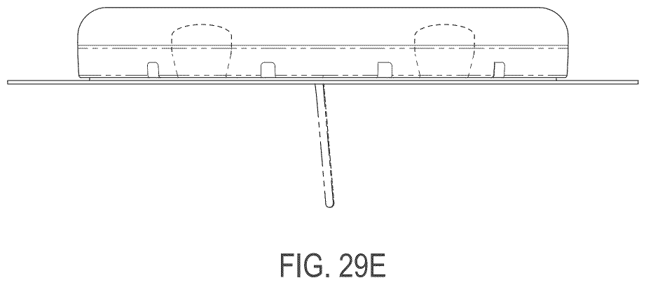

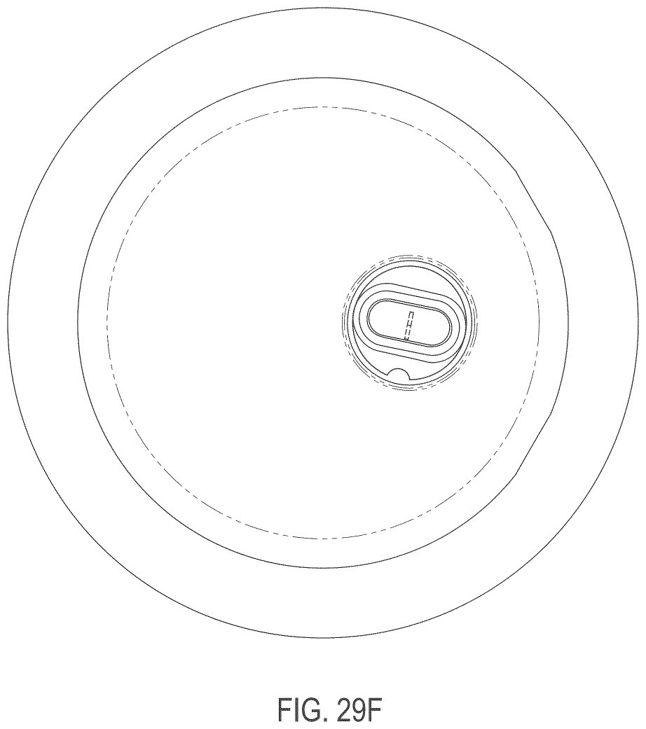

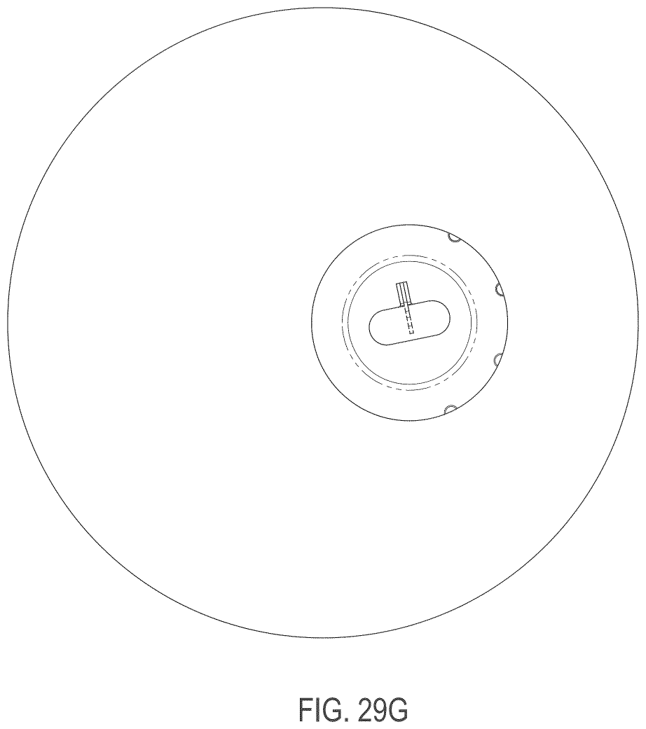

FIGS. 28A-28G depict a seventh embodiment of a sensor control device having an SSA and bottom surface grooves, but without an adhesive patch. FIGS. 29A-29G depict an eight embodiment of the sensor control device having an SSA, bottom surface grooves, and an adhesive patch.

Additional details of suitable devices, systems, methods, components and the operation thereof along with related features are set forth in International Publication No. WO2018/136898 to Rao et. al., International Publication No. WO2019/236850 to Thomas et. al., International Publication No. WO2019/236859 to Thomas et. al., International Publication No. WO2019/236876 to Thomas et. al., and U.S. patent application Ser. No. 16/433,931, filed Jun. 6, 2019, each of which is incorporated by reference in its entirety herein.

According to other embodiments, the sensor control device, including a battery and sensor, can be built into the applicator as a one-piece assembly, and sterilized using a focused electron beam (FEB). Other methods of sterilization may alternatively be used including, but not limited to, gamma ray radiation, X-ray radiation, or any combination thereof. Embodiments of methods of manufacturing an analyte monitoring system and sterilizing with, for example, an FEB are now described, as are embodiments of sensor control devices and applicators for use therewith. A sensor control device including a sensor and a sharp can be manufactured or assembled, e.g., the sensor can be placed in electrical contact with any electronics in a sensor carrier of the sensor control device. This sensor control device can then be assembled to form (e.g., assembled into) an applicator (e.g., as a one-piece assembly) where the applicator is configured to apply the sensor control device to a user's body. This assembled applicator, having the sensor control device therein, can then be sterilized with, for example, an FEB. The sterilized applicator can then be packaged and/or distributed (e.g., shipped) to a user or health care professional. In some embodiments a desiccant and foil seal can be added to the sterilized one-piece assembly prior to packaging.

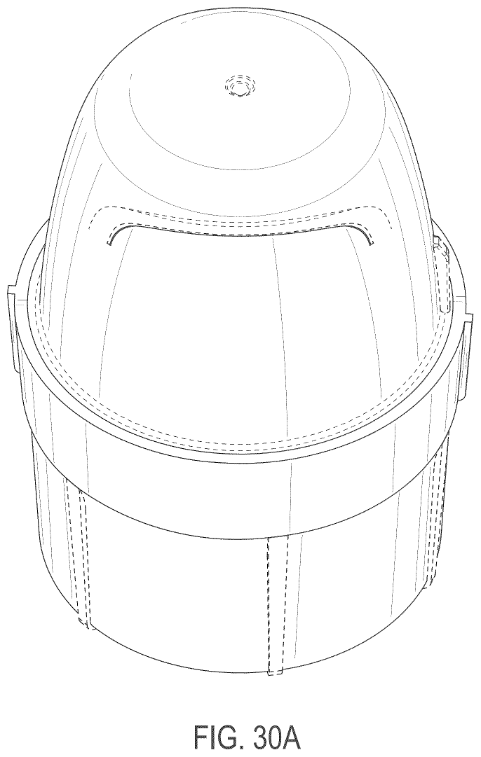

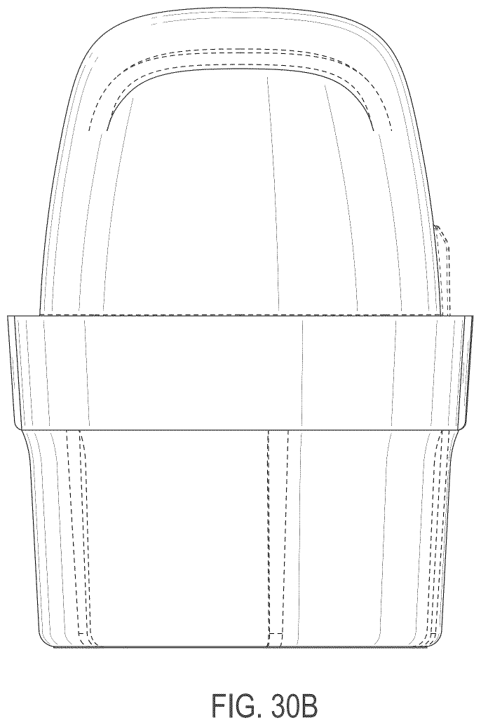

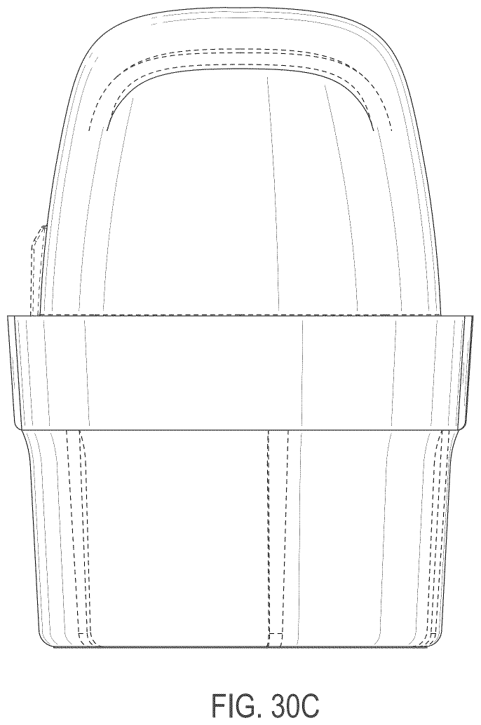

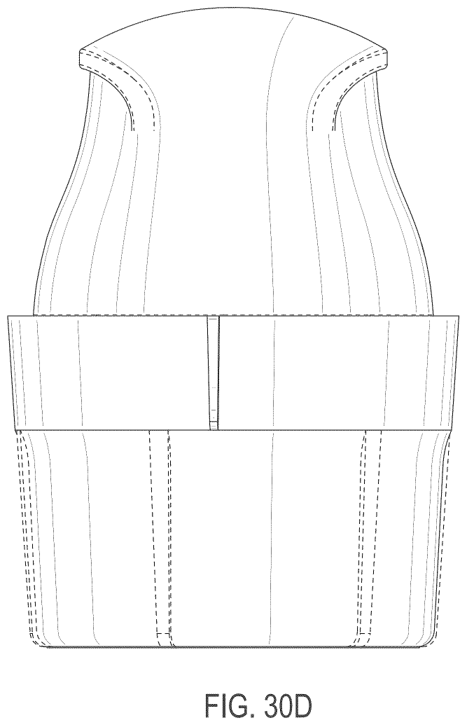

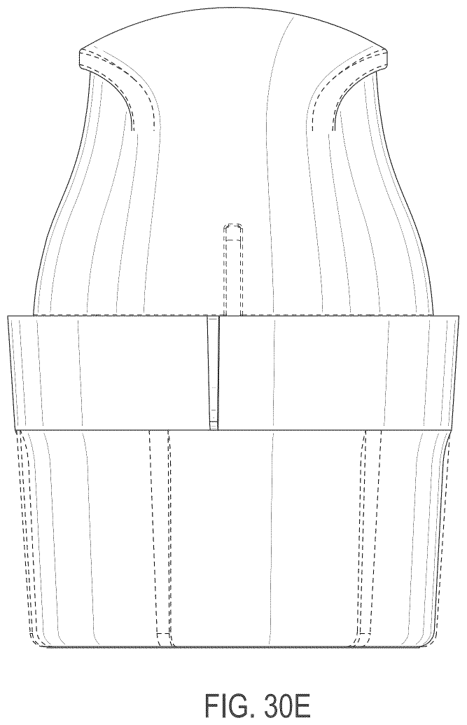

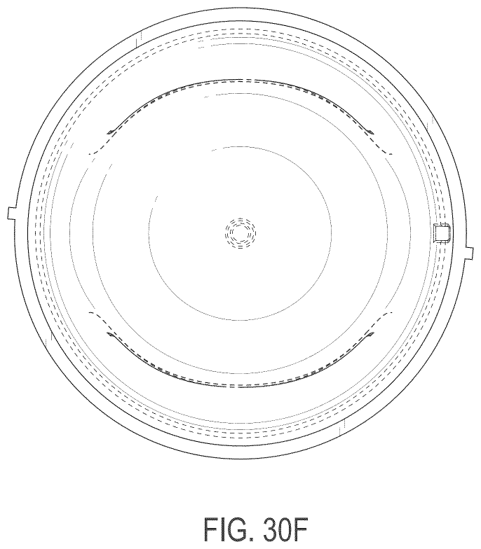

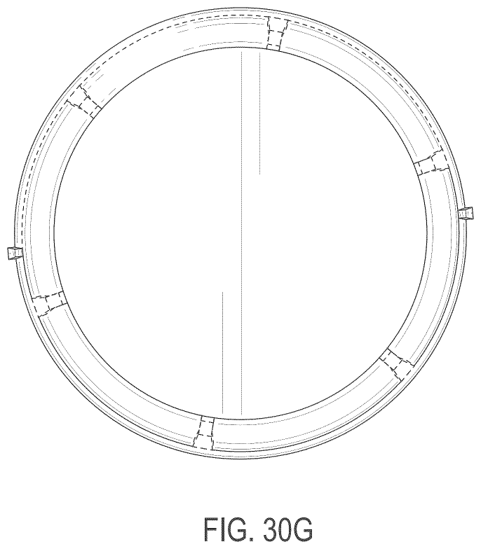

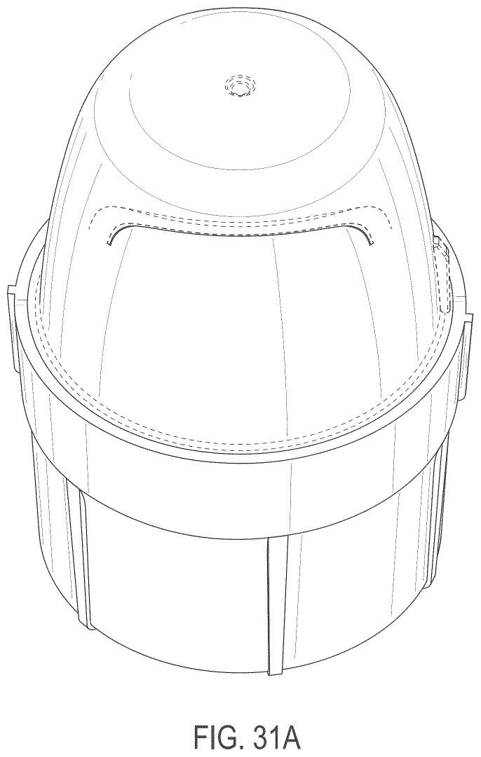

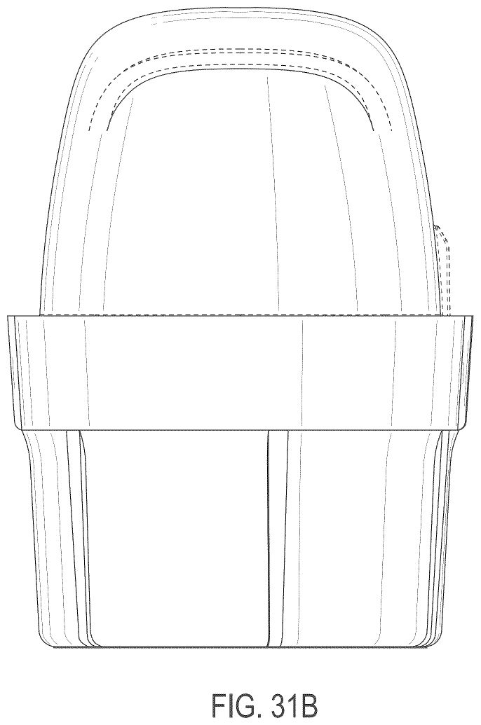

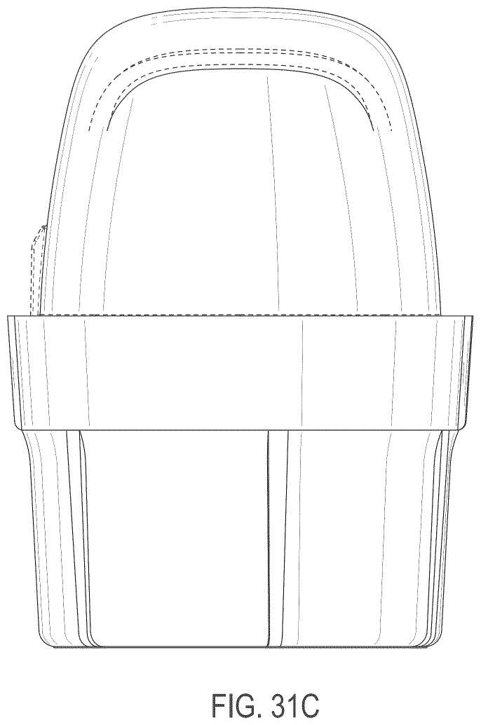

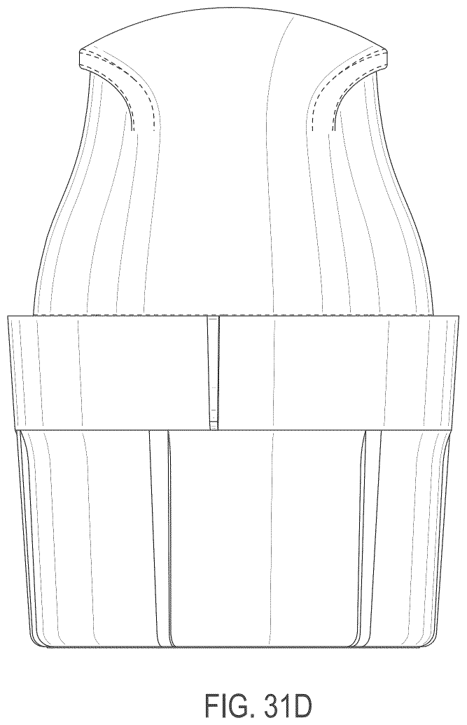

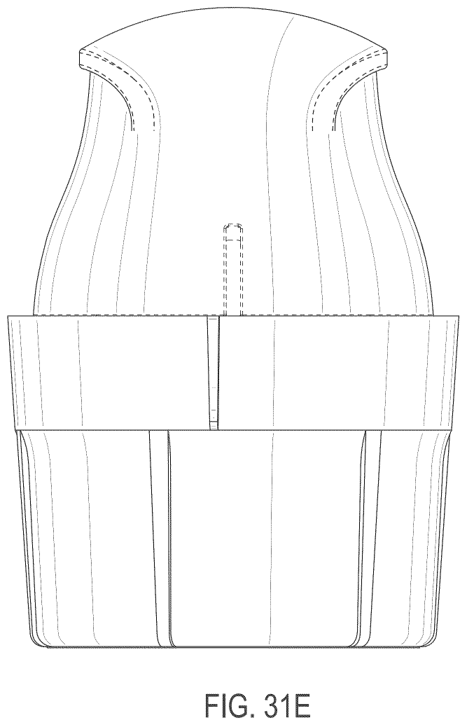

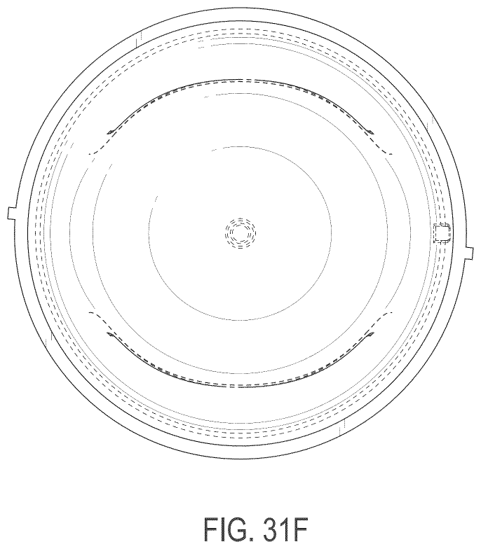

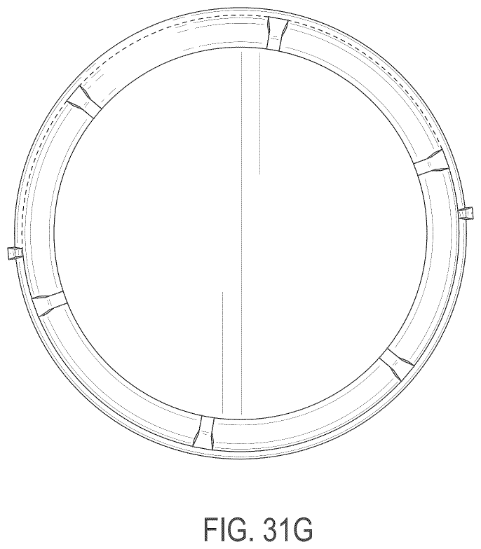

FIGS. 30A-30G depict a first embodiment of an applicator for sterilization with, e.g., an FEB. FIGS. 31A-31G depict a second embodiment of the applicator for sterilization with, e.g., an FEB.

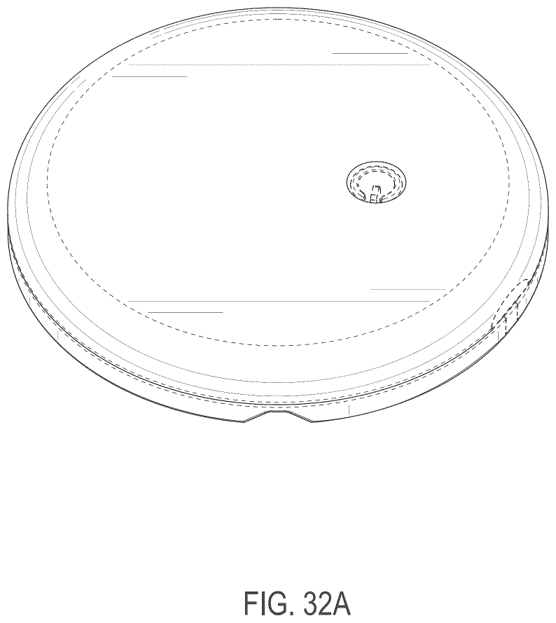

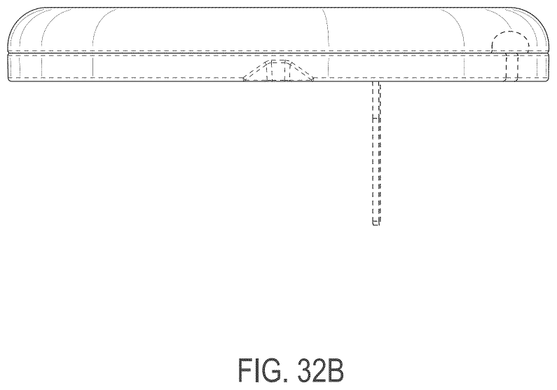

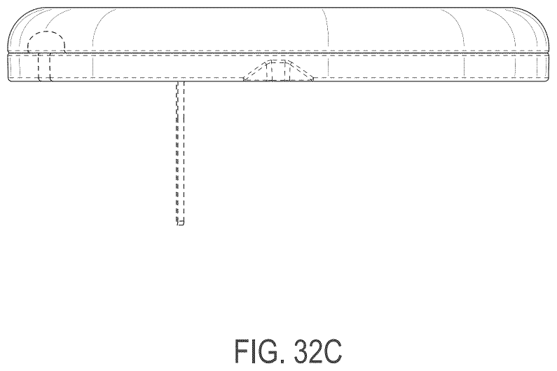

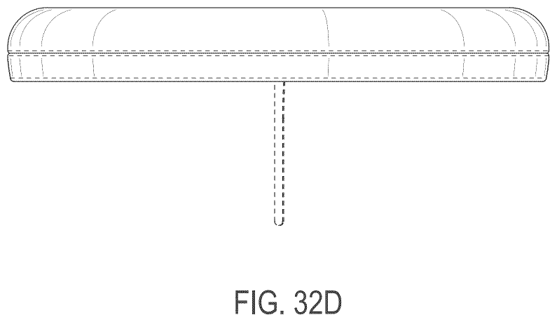

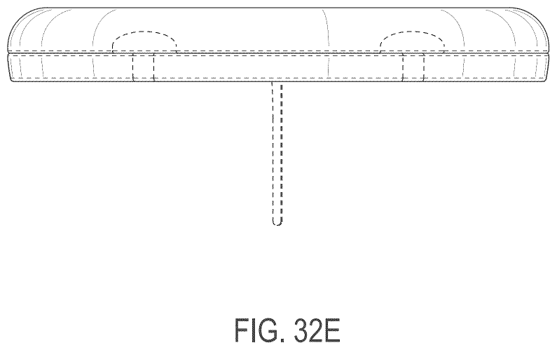

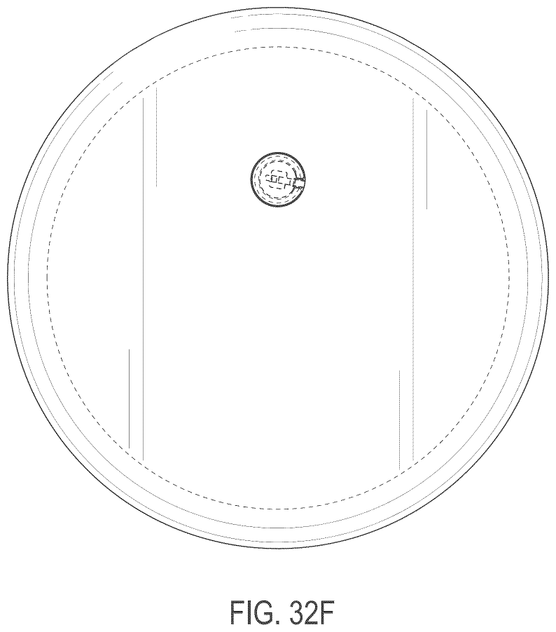

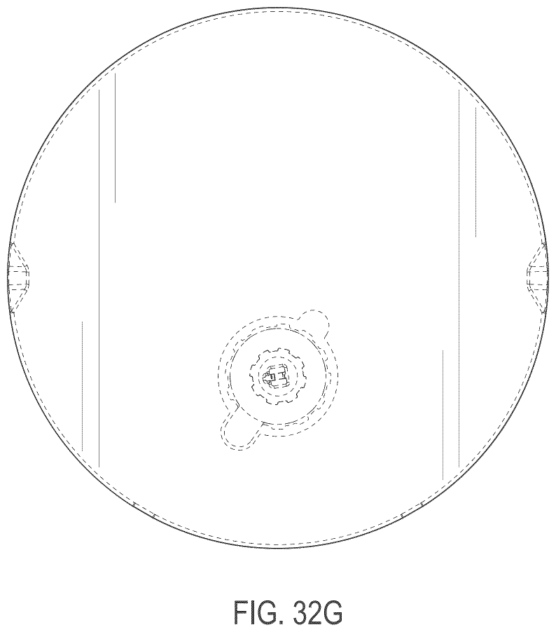

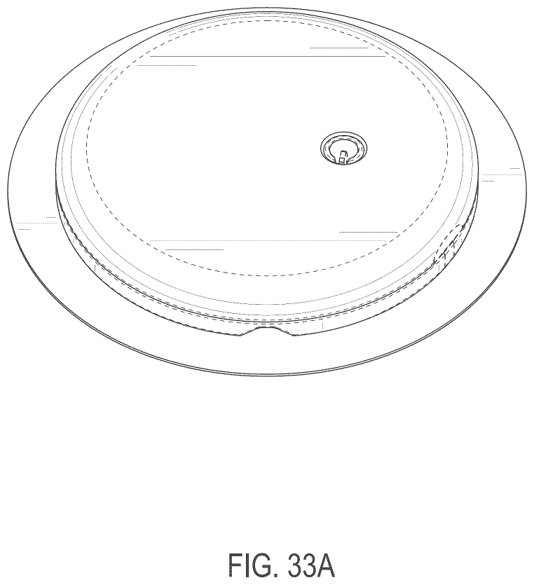

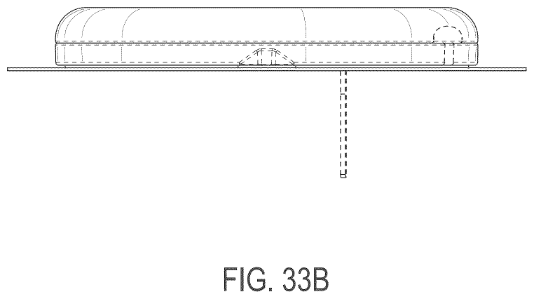

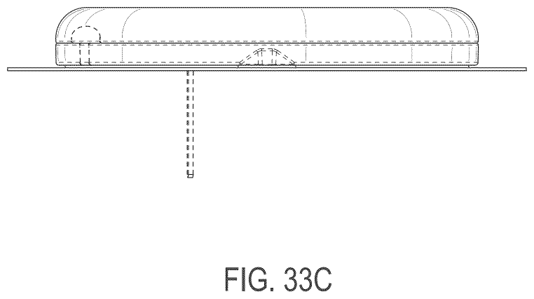

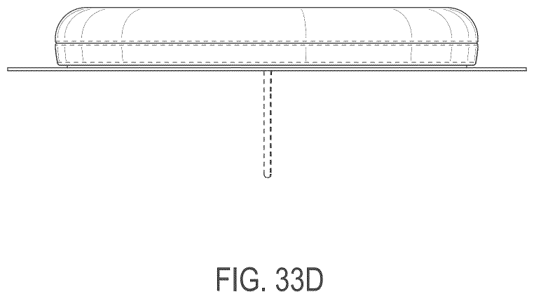

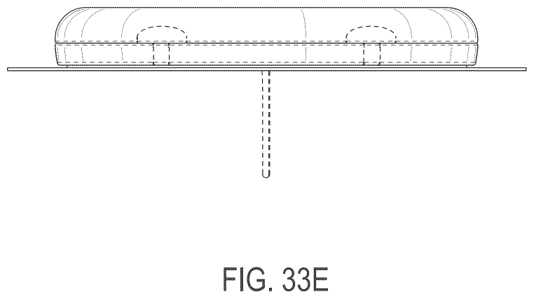

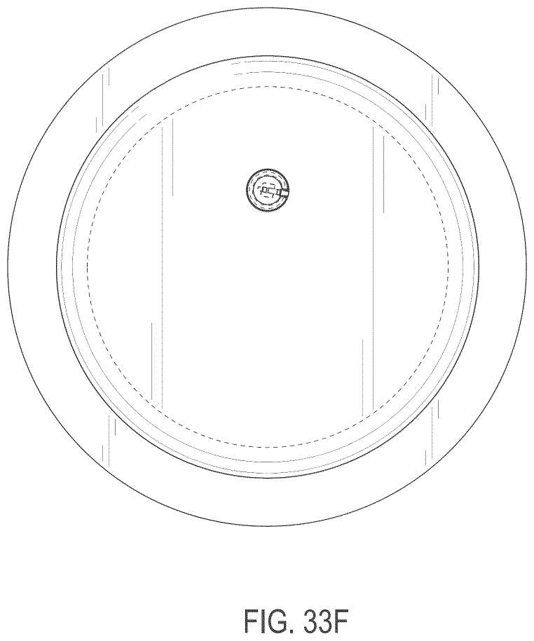

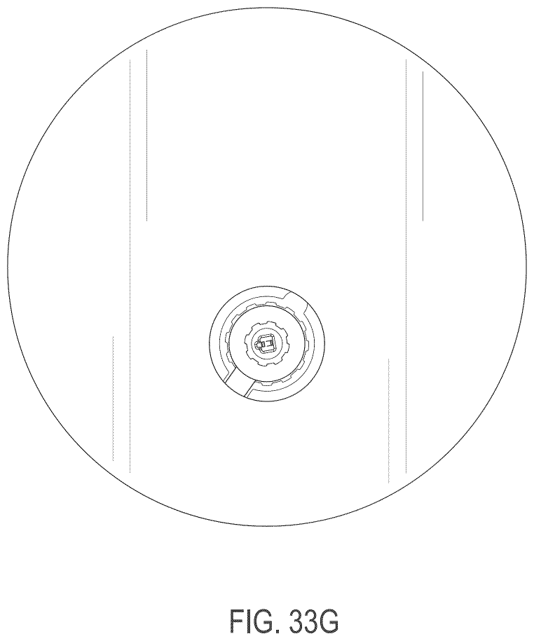

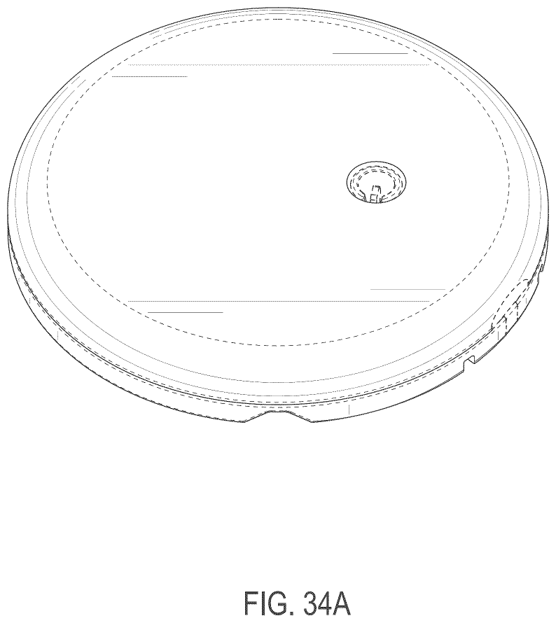

FIGS. 32A-32G depict a first embodiment of a sensor control device for sterilization with, e.g., an FEB, and without an adhesive patch. FIGS. 33A-33G depict a second embodiment of the sensor control device for sterilization with, e.g., an FEB, along with an adhesive patch.

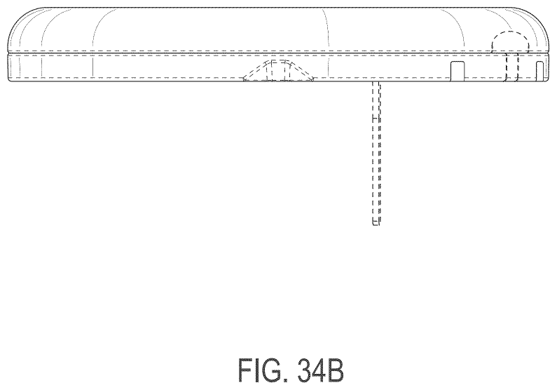

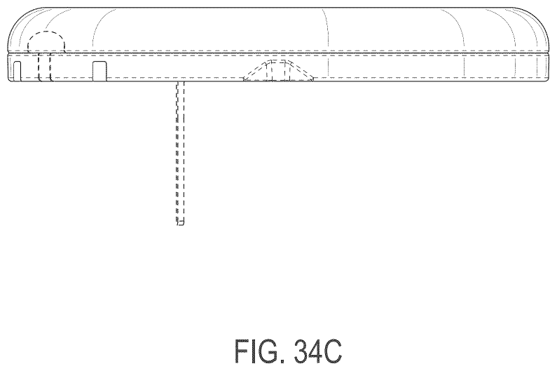

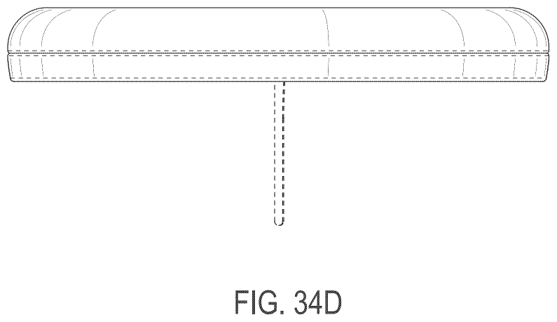

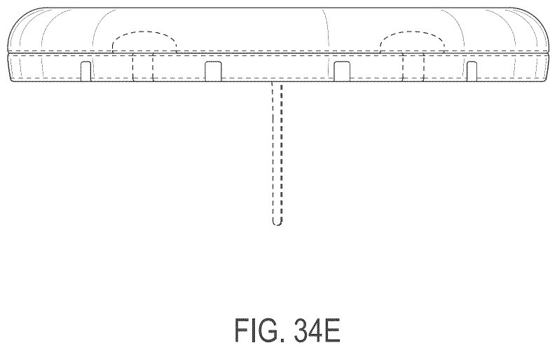

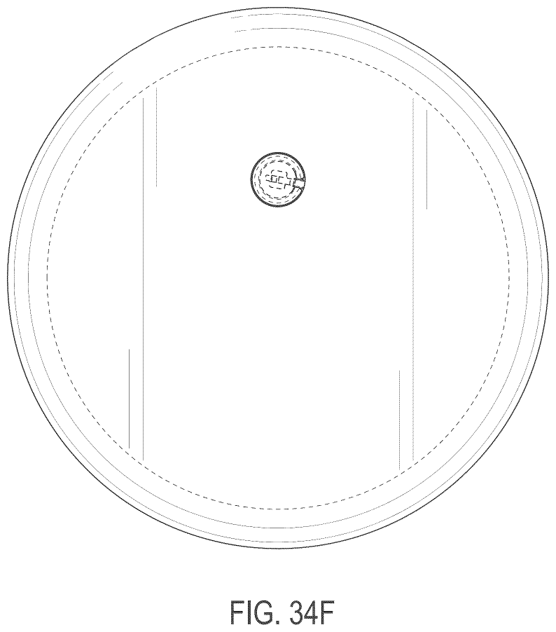

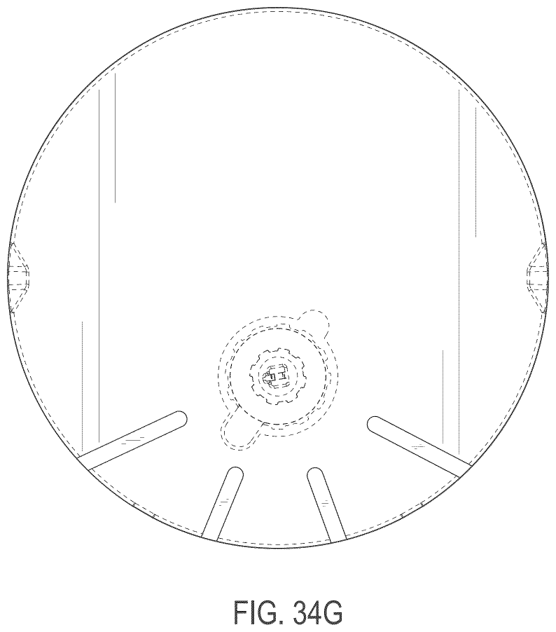

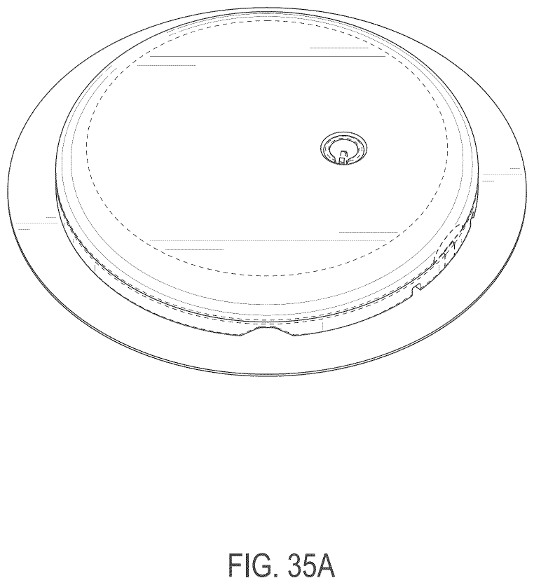

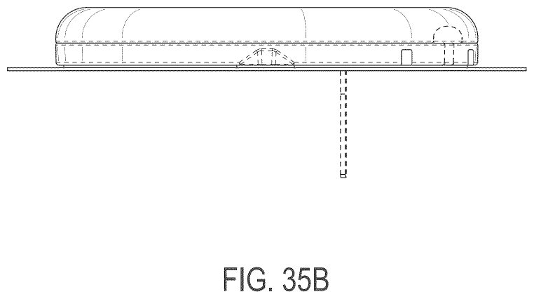

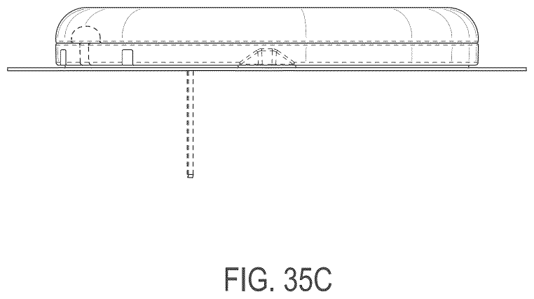

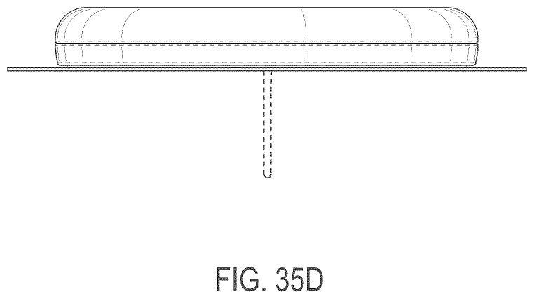

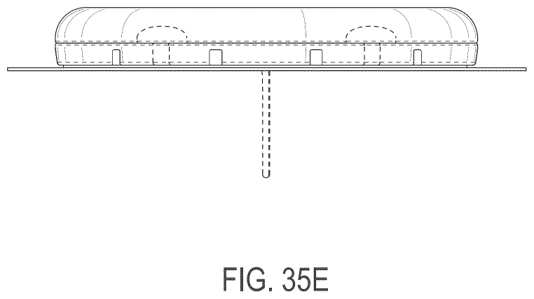

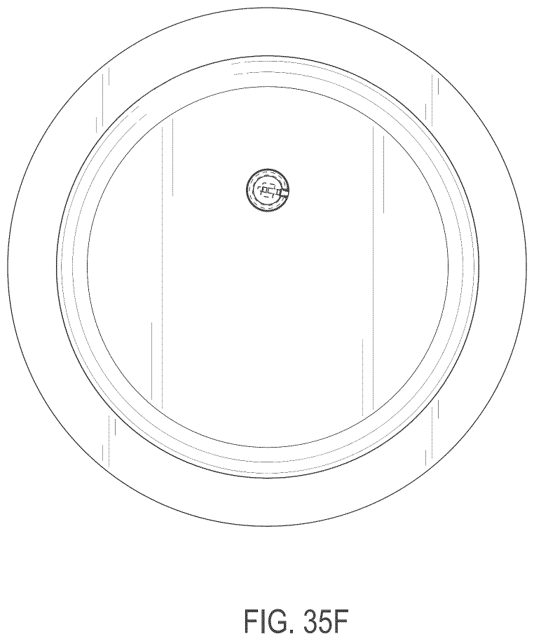

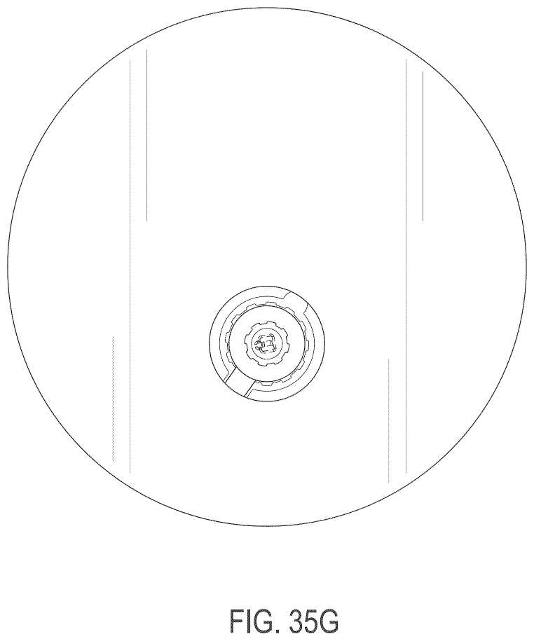

FIGS. 34A-34G depict a third embodiment of a sensor control device having bottom surface grooves and for sterilization with, e.g., an FEB, but without an adhesive patch. FIGS. 35A-35G depict a fourth embodiment of the sensor control device having bottom surface grooves and for sterilization with, e.g., an FEB, along with an adhesive patch.

For all of the embodiments shown and described in FIGS. 20A-35G, solid lines can be alternatively depicted as broken lines, which form no part of the design. For all of the embodiments of sensor control devices described in FIGS. 22A-29G and 32A-35G, the adhesive patch, if shown in solid line, can alternatively be shown in broken line, and the adhesive patch, if not shown, can be shown in broken or solid line.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

In many example embodiments, a method for applying a medical device to a subject using an applicator is provided, the method including: positioning a distal end of the applicator on a skin surface of the subject, where at least a portion of the distal end includes a compressible material; applying a force on the applicator to cause the medical device to advance from a first position within the applicator to a second position adjacent to the skin surface, and to cause the distal end of the applicator to stretch and flatten a portion of the skin surface adjacent to the applicator; and applying the medical device to the stretched and flattened portion of the skin surface.

In these method embodiments, applying a force on the applicator can further include displacing the at least the compressible portion of the distal end of the applicator in a radially outward direction. Displacing the at least the compressible portion of the distal end of the applicator can further include creating radially outward forces on the portion of the skin surface adjacent to the applicator.

In these method embodiments, applying the medical device to the stretched and flattened portion of the skin surface can further include placing an adhesive surface on the skin surface.

In these method embodiments, applying the medical device to the stretched and flattened portion of the skin surface can further include positioning at least a portion of an analyte sensor under the skin surface. The analyte sensor can be an in vivo analyte sensor configured to measure an analyte level in a bodily fluid of the subject.

In these method embodiments, the at least the compressible portion of the distal end of the applicator can be biased in a radially inward direction. Alternatively, the at least the compressible portion of the distal end of the applicator can be biased in a radially outward direction.

In these method embodiments, the at least the compressible portion of the distal end can be in an unloaded state in the first position, and the at least the compressible portion of the distal end can be in a loaded state in the second position.

In these method embodiments, the at least the compressible portion of the distal end of the applicator can include one or more of an elastomeric material, metal, plastic, or composite legs or springs, or a combination thereof.

In these method embodiments, a cross-section of the at least the compressible portion of the distal end of the applicator can include a continuous ring or a non-continuous shape.

In these method embodiments, the distal end of the applicator can be configured to be detached from the applicator.

In many example embodiments, an apparatus is provided including: a medical device; and an applicator including a distal end configured to be positioned on a skin surface of a subject, where at least a portion of the distal end includes a compressible material, where, in response to an application of force to the applicator: the medical device can be configured to advance from a first position within the applicator to a second position adjacent to the skin, the distal end of the applicator can be configured to stretch and flatten a portion of the skin surface adjacent to the applicator, and the medical device can be further configured to be applied to the stretched and flattened portion of the skin surface.

In these apparatus embodiments, the at least the compressible portion of the distal end of the applicator can be configured to displace in a radially outward direction in response to the application of force to the applicator. The at least the compressible portion of the distal end of the applicator can be further configured to create radially outward forces on the portion of the skin surface adjacent to the applicator.

In these apparatus embodiments, the medical device can include an adhesive surface that can be configured to interface with the skin surface.

In these apparatus embodiments, the medical device can include an analyte sensor at least a portion of which can be configured to be positioned under the skin surface. The analyte sensor can be an in vivo analyte sensor configured to measure an analyte level in a bodily fluid of the subject.

In these apparatus embodiments, the at least the compressible portion of the distal end of the applicator can be biased in a radially inward direction. Alternatively, the at least the compressible portion of the distal end of the applicator can be biased in a radially outward direction.

In these apparatus embodiments, the at least the compressible portion of the distal end can be in an unloaded state in the first position, and where the at least the compressible portion of the distal end can be in a loaded state in the second position.

In these apparatus embodiments, the at least the compressible portion of the distal end of the applicator can include one or more of an elastomeric material, metal, plastic, or composite legs or springs, or a combination thereof.

In these apparatus embodiments, a cross-section of the at least the compressible portion of the distal end of the applicator can include a continuous ring or a non-continuous shape.

In these apparatus embodiments, the distal end of the applicator can be configured to be detached from the applicator.

In many embodiments, an assembly for use in an applicator is provided, the assembly including: a sharp module including a sharp portion and a hub portion, where the sharp portion can include a sharp shaft, a sharp proximal end coupled to a distal end of the hub portion, and a sharp distal tip configured to penetrate a skin surface of a subject, where the sharp module can further include a plastic material.

In these assembly embodiments, the sharp shaft can include one or more filleted edges.

In these assembly embodiments, the sharp module can further include a thermoplastic material.

In these assembly embodiments, the sharp module can further include a polyether ether ketone material.

In these assembly embodiments, the sharp shaft can include an alignment ledge configured to prevent rotational movement along a vertical axis during an insertion process. The alignment ledge can be positioned along a proximal portion of the sharp shaft.

In these assembly embodiments, the assembly can further include an analyte sensor, where the analyte sensor can be an in vivo analyte sensor configured to measure an analyte level in a bodily fluid of the subject. A distal end of the analyte sensor can be in a proximal position relative to the sharp distal tip. A distal end of the analyte sensor and the sharp distal tip can be co-localized. At least a portion of the analyte sensor can be positioned within a sensor channel of the sharp shaft.

In these assembly embodiments, the sharp module can further include a liquid crystal polymer material.

In these assembly embodiments, the assembly can further include a lubricant disposed on an external surface of the sharp module.

In these assembly embodiments, the plastic material can include a lubricant.

In these assembly embodiments, the assembly can further include a sensor channel, where at least a portion of the sensor channel can be disposed in a distal portion of the sharp shaft. The sensor channel can extend from the proximal portion of the sharp shaft to the distal portion of the sharp shaft. The sensor channel can be configured such that it does not extend beyond the distal portion of the sharp shaft. The proximal portion of the sharp shaft can be hollow. The proximal portion of the sharp shaft can be solid. A wall thickness of at least a portion of the proximal portion of the sharp shaft can be greater than a wall thickness of the distal portion of the sharp shaft.

In these assembly embodiments, the assembly can further include one or more rib structures adjacent to the hub portion, where the one or more rib structures can be configured to reduce a compressive load around the hub portion.

In many embodiments, a method of preparing an analyte monitoring system is provided, the method including: loading a sensor control device into a sensor applicator, the sensor control device including: an electronics housing; a printed circuit board positioned within the electronics housing and including a processing circuitry; an analyte sensor extending from a bottom of the electronics housing; and a sharp module including a plastic material and removably coupled to the electronics housing, where the sharp module includes a sharp, and where the sharp extends through the electronics housing and receives a portion of the analyte sensor extending from the bottom of the electronics housing; securing a cap to the sensor applicator and thereby providing a barrier that seals the sensor control device within the sensor applicator; and sterilizing the analyte sensor and the sharp with radiation while the sensor control device can be positioned within the sensor applicator.

In these method embodiments, the sensor control device can further include at least one shield positioned within the electronics housing, and where the method can further include shielding the processing circuitry with the at least one shield from the radiation during the sterilization. The at least one shield can include a magnet, and where shielding the processing circuitry with the at least one shield can include: generating a static magnetic field with the magnet; and diverting the radiation away from the processing circuitry with the static magnetic field. Sterilizing the analyte sensor and the sharp with radiation can further include using a non-focused electron beam to sterilize the analyte sensor and the sharp.

In these method embodiments, the analyte sensor can be an in vivo analyte sensor configured to measure an analyte level in a bodily fluid located in the subject.

In these method embodiments, the sharp module can further include a thermoplastic material.

In these method embodiments, the sharp module can further include a polyether ether ketone material.

In these method embodiments, sterilizing the analyte sensor and the sharp can further include focusing an electron beam on the analyte sensor and the sharp.

In many embodiments, an assembly for use in an applicator is provided, the assembly including: a sharp module including a sharp portion and a hub portion, where the sharp portion can include a sharp shaft, a sharp proximal end coupled to a distal end of the hub portion, and a sharp distal tip configured to penetrate a skin surface of a subject, where the sharp portion can further include a metal material and can be formed through a coining process.

In these assembly embodiments, the sharp portion can further include a stainless steel material.

In these assembly embodiments, the sharp portion includes no sharp edges.

In these assembly embodiments, the sharp portion can include one or more rounded edges.

In these assembly embodiments, the sharp shaft can include one or more rounded edges.

In these assembly embodiments, the sharp shaft and the sharp distal tip can include one or more rounded edges.

In these assembly embodiments, the assembly can further include an analyte sensor, where the analyte sensor can be an in vivo analyte sensor configured to measure an analyte level in a bodily fluid of the subject. A distal end of the analyte sensor can be in a proximal position relative to the sharp distal tip. A distal end of the analyte sensor and the sharp distal tip can be co-localized. At least a portion of the analyte sensor can be positioned within a sensor channel of the sharp shaft.

In many embodiments, a method of maintaining structural integrity of a sensor control unit including an analyte sensor and a sensor module is provided, the method including: positioning a distal sensor portion of the analyte sensor beneath a skin surface and in contact with a bodily fluid, where the analyte sensor can include a proximal sensor portion coupled to the sensor module, and where the proximal sensor portion includes a hook feature adjacent to a catch feature of the sensor module; receiving one or more forces in a proximal direction along a longitudinal axis of the analyte sensor; and causing the hook feature to engage the catch feature and prevent displacement of the analyte sensor in the proximal direction along the longitudinal axis.

In these method embodiments, the method can further include loading the analyte sensor into the sensor module by displacing the proximal sensor portion in a lateral direction to bring the hook feature in proximity to the catch feature of the sensor module. Displacing the proximal sensor portion in a lateral direction can include causing the proximal sensor portion to move into a clearance area of the sensor module.

In these method embodiments, the one or more forces can be generated by a sharp retraction process.

In these method embodiments, the one or more forces can be generated by a physiological reaction to the analyte sensor.

In these method embodiments, the analyte sensor can be an in vivo analyte sensor configured to measure an analyte level in the bodily fluid of the subject.

In many embodiments, a sensor control unit is provided, the sensor control unit including: a sensor module including a catch feature; an analyte sensor including a distal sensor portion and a proximal sensor portion, where the distal sensor portion can be configured to be positioned beneath a skin surface and in contact with a bodily fluid, and where the proximal sensor portion can be coupled to the sensor module and can include a hook feature adjacent to the catch feature, where the hook feature can be configured to engage the catch feature and prevent displacement of the analyte sensor caused by one or more forces received by the analyte sensor and in a proximal direction along a longitudinal axis of the analyte sensor.

In these sensor control unit embodiments, the sensor module can be configured to receive the analyte sensor by displacing the proximal sensor portion in a lateral direction and bringing the hook feature in proximity to the catch feature of the sensor module. The sensor module can further include a clearance area configured to receive the proximal sensor portion as the proximal sensor portion can be displaced in a lateral direction.

In these sensor control unit embodiments, the one or more forces can be generated by a sharp retraction process.

In these sensor control unit embodiments, the one or more forces can be generated by a physiological reaction to the analyte sensor.

In these sensor control unit embodiments, the analyte sensor can be an in vivo analyte sensor configured to measure an analyte level in the bodily fluid of the subject.

In many embodiments, a method of inserting an analyte sensor into a subject using an applicator is provided, the method including: positioning a distal end of the applicator on a skin surface, where the applicator can include a drive spring, a retraction spring, a sensor carrier, a sharp carrier, and the analyte sensor; applying a first force to the applicator to cause the drive spring to displace the sensor carrier and the sharp carrier from a first position within the applicator in spaced relation with a skin surface to a second position adjacent to the skin surface, and to position a sharp of the sharp carrier and a portion of the analyte sensor under the skin surface and in contact with a bodily fluid of the subject; and applying a second force to the applicator to cause the retraction spring to displace the sharp carrier from the second position to a third position within the applicator, and to withdraw the sharp from the skin surface.

In these method embodiments, applying the first force can include applying a force in a distal direction, and where applying the second force can include applying a force in a proximal direction.

In these method embodiments, the applicator can further include a firing pin and a sheath, and where applying the first force to the applicator further causes the firing pin to disengage one or more sheath tabs of the sheath from one or more sensor carrier latches of the sensor carrier and to cause the drive spring to expand. The drive spring can be in a preloaded state prior to applying the first force, and where disengaging the one or more sheath tabs causes the drive spring to expand in a distal direction. Applying the first force to the applicator increases a load on the drive spring prior to causing the firing pin to disengage the one or more sheath tabs. The drive spring can be in a preloaded state prior to applying the first force, and where the drive spring can include a first end coupled to the firing pin and a second end coupled to the sensor carrier.

In these method embodiments, the applicator can further include a sensor control unit coupled with the sensor carrier, and where a distal portion of the sensor control unit can be in contact with the skin surface in the second position. Displacing the sensor carrier and the sharp carrier from the first position to the second position can include one or more sensor carrier tabs of the sensor carrier traveling in a distal direction along one or more sheath rails of the sheath. One or more sensor carrier bumpers of the sensor carrier can be biased against an internal surface of the sheath while the sensor carrier and the sharp carrier can be displaced from the first position to the second position.

In these method embodiments, applying the second force further causes a plurality of sensor carrier lock arms of the sensor carrier to disengage from the sharp carrier and to cause the retraction spring to expand. Disengaging the plurality of sensor carrier lock arms from the sharp carrier can include positioning the plurality of sensor carrier lock arms into a plurality of sheath notches of the sheath. Each of the plurality of sensor carrier locks arms can be biased in a radially outward direction, and where the sheath notches can be configured to allow the plurality of sensor carrier lock arms to expand in a radially outward direction. The retraction spring can be in a preloaded state prior to applying the second force, and where disengaging the plurality of sensor carrier lock arms causes the retraction spring to expand in a proximal direction.

In these method embodiments, the retraction spring can be in a preloaded state prior to applying the second force, and where the retraction spring can include a first end coupled to the sharp carrier and a second end coupled to the sensor carrier.

In these method embodiments, applying the second force further causes the drive spring to displace the sensor carrier to a bottom portion of the applicator.

In these method embodiments, the analyte sensor can be an in vivo analyte sensor configured to measure an analyte level in the bodily fluid of the subject.

In many embodiments, an applicator for inserting an analyte sensor into a subject is provided, the applicator including: a drive spring; a retraction spring; a sensor carrier; a sharp carrier coupled to a sharp; and the analyte sensor; where the drive spring can be configured to displace the sensor carrier and the sharp carrier from a first position within the applicator in spaced relation with a skin surface to a second position adjacent to the skin surface upon an application of a first force to the applicator, and where the sharp and a portion of the analyte sensor can be positioned under the skin surface and in contact with a bodily fluid of the subject at the second position, and where the retraction spring can be configured to displace the sharp carrier from the second position to a third position within the applicator and to withdraw the sharp from the skin surface upon an application of a second force to the applicator.

In these applicator embodiments, the application of the first force can include an application of a force in a distal direction, and where the application of the second force can include an application of a force in a proximal direction.

In these applicator embodiments, the applicator can further include a firing pin and a sheath, where the firing pin can be configured to, upon application of the first force, disengage one or more sheath tabs of the sheath from one or more sensor carrier latches of the sensor carrier and to cause the drive spring to expand. The drive spring can be in a preloaded state prior to the application of the first force, and where the drive spring can be configured to expand in a distal direction in response to the one or more sheath tabs disengaging from the one or more sensor carrier latches. The drive spring can be configured to receive an increased load prior to the firing pin disengaging the one or more sheath tabs. The drive spring can be in a preloaded state prior to the application of the first force, and where the drive spring can include a first end coupled to the firing pin and a second end coupled to the sensor carrier.

In these applicator embodiments, the applicator can further include a sensor control unit coupled with the sensor carrier, where a distal portion of the sensor control unit can be configured to contact the skin surface in the second position.

In these applicator embodiments, the applicator can further include one or more sensor carrier tabs of the sensor carrier configured to travel in a distal direction along one or more sheath rails of the sheath between the first position and the second position.

In these applicator embodiments, the applicator can further include one or more sensor carrier bumpers of the sensor carrier configured to bias against an internal surface of the sheath between the first position and the second position.

In these applicator embodiments, the applicator can further include a plurality of sensor carrier lock arms of the sensor carrier, where the sensor carrier lock arms can be configured to disengage from the sharp carrier and cause the retraction spring to expand in response to the application of the second force. The applicator can further include a plurality of sheath notches of the sheath, where the plurality of sheath notches can be configured to receive the plurality of sensor carrier lock arms and to cause the sensor carrier lock arms to disengage from the sharp carrier. Each of the plurality of sensor carrier locks arms can be biased in a radially outward direction, and where the sheath notches can be configured to allow the plurality of sensor carrier lock arms to expand in a radially outward direction. The retraction spring can be in a preloaded state prior to the application of the second force, and where the retraction spring can be configured to expand in a proximal direction when the plurality of sensor carrier lock arms disengages from the sharp carrier.

In these applicator embodiments, the retraction spring can be in a preloaded state prior to the application of the second force, and where the retraction spring can include a first end coupled to the sharp carrier and a second end coupled to the sensor carrier.

In these applicator embodiments, the drive spring can be further configured to displace the sensor carrier to a bottom portion of the applicator in response to the application of the second force.

In these applicator embodiments, the analyte sensor can be an in vivo analyte sensor configured to measure an analyte level in the bodily fluid of the subject.

In many embodiments, an assembly for use in an applicator is provided, the assembly including: a sharp module including a sharp portion and a hub portion, where the sharp portion can include a sharp shaft, a sharp proximal end coupled to the hub portion, and a sharp distal tip configured to penetrate a skin surface of a subject, where the sharp shaft includes a sensor channel configured to receive at least a portion of an analyte sensor, where the sensor channel can be in a spaced relation to the sharp distal tip, and where the sharp distal tip includes an offset tip portion configured to create an opening in the skin surface.

In these assembly embodiments, the sharp module can further include a stainless steel material.

In these assembly embodiments, the sharp module can further include a plastic material.

In these assembly embodiments, where the offset tip portion can be further configured to prevent damage to a sensor tip portion of the analyte sensor during a sensor insertion process.

In these assembly embodiments, a cross-sectional area of the offset tip portion can be less than a cross-sectional area of the sharp shaft.

In these assembly embodiments, the offset tip portion can include a separate element coupled to the sharp shaft.

In these assembly embodiments, the sensor channel can include one or more sidewalls of the sharp shaft. The offset tip portion can be formed from a portion of the one or more sidewalls of the sharp shaft. The sensor channel can include a first sidewall and a second sidewall, where the offset tip portion can be formed from a terminus of the first sidewall of the sharp shaft, and where a terminus of the second sidewall can be proximal to the terminus of the first sidewall.

In many embodiments, a method of manufacturing an analyte monitoring system is provided, including: sterilizing a sensor sub-assembly including a sensor and a sharp; assembling the sterilized sensor sub-assembly into a sensor control device; assembling the sensor control device into an applicator; and packaging the applicator, having the sensor control device therein, for distribution.

In these method embodiments, the sensor control device can be as shown or substantially as shown in any of FIGS. 20A-21G.

In these method embodiments, the applicator can be as shown or substantially as shown in any of FIGS. 22A-29G.

In many embodiments, a method of manufacturing an analyte monitoring system is provided, the method including: assembling a sensor control device including a sensor and a sharp; assembling the sensor control device into an applicator; sterilizing the applicator, having the sensor control device therein, with a focused electron beam; and packaging the applicator, having the sensor control device therein, for distribution.

In these method embodiments, the sensor control device can be as shown or substantially as shown in any of FIGS. 30A-31G.

In these method embodiments, the applicator can be as shown or substantially as shown in any of FIGS. 32A-35G.

### Example Embodiments of Environmentally Conscious Packaging and Components

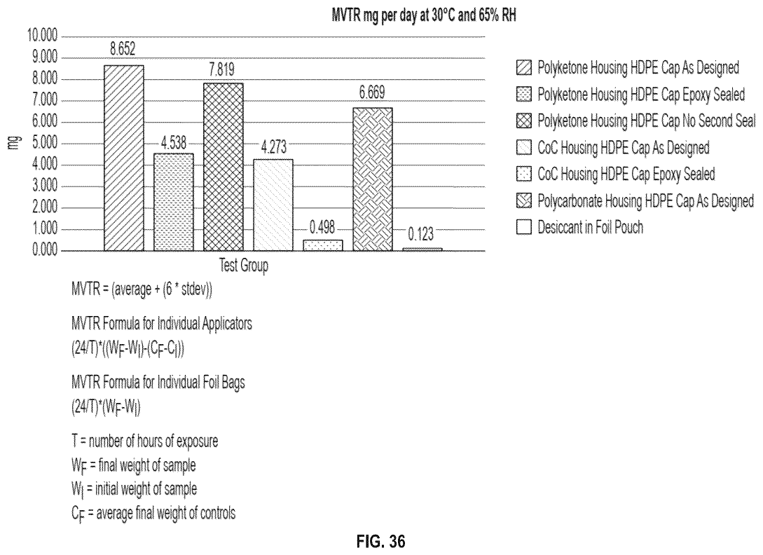

According to embodiments of the present disclosure, analyte monitoring systems that incorporate a two-piece or a one-piece architecture may be shipped to a user in a sealed package. More particularly, in embodiments employing a two-piece architecture, applicator 150 and sensor container or tray 810 can be shipped in a single sealed package. Alternatively, applicator 150 can and sensor container or tray 810 can be shipped in separate sealed packages. In contrast, in embodiments employing a one-piece architecture, one-piece applicator 5150 can be shipped in a single sealed package. According to embodiments of the present disclosure, sealed package can include sealed foil bags or any other sealed package known to a person of ordinary skill in the art. The sealed package described herein can be designed to maintain a low moisture vapor transition rate (MVTR), thereby enabling stable shelf life for one-piece and two-piece analyte monitoring systems. For example, as shown in the chart depicted in FIG. 36, the MVTR was tested at 30 C and 65% relative humidity for a number of different materials and seals.

According to embodiments of the present disclosure, sealed package may be resealable. For example, sealed packaging can include resealing mechanism such as zip-type interlocking closure, or any other method or system known to a person of ordinary skill in the art.

Additionally, sealed package may include a pre-paid, pre-printed return shipping label allowing users to return used applicators, containers, and/or sensor control devices for recycling or sharps for disposal. Moreover, sealed package described herein may prove advantageous in eliminating component parts and various fabrication process steps. For example, by carefully planning humidity control during manufacturing, sealed package described herein may either eliminate the need for a desiccant or allow use of a smaller off-the-shelf desiccant within the sealed package. Furthermore, pressure decay leak testing may no longer be required during the manufacturing processes. For example, pressure decay testing is conducted during manufacturing once applicator has been assembled and packaged. As such, housing and cap are designed using material that can achieve a proper seal between components to ensure the product meets its intended shelf life. However, if a foil sealed bag is utilized, stringent pressure decay test of different components is no longer required.

According to embodiments of the present disclosure, any of the applicator embodiments described herein, as well as any of the components thereof, including but not limited to the housing, sheath, sharp carrier, electronics carrier, firing pin, sharp hub, sensor module embodiments, actuator, and sensor container or tray may be made of a variety of rigid materials. In some embodiments, for example, the components may be made of an engineered thermoplastic, such as acetal or polyoxymethylene. Use of a single material for the construction of the various components of the applicator embodiments described herein may be advantageous in improving recyclability, lubricity, and tight tolerance control. Specifically, acetal can be used to provide lubricity (i.e., low friction) between parts which move relative to each other, for example, sheath and housing, sharp carrier and housing. As such, reducing friction can help provide sufficient force to achieve successful sensor insertion. Use of acetal can additionally reduce the need for pressure decay testing during manufacturing. In other embodiments, for example, other materials having the same or similar properties to acetal, such as polybutylene terephthalate (PBT), can be used for any or all of the aforementioned components. Additionally, use of a sealable package reduces the need for tight component tolerance control generally required to achieve a proper seal between applicator housing to cap, therefore allowing a single material to be used for manufacture. Tighter tolerance parts generally require tightly controlled tooling and processes, thereby increasing manufacturing costs for parts. Use of a single material can therefore reduce manufacturing costs. For example, after separation of any metallic components such as, drive spring, battery, and retraction spring, using a magnet, all remaining components made form the same material may be easily recycled.

Exemplary embodiments and features are set out in the following numbered clauses:

1. An assembly for delivery of an analyte sensor comprising:
a reusable applicator configured to deliver a first analyte sensor, the reusable applicator having a proximal portion and a distal portion and including:
   a housing;
   a sensor carrier configured to releasably receive the first analyte sensor;
   a sharp carrier configured to releasably receive a sharp module; and
   an actuator moveable relative to the housing, the actuator having:
      a first position with the sensor carrier and the sharp carrier at the proximal portion of the reusable applicator,
      a second position with the sensor carrier and the sharp carrier at the distal portion of the reusable applicator for delivery of the first analyte sensor from the reusable applicator, and
      a third position with the sensor carrier at the distal portion of the reusable applicator and the sharp carrier at the proximal portion of the reusable applicator after delivery of the first analyte sensor,
      wherein the first position, the second position, and the third position are different, and wherein the actuator is configured to be returned from the third position to the first position for delivery of another analyte sensor.

2. The assembly for delivery of an analyte sensor of clause 1, wherein the reusable applicator further includes a drive spring to move the sensor carrier and the sharp carrier from the proximal portion to the distal portion and a retraction spring to move the actuator to the third position.

3. The assembly for delivery of an analyte sensor of clause 2, wherein the drive spring is actuated by movement of the actuator from the first position to the second position.

4. The assembly for delivery of an analyte sensor of clause 2 or 3, wherein the retraction spring is actuated by movement of the sensor carrier from the proximal position to the distal position of the reusable applicator.

5. The assembly for delivery of an analyte sensor of any of clauses 1 to 4, wherein the reusable applicator further includes a latch to hold the sensor carrier at the distal portion of the reusable applicator when the actuator is moved from the second position toward the third position.

6. The assembly for delivery of an analyte sensor of any of clauses 1 to 5, wherein with the actuator in the third position, the sharp carrier is accessible from the proximal portion of the reusable applicator to release the sharp module.

7. The assembly for delivery of an analyte sensor of any of clauses 1 to 6, wherein the actuator further comprises a visual indicator of a position of the actuator.

8. The assembly for delivery of an analyte sensor of clause 7, wherein the actuator includes a button configured to extend a first predetermined length relative the housing in the first position, a second predetermined length relative the housing in the second position, and a third predetermined length relative the housing in the third position, and wherein the third predetermined length is greater than the first predetermined length and the first predetermined length is greater than the second predetermined length.

9. The assembly for delivery of an analyte sensor of clause 8, wherein the button configured to be opened for removal of the sharp module.

10. The assembly for delivery of an analyte sensor of any of clauses 1 to 9, wherein the reusable applicator assembly is made of a recyclable material.

11. The assembly for delivery of an analyte sensor of clause 10, wherein the reusable applicator comprises acetal.

12. The assembly for delivery of an analyte sensor of any of clauses 1 to 11, further comprising a sealable container to package the reusable applicator assembly.

13. The assembly for delivery of an analyte sensor of clause 12, wherein the sealable container has a low moisture vapor transition rate.

14. The assembly for delivery of an analyte sensor of clause 13, wherein the sealable container is configured to eliminate the need for a desiccant.

15. The assembly for delivery of an analyte sensor of any of clauses 1 to 14, further comprising an applicator cap sealingly coupled to the housing with a gasketless seal.

16. A method of using an assembly for delivery of an analyte sensors, comprising:
providing a reusable applicator having a proximal portion and a distal portion, the reusable applicator including a housing, a sensor carrier having a first analyte sensor releasably received therein, a sharp carrier having a sharp module releasably received therein, and an actuator moveable relative to the housing;
moving the actuator of the reusable applicator assembly from a first position to a second position to move the sensor carrier and the sharp carrier from the proximal portion of the reusable applicator toward the distal portion of the reusable applicator to deliver the analyte sensor from the sensor carrier;
moving the sharp carrier from the distal portion of the reusable applicator toward the proximal portion of the reusable applicator and moving the actuator of the reusable applicator assembly to a third position after delivery of the first analyte sensor; and
returning the actuator from the third position to the first position for receipt of another analyte sensor for delivery;
wherein the first position, the second position, and the third position are different.

17. The method of using an assembly for delivery of an analyte sensor of clause 16, wherein the reusable applicator further includes a drive spring to move the sensor carrier and the sharp carrier from the proximal portion to the distal portion.

18. The method of using an assembly for delivery of an analyte sensor of clause 16 or 17, wherein the reusable applicator further includes a retraction spring to move the actuator to the third position.

19. The method of using an assembly for delivery of an analyte sensor of clause 17 or 18, wherein after returning the actuator from the third position to the first position the method further includes:
reloading, using the actuator of the reusable applicator assembly, the retraction spring by moving the sharp carrier from the proximal portion of the reusable applicator to the distal portion of the reusable applicator; and
reloading the drive spring by moving the sensor carrier and the sharp carrier from the distal portion of the reusable applicator to the proximal portion of the reusable applicator.

20. The method of using an assembly for delivery of an analyte sensor of any of clauses 16 to 19, wherein the reusable applicator further includes a latch to hold the sensor carrier at the distal portion of the reusable applicator when the actuator moves from the second position toward the third position.

21. The method of using an assembly for delivery of an analyte sensor of any of clauses 16 to 20, further comprising accessing the sharp carrier from the proximal portion of the reusable applicator for releasing the sharp module.

22. The method of using an assembly for delivery of an analyte sensor of any of clauses 16 to 21, wherein the reusable applicator includes a visual indicator of a position of the actuator.

23. The method of using an assembly for delivery of an analyte sensor of any of clauses 16 to 22, wherein the actuator includes a button, and the method further comprises opening the button to access and remove the first sharp module when the actuator is in the third position.

24 An assembly comprising:
a reusable applicator configured to insert at least a portion of an analyte sensor under a skin surface and in contact with a bodily fluid, the reusable applicator comprising:
   a housing;
   an actuator configured to move in a distal direction relative to the housing;
   a sharp carrier releasably coupled with a sharp module;
   a reusable applicator base releasably engaged with a disposable sensor carrier, the disposable sensor carrier configured to releasably retain a sensor control device; and
the sensor control device comprising the analyte sensor,
wherein the sensor control device is configured to advance in the distal direction from a first position within the reusable applicator to a second position adjacent to the skin surface after application of a first force on the actuator, and
wherein the reusable applicator is further configured to eject the disposable sensor carrier and the sharp module therefrom in response to application of a second force on the actuator.

25. The assembly of clause 24, further comprising a drive spring comprising a first end in contact with a firing pin, wherein the application of the first force on the actuator causes the drive spring to expand.

26 The assembly of clause 25, further comprising a retraction spring disposed within the sharp carrier.

27. The assembly of clause 26, wherein the reusable applicator base comprises one or more carrier lock arms configured to engage the sharp carrier.

28. The assembly of clause 27, wherein expansion of the drive spring causes the reusable applicator base, the retraction spring, the sharp carrier, the sharp module, the disposable sensor carrier, and the sensor control device to advance in the distal direction towards the skin surface.

29. The assembly of clause 28, wherein the one or more carrier lock arms are configured to disengage from the sharp carrier as the sensor control device is advanced from the first position to the second position, and wherein the retraction spring is configured to expand after the one or more carrier lock arms are disengaged from the sharp carrier.

30. The assembly of any of clauses 24 to 29, wherein the sharp module comprises a sharp hub configured to engage with the sharp carrier.

31. The assembly of clause 30, wherein the sharp module further comprises a sharp.

32. The assembly of any of clauses 24 to 31, wherein the disposable sensor carrier comprises one or more latches configured to couple with one or more corresponding ledges of the reusable applicator base.

33. The assembly of any of clauses 24 to 32, wherein the actuator is at a first height relative to the housing before the application of the first force, wherein the actuator is configured to return to a second height relative to the housing after the application of the first force, and wherein the second height is greater than the first height.

34. The assembly of any of clauses 24 to 33, wherein the application of the second force on the actuator causes a distal portion of the actuator to contact the sharp carrier.

35. The assembly of clause 34, wherein the sharp carrier comprises one or more sharp carrier retention arms, and wherein the contact by the distal portion of the actuator with the sharp carrier causes the one or more sharp carrier retention arms to spread apart and disengage from the sharp module.

36. The assembly of clause 35, wherein the distal portion of the actuator is configured to eject the sharp module from the sharp carrier.

37. The assembly of any of clauses 24 to 36, wherein the application of the second force on the actuator causes the reusable applicator base to re-engage and retain the sharp carrier.

38. The assembly of clause 37, wherein re-engagement of the sharp carrier by the reusable applicator base causes the retraction spring to recompress and reload.

39. A method of using an assembly comprising a reusable applicator and a first sensor control device, the reusable applicator comprising a housing, an actuator configured to move in a distal direction relative to the housing, a sharp carrier releasably coupled with a first sharp module, and a reusable applicator base releasably engaged with a first disposable sensor carrier, the method comprising:
placing the reusable applicator against a skin surface and applying a first force on the actuator to advance the first sensor control device from a first position within the reusable applicator to a second position adjacent to the skin surface;
removing the reusable applicator from the skin surface and leaving behind the first sensor control device on the skin surface; and
applying a second force on the actuator to eject the first sharp module, the first disposable sensor carrier from the reusable applicator.

40. The method of clause 39, wherein the reusable applicator further comprises a drive spring having a first end in contact with a firing pin, the method further comprising causing the drive spring to expand in response to applying the first force on the actuator.

41 The method of clause 40, wherein the reusable applicator further comprises a retraction spring disposed within the sharp carrier.

42. The method of clause 41, wherein the reusable applicator base comprises one or more carrier lock arms configured to engage the sharp carrier.

43. The method of clause 42, further comprising causing the reusable applicator base, the retraction spring, the sharp carrier, the sharp module, the disposable sensor carrier, and the first sensor control device to advance in the distal direction towards the skin surface in response to expansion of the drive spring.

44. The method of clause 43, further comprising:
causing the one or more carrier lock arms to disengage from the sharp carrier as the first sensor control device is advanced from the first position to the second position; and
causing the retraction spring to expand after the one or more carrier lock arms are disengaged from the sharp carrier.

45. The method of any of clauses 39 to 44, wherein the sharp module comprises a sharp hub configured to engage with the sharp carrier.

46 The method of clause 45, wherein the sharp module further comprises a sharp.

47. The method of any of clauses 39 to 46, wherein the first disposable sensor carrier comprises one or more latches configured to couple with one or more corresponding ledges of the reusable applicator base.

48. The method of any of clauses 39 to 47, wherein the actuator is at a first height relative to the housing before applying the first force, wherein the actuator is configured to return to a second height relative to the housing after applying the first force, and wherein the second height is greater than the first height.

49. The method of any of clauses 39 to 48, further comprising:
causing a distal portion of the actuator to contact the sharp carrier in response to applying the second force on the actuator.

50 The method of clause 49, wherein the sharp carrier comprises one or more sharp carrier retention arms, the method further comprising:
causing the one or more sharp carrier retention arms to spread apart and disengage from the sharp module in response to the contact by the distal portion of the actuator with the sharp carrier.

51. The method of any of clauses 39 to 50, further comprising:
loading a second sharp module and a second disposable sensor carrier into the reusable applicator through a distal end of the reusable applicator.

52. The method of clause 51, further comprising:
loading a second sensor control device into the reusable applicator through the distal end of the reusable applicator after loading the second sharp module and the second disposable sensor carrier into the reusable applicator.

53. The method of clause 51, further comprising:
loading a second sensor control device into the reusable applicator through the distal end of the reusable applicator while loading the second sharp module and the second disposable sensor carrier into the reusable applicator.

In summary, an assembly and method for delivery of an analyte sensor including a reusable applicator having a proximal portion and a distal portion are disclosed. The reusable applicator can include a housing, a sensor carrier configured to releasably receive the first analyte sensor, a sharp carrier configured to releasably receive a sharp module, and an actuator movable relative to the housing. The actuator can include three positions: a first position with the sensor carrier and the sharp carrier are at the proximal portion of the reusable applicator, a second position with the sensor carrier and the sharp carrier are at the distal portion of the reusable applicator for delivery of the first analyte sensor, and a third position with the sensor carrier at the distal portion of the reusable applicator and the sharp carrier at the proximal portion of the reusable applicator after delivery of the first analyte sensor from the reusable applicator, wherein the first position, the second position, and the third position are different, and wherein the actuator is configured to be returned from the third position to the first position for delivery of another analyte sensor.

The description encompasses and expressly envisages methods that are non-surgical, non-invasive methods and methods that are implemented outside the body. The methods are typically implemented by a user who is not required to be a medical professional.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the scope of the claims by features, functions, steps, or elements that are not within that scope.

1. An assembly for delivery of an analyte sensor comprising:
a reusable applicator configured to deliver a first analyte sensor, the reusable applicator having a proximal portion and a distal portion and including:
a housing;
a sensor carrier configured to releasably receive the first analyte sensor;
a sharp carrier configured to releasably receive a sharp module, wherein the sharp module is configured to be removed from the proximal portion of the reusable applicator; and
an actuator moveable relative to the housing, the actuator having:
a first position with the sensor carrier and the sharp carrier at the proximal portion of the reusable applicator,
a second position with the sensor carrier and the sharp carrier at the distal portion of the reusable applicator for delivery of the first analyte sensor from the reusable applicator, and
a third position with the sensor carrier at the distal portion of the reusable applicator and the sharp carrier at the proximal portion of the reusable applicator after delivery of the first analyte sensor,
wherein the first position, the second position, and the third position are different, and wherein the actuator is configured to be returned from the third position to the first position for delivery of another analyte sensor.

2. The assembly for delivery of an analyte sensor of claim 1, wherein the reusable applicator further includes a drive spring to move the sensor carrier and the sharp carrier from the proximal portion to the distal portion and a retraction spring to move the actuator to the third position.

3. The assembly for delivery of an analyte sensor of claim 2, wherein the drive spring is actuated by movement of the actuator from the first position to the second position.

4. The assembly for delivery of an analyte sensor of claim 2, wherein the retraction spring is actuated by movement of the sensor carrier from the proximal position to the distal position of the reusable applicator.

5. The assembly for delivery of an analyte sensor of claim 1, wherein the reusable applicator further includes a latch to hold the sensor carrier at the distal portion of the reusable applicator when the actuator is moved from the second position toward the third position.

6. The assembly for delivery of an analyte sensor of claim 1, wherein with the actuator in the third position, the sharp carrier is accessible from the proximal portion of the reusable applicator to release the sharp module.

7. The assembly for delivery of an analyte sensor of claim 1, wherein the actuator further comprises a visual indicator of a position of the actuator.

8. The assembly for delivery of an analyte sensor of claim 7, wherein the actuator includes a button configured to extend a first predetermined length relative the housing in the first position, a second predetermined length relative the housing in the second position, and a third predetermined length relative the housing in the third position, and wherein the third predetermined length is greater than the first predetermined length and the first predetermined length is greater than the second predetermined length.

9-11. (canceled)

12. The assembly for delivery of an analyte sensor of claim 1, further comprising a sealable container to package the reusable applicator assembly.

13. The assembly for delivery of an analyte sensor of claim 12, wherein the sealable container has a low moisture vapor transition rate.

14. The assembly for delivery of an analyte sensor of claim 13, wherein the sealable container is configured to eliminate the need for a desiccant.

15. The assembly for delivery of an analyte sensor of claim 1, further comprising an applicator cap sealingly coupled to the housing with a gasketless seal.

16. A method of using an assembly for delivery of an analyte sensors, comprising:
providing a reusable applicator having a proximal portion and a distal portion, the reusable applicator including a housing, a sensor carrier having a first analyte sensor releasably received therein, a sharp carrier having a sharp module releasably received therein, and an actuator moveable relative to the housing, wherein the sharp module is configured to be removed from the proximal portion of the reusable applicator;
moving the actuator of the reusable applicator assembly from a first position to a second position to move the sensor carrier and the sharp carrier from the proximal portion of the reusable applicator toward the distal portion of the reusable applicator to deliver the analyte sensor from the sensor carrier;
moving the sharp carrier from the distal portion of the reusable applicator toward the proximal portion of the reusable applicator and moving the actuator of the reusable applicator assembly to a third position after delivery of the first analyte sensor; and
returning the actuator from the third position to the first position for receipt of another analyte sensor for delivery;
wherein the first position, the second position, and the third position are different.

17. The method of using an assembly for delivery of an analyte sensor of claim 16, wherein the reusable applicator further includes a drive spring to move the sensor carrier and the sharp carrier from the proximal portion to the distal portion.

18. The method of using an assembly for delivery of an analyte sensor of claim 16, wherein the reusable applicator further includes a retraction spring to move the actuator to the third position.

19. The method of using an assembly for delivery of an analyte sensor of claim 17, wherein after returning the actuator from the third position to the first position the method further includes:
reloading, using the actuator of the reusable applicator assembly, the retraction spring by moving the sharp carrier from the proximal portion of the reusable applicator to the distal portion of the reusable applicator; and
reloading the drive spring by moving the sensor carrier and the sharp carrier from the distal portion of the reusable applicator to the proximal portion of the reusable applicator.

20. The method of using an assembly for delivery of an analyte sensor of claim 16, wherein the reusable applicator further includes a latch to hold the sensor carrier at the distal portion of the reusable applicator when the actuator moves from the second position toward the third position.

21. The method of using an assembly for delivery of an analyte sensor of claim 16, further comprising accessing the sharp carrier from the proximal portion of the reusable applicator for releasing the sharp module.

22. The method of using an assembly for delivery of an analyte sensor of claim 16, wherein the reusable applicator includes a visual indicator of a position of the actuator.

23. The method of using an assembly for delivery of an analyte sensor of claim 16, wherein the actuator includes a button, and the method further comprises opening the button to access and remove the first sharp module when the actuator is in the third position.

24-53. (canceled)
